(19) United States
(12) Patent Application Publication
(10) Pub. No.: US 2020/0299340 A1
(43) Pub. Date: Sep. 24, 2020

(54) FUSION PROTEINS COMPRISING DETECTABLE TAGS, NUCLEIC ACID MOLECULES, AND METHOD OF TRACKING A CELL

(71) Applicant: ICAHN SCHOOL OF MEDICINE AT MOUNT SINAI, New York, NY (US)

(72) Inventors: Brian BROWN, New York, NY (US); Aleksandra WROBLEWSKA, New York, NY (US)

(21) Appl. No.: 16/641,959

(22) PCT Filed: Aug. 24, 2018
(86) PCT No.: PCT/US2018/047996

§ 371 Date: Feb. 25, 2020

Related U.S. Application Data
(60) Provisional application No. 62/550,086, filed on Aug. 25, 2017.

(51) Int. Cl. C07K 14/435; C07K 14/705; C12N 15/10; G01N 33/58

(52) U.S. Cl. CPC C07K 14/43595; C07K 14/70578; C07K 2319/60; G01N 33/58; C07K 2319/035; C12N 15/1044

(57) ABSTRACT

The present invention is directed to a fusion protein comprising a scaffold protein and a series of two or more epitopes, where the distinct epitopes are recognized by distinct antibodies, and where the series of epitopes forms a detectable protein tag. The present invention further relates to a nucleic acid molecule encoding a nucleic acid sequence encoding the fusion protein, as well as vectors comprising the nucleic acid molecule. Methods of tracking a cell and kits using such vectors are also disclosed.

This application claims the priority benefit of U.S. Provisional Patent Application Ser. No. 62/550,086, filed Aug. 25, 2017, which is hereby incorporated by reference in its entirety.

This invention was made with government support under Grant Numbers RO1AI113221 and R33CA182377 awarded by the National Institutes of Health. The United States Government has certain rights in the invention.

### FIELD OF THE INVENTION

The present invention relates to fusion proteins comprising detectable tags, nucleic acid molecules encoding the fusion proteins, and a method of tracking a cell or gene vector.

### BACKGROUND OF THE INVENTION

There is a major need for methods and reagents useful in single-cell tracking of hundreds of cells within a population, which cannot be achieved with any currently available technology.

An important application of cell tracking technology is in genetic screening assays, which aim to identify and select for individual cells that comprise a phenotype of interest in a genetically modified population. Such assays typically utilize knockout (“KO”), knockdown (“KD”), or overexpression (“OE”) vectors encoding a CRISPR guide RNA (“gRNA”), shRNA, or cDNA targeting a specific gene or gene product.

One method to determine whether a specific vector has been introduced into a cell is through the use of a reporter-gene (e.g., Green Fluorescent Protein (“GFP”) and Yellow Fluorescent Protein (“YFP”)), which provides the opportunity to track genetically modified cells using microscopy, flow cytometry, and various other detection means (Tsien, “The Green Fluorescent Protein,” Annu. Rev. Biochem. 67:509-44 (1998)). However, spectral overlap limits the utility of this approach to at most 4 reporter genes (Livet et al., “Transgenic Strategies for Combinatorial Expression of Fluorescent Proteins in the Nervous System,” Nature 450:56-62 (2007)). Moreover, KO/KD/OE of every gene in a genome in distinct experimental or environmental conditions is cumbersome, costly, and time consuming. This has led to an increasing demand for technologies and methodologies that enable pooling of vectors to determine the functions of hundreds of genes simultaneously in a single experimental system (Blakely et al., “Pooled Lentiviral shRNA Screening for Functional Genomics in Mammalian Cells,” Methods Mol. Biol. 781:161-182 (2011)).

Genetic barcoding technology in combination with deep-sequencing enables high-throughput evaluation of a population of cells (Lu et al., “Tracking Single Hematopoietic Stem Cells In Vivo Using High-Throughput Sequencing in Conjunction with Viral Genetic Barcoding,” Nat. Biotechnol. 29:928-934 (2011) and Bystrykh et al., “Counting Stem Cells: Methodological Constraints,” Nat. Methods 9:567-574 (2012)). Unique nucleotide sequences can be incorporated into a vector or, alternatively, when the vector encodes an shRNA or gRNA (in the case of CRISPR (Mali et al., “RNA-Guided Human Genome Engineering via Cas9,” Science 339:823-826 (2013) and Cong et al., “Multiplex Genome Engineering Using CRISPR/Cas Systems,” Science 339:819-23 (2013))), the shRNA or gRNA sequence becomes the barcode (Blakely et al., “Pooled Lentiviral shRNA Screening for Functional Genomics in Mammalian Cells,” Methods Mol. Biol. 781:161-182 (2011); Wang et al., “Genetic Screens in Human Cells Using the CRISPR-Cas9 System,” Science 343:80-84 (2014); Chung et al., “Cbx8 Acts Non-Canonically with Wdr5 to Promote Mammary Tumorigenesis,” Cell Rep. 16:472-486 (2016); Sidik et al., “A Genome-Wide CRISPR Screen in Toxoplasma Identifies Essential Apicomplexan Genes,” Cell 166:1423-1435 (2016); Parnas et al., “A Genome-Wide CRISPR Screen in Primary Immune Cells to Dissect Regulatory Networks,” Cell 162:675-686 (2015); Wang et al., “Identification and Characterization of Essential Genes in the Human Genome,” Science 350:1096-1101 (2015); Sanjana et al., “High-Resolution Interrogation of Functional Elements in the Noncoding Genome,” Science 353:1545-1549 (2016); Zhang et al., “A CRISPR Screen Defines a Signal Peptide Processing Pathway Required by Flaviviruses,” Nature 535:164-168 (2016); and Marceau et al., “Genetic Dissection of Flaviviridae Host Factors Through Genome-Scale CRISPR Screens,” Nature 535:159-163 (2016)). Cells can be transduced with hundreds of vectors simultaneously, and the frequency of cells carrying each vector can be determined by deep-sequencing.

Unfortunately, DNA barcoding has major limitations. One significant limitation being that the read-out is performed on the bulk cell population, which means that single cell phenotypes cannot be determined. This is a problem because KO/KD does not occur in 100% of the cell population. Thus, analyzing in bulk includes a mixture of cells with and without the genetic perturbation. Because DNA barcoding requires DNA to be extracted from the cells to analyze the barcode, the cells must be killed for analysis to be performed. This prevents longitudinal analysis of the cells, or selection of cells carrying a specific barcode. Another major limitation is that DNA barcoding requires selection of the cells based on single phenotypes, predominately cell fitness. More informative phenotypes, such as upregulation or downregulation of key genes, cannot be included in a genetic screen using DNA barcodes. Another major limitation of DNA barcoding is that a fairly penetrant phenotype is needed to detect over background.

Thus, there exists a need for a high-throughput single-cell tracking technology, which would enable multiparameter phenotyping and single-cell longitudinal analysis.

The present invention is directed to overcoming deficiencies in the art.

### SUMMARY OF THE INVENTION

A first aspect of the present invention relates to a fusion protein comprising a scaffold protein and a series of two or more distinct epitopes, where the distinct epitopes are recognized by distinct antibodies, and where the series of epitopes forms a detectable protein tag.

Another aspect of the present invention relates to a nucleic acid molecule comprising (i) a first nucleic acid sequence encoding a fusion protein comprising a scaffold protein and a series of two or more distinct epitopes, where the distinct epitopes are recognized by distinct antibodies, and where the series of epitopes forms a detectable protein tag and (ii) a first promoter operably linked to the first nucleic acid sequence.

A further aspect of the present invention relates to a vector comprising the nucleic acid molecule according to the second aspect of the invention.

Another aspect of the present invention relates to a method of tracking a cell. This method involves providing a plurality of vectors according to the present invention; providing a population of cells; contacting the population of cells with the plurality of vectors under conditions effective for transduction; contacting the transduced cells with labeling molecules capable of binding the two or more epitopes of each fusion protein of each of the plurality of vectors; and detecting the labeling molecules to track the transduced cells.

A further aspect of the invention relates to a kit comprising a library of vectors comprising the nucleic acid molecule of the present invention, where each vector comprises a different series of two or more distinct epitopes.

The present invention provides a novel technology for vector tracking and phenotypically indexing cells. The technology involves the assembly of various epitopes into series of protein barcodes (“Pro-Codes” or “PCs”). Each Pro-Code, when used as a unique molecular identifier (FIGS. 1A-1B), enables simultaneous tracking and phenotypic analysis of cells which have been transduced with thousands of different genetic effector molecules (e.g., cDNA, shRNA, or CRISPR gRNA). The Pro-Code technology of the present application also facilitates high-content annotations of gene functions in a manner not possible with existing technology and has wide-spread applications in experimental biology. The Examples of the present application (infra) demonstrate the use of Pro-Code identifiers to phenotypically distinguish cells transduced with more than one hundred different gene transfer vectors.

### BRIEF DESCRIPTION OF THE DRAWINGS

FIGS. 1A-1U show single cell analysis of Pro-Code expressing populations. FIG. 1A is a schematic of one embodiment of Protein Barcode (Pro-Code) vectors of the present invention. Linear epitopes (n) are assembled in combinations (r) to generate a higher multiple set of Pro-Codes (C). FIG. 1B is a schematic of one embodiment of Pro-Code vector cell transduction, staining, and analysis. In FIGS. 1C, 1E, 1F, and 1I, 293 T-cells were transduced with a library of 19 different Pro-Code vectors. FIG. 1C shows staining of individual epitopes E1-E10. FIG. 1D is a heatmap showing the relative expression of epitopes E1-10 when 293T cells were transduced with 18 different Pro-Code expressing vectors, stained with metal-conjugated antibodies specific for each epitope, and analyzed by CyTOF. FIG. 1E shows the cell yields for each of the 18 unique Pro-Code populations. Data is plotted as a function of the barcode separation threshold. FIG. 1F shows shows individual staining for all 10 epitopes shown for one of the debarcoded Pro-Code populations (E3+E4+E5) in FIG. 1E; positive staining shown in grey (histograms). FIG. 1G shows viSNE clustering of the data described in FIG. 1D. FIG. 1H illustrates individual viSNE plots showing expression of each of the indicated epitopes from the experiment described in FIG. 1D. Expression level is scaled from high to low (yellow to dark purple). In FIG. 1I, 293T cells were transduced at low MOI with a pool of 14 lentiviral vectors each encoding a unique Pro-Code created by assembling 10 epitope tags in combination of 4. Shown are viSNE visualization plots colored by the expression of each unique epitope from low to high. FIGS. 1J-1M show viSNE clustering with expression of each epitope (E1-E10) colored from high to low (red to blue) in 293T cells (FIG. 1J), Jurkat T-cells (FIG. 1K), THP1 monocytes (FIG. 1L), and 4T1 mammary gland carcinoma cells (FIG. 1M) transduced with a pool of 120 different Pro-Code vectors, and analyzed by CyTOF. FIG. 1N is a heatmap showing epitope (“E”) expression for each of the 120 identified Pro-Code cell populations in 293T cells. All data is representative of 3 independent experiments. FIGS. 1O-1R are heatmaps showing the relative expression of each linear epitope in Jurkat (FIG. 1O), THP1 monocytes (FIG. 1P), and 4T1 mammary gland breast cancer cells (FIG. 1Q) transduced with a library of 120 different Pro-Code vectors and analyzed by CyTOF. Heatmaps show the relative expression of each epitope for all Pro-Code cell populations (yellow:high, purple:low) and are representative of 3 independent biological experiments. FIG. 1R shows the frequency distribution of 120 Pro-Codes in 293T cells. Data is shown as percent of a log scale. FIGS. 1S-1U illustrate the resolution of 364 Pro-Code expressing populations. FIG. 1S shows histograms of 293T cells transduced with 364 different Pro-Code expressing vectors, stained with metal-conjugated antibodies specific for each epitope (E1-E14), and analyzed by CyTOF. FIG. 1T shows individual viSNE plots showing expression of each of the indicated epitopes from the experiment described in FIG. 15. Expression level is scaled from high to low (yellow to dark purple). FIG. 1U shows the frequency distribution of 364 Pro-Codes in 293T cells. Data is shown as percent on a log scae.

FIGS. 2A-2D show the analysis of Pro-Code labeled breast tumors. FIG. 1A is a schematic of in vivo tumor studies. Balb/c (WT) or Rag1^{-/-} mice were inoculated in the mammary fat pad with 50,000 4T1 cells transduced with a pool of 120 different Pro-Code vectors. Mice were sacrificed 14 days later and the Pro-Code distribution was analyzed by CyTOF (8 to 10 tumors analyzed per group). FIG. 2B shows the frequency of each Pro-Code expressing population in tumors from wild-type and Rag1^{-/-} mice. Shown is the median±interquartile range (8-10 tumors/mouse group). Also included is the frequency of each Pro-Code in the 4T1 cells prior to inoculation (Pre-inoculation). FIG. 2C shows the distribution of the Pro-Code populations among each tumor. Data is presented in radar plots. The distance from the center represents the frequency of a Pro-Code population (each color represents a tumor, each quadrant corresponds to cells expressing a different Pro-Code). FIG. 2D shows the frequency of the 10 most abundant populations in each individual tumor. On the Y-axis are individual tumors from WT (W) or Rag1^{-/-} (R) mice. Also shown are the 10 most abundant Pro-Codes in the 4T1 cells Pre-inoculation. Numbers in the bars correspond to Pro-Code identifications.

FIGS. 3A-3F show high content phenotypic analysis of monocytic cells engineered with a Pro-Code/CRISPR library. FIG. 3A is a schematic of the Pro-Code/CRISPR phenotypic analysis of THP1 monocytes. 96 lentiviral vectors were generated encoding unique Pro-Code and CRISPR gRNA pairs. Vectors were packaged individually, then pooled, and used to transduce THP1-Cas9 cells. Ten days later, cells were analyzed by CyTOF for expression of the Pro-Code epitopes and the indicated cell surface protein. FIG. 3B shows the expression of the indicated proteins on each Pro-Code/CRISPR cell population. Shown are representative histograms for each Pro-Code population. The Y-axis on histograms represents cell count normalized by protein detection channel. FIG. 3C is a heatmap representation of the relative percent of protein negative cells for each Pro-Code population. All data is representative of 2 independent experiments. FIGS. 3D-3F show the phenotypic analysis of monocytic cells engineered with a Pro-Code/CRISPR library. In FIGS. 3D-3F, 96 lentiviral vectors were generated encoding unique Pro-Code and CRISPR gRNA pairs. Vectors were either packaged individually, then pooled or packaged as a pool with a low homology transfer vector (pCCLsin.PPT.hPGK.GFP) spike. Either library was used to transduce THP1-Cas9 cells. Two weeks later, cells were analyzed by CyTOF expression for the Pro-Code epitopes and the indicated cell surface proteins. FIG. 3D shows the expression of the indicated proteins on each Pro-Code/CRISPR population from cells transduced with the vector library generated from individually packaged vectors. Shown are representative histograms for each Pro-Code population. The Y-axis on histograms represents cell count normalized by protein detection channel. FIG. 3E shows the expression of the indicated proteins on each Pro-Code/CRISPR cell population from cells transduced with a vector library produced as a pool. Shown are representative histograms for each Pro-Code population. The Y-axis on histograms represents cell count normalized by protein detection channel. FIG. 3F shows the percentage of positive (blue) and negative/low (red) cells for each measured protein in the indicated Pro-Code/CRISPR populations.

FIGS. 4A-4L show the analysis of phospho-STAT signaling in Pro-Code/CRISPR engineered cells. FIG. 4A is a schematic overview of phospho-signaling downstream of the IFNg receptor, GM-CSF receptor (CD116), and IL-6 receptor (CD126). FIG. 4B shows representative histograms (n=3 independent experiments) of THP1-Cas9 cells stimulated with IFNg, GM-CSF, IL-6, or PBS (CTRL) stained with metal-conjugated antibodies specific for pSTAT1, pSTAT3, and pSTAT5, and analyzed by CyTOF. FIG. 4C is a schematic of the Pro-Code/CRISPR library used in (FIGS. 4D, 4F, and 4J). FIG. 4D is the viSNE visualization of 24 Pro-Code/CRISPR populations in THP1-Cas9 cells transduced with 24 Pro-Code/CRISPR vectors targeting four cell surface receptor genes. Cells were stimulated with the indicated cytokine and analyzed for the Pro-Codes and pSTAT1 and pSTAT3 by CyTOF. The viSNE visualization is colored by the target gene: green: IFNGR1, blue: IFNGR2, purple: IL6R, orange: GM-CSF receptor, grey: control. FIG. 4E is a viSNE visualization of 24 Pro-Code/CRISPR populations colored by the target: blue:IFNGR1, purple:IFNGR2, green:LILR6, orange:GM-CSF receptor, grey:control of THP1-Cas9 cells transduced with a Pro-Code/CRISPR library as described in FIG. 4D and treated with GM-CSF. Data shown is representative of 3 independent experiments. FIG. 4F shows the expression of pSTAT1 and pSTAT5 in each Pro-Code expressing cell population after stimulation with GM-CSF or IFNg; CTRL refers to cells treated with PBS. Bar plots present the mean intensity (“MI”). Each point is a different Pro-Code/gRNA. FIG. 4G shows the relative expression of pSTAT1 and pSTAT5 levels across all CRISPR/Pro-Code populations after stimulation with GM-CSF or IFNg; CTRL refers to cells treated with PBS. FIG. 4H shows the phosphorylation of STAT1 and STAT3 of THP1-Cas9 cells transduced with a Pro-Code/CRISPR library as described in FIG. 4D and stimulated with IL-6. CTRL refers to cells treated with PBS. Data shown is representative of 3 independent experiments. FIG. 4I shows the expression of pSTAT1 and pSTAT3 in each Pro-Code-expressing cell population after stimulation with IL-6; CTRL refers to cells treated with PBS. Bar plots present the mean intensity (“MI”). FIG. 4J shows the relative expression of pSTAT1 and pSTAT3 levels across all CRISPR/Pro-Code populations after stimulation with IL-6; CTRL refers to cells treated with PBS. FIG. 4K shows levels of pSTAT1 and pSTAT5 after stimulation with IFNγ and GM-CSF, respectively, in different Pro-Code/CRISPR cell populations; representative histograms are shown. Y-axis represents relative cell count. FIG. 4L shows viSNE visualization of pSTAT1 and pSTAT5 levels after stimulation with GM-CSF or IFNγ; CTRL refers to cells treated with PBS. The Pro-Code/CRISPR identity of each cluster can be found in FIG. 4D. Data is representative of 3 independent experiments.

FIGS. 5A-5O illustrate a Pro-Code/CRISPR screen for genes conferring sensitivity or resistance to antigen-dependent T-cell killing. FIG. 5A is a schematic diagram of the immune editing co-culture system and the Pro-Code/CRISPR library used in this study. 4T1 cells (+/−Cas9, +/−GFP/RFP) were transduced with a library of 56 Pro-Code/CRISPR vectors, co-cultured with activated Jedi T-cells, and analyzed by CyTOF. FIG. 5B are representative dotplots showing the frequency of GFP⁺ and RFP⁺ 4 T1 cells measured by flow cytometry. Jedi 1:2-2-fold multiple of T cells to cancer cells, Jedi 1:10-10-fold multiple of T-cells to cancer cells. FIG. 5C are representative dotplots showing the frequency of GFP+ and RFP+ 4T1-Cas9 cells measured by flow cytometry. FIG. 5D shows the viSNE visualization of the 4T1-GFP and 4T1-RFP Pro-Code populations co-cultured alone or with activated Jedi T cells. Each cluster corresponds to a different Pro-Code. FIG. 5E shows the viSNE visualization of the 4T1-GFP-Cas9 and 4T1-RFP-Cas9 Pro-Code populations co-cultured alone or with activated Jedi T cells. Each cluster corresponds to a different Pro-Code. FIG. 5F shows the viSNE visualization of 56 Pro-Code/CRISPR populations (GFP-4T1-Cas9, Jedi 1:10) colored by the target: orange=B2m, cyan=Ifngr2, purple=scramble, navy=others. FIGS. 5G-5H show the frequency of each Pro-Code/CRISPR populations among the GFP-4T1-Cas9 (FIG. 5G) and RFP-4T1-Cas9 (FIG. 5H) cells in the absence (no Jedi) or presence (Jedi 1:2, Jedi 1:10) of GFP-specific Jedi T-cells. In FIG. 5I, GFP- or RFP-4T1-Cas9 cells were transduced with gRNAs targeting B2m or Ifngr2, and co-cultured with different ratios of activated Jedi T-cells. The frequency of GFP+ and RFP+ cells was measured by flow cytometry. FIG. 5I shows representative dotplots from three different experiments. FIG. 5J shows the analysis of H2Kd expression on the 4T1-GFP (green) and 4T1-RFP (red) cells from FIG. 5I Expression of H2Kd on Jedi T-cells is shown as a reference (grey). FIG. 5K shows GFP and H2Kd (MHC class I) expression on 4T1-Cas9-GFP cells expressing gRNAs targeting B2m, Ifngr2 and all other genes. FIG. 5L shows GFP and H2Kd expression levels Pro-Code/CRISPR populations in GFP-4T1-Cas9 cells resisting T-cell killing (Jedi 1:10). FIG. 5M shows NGFR and H2Kd (MHC class I) expression on 4T1-Cas9-RFP cells expressing gRNAs targeting B2m, Ifngr2, and other genes. FIG. 5N shows GFP and H2Kd expression on selected Pro-Code cell populations (from FIG. 5L). Data in FIG. 5 is representative of 3 independent experiments. In FIG. 5O, 4T1-Cas9-GFP, and 4T1-Cas9-mCherry cells expressing scramble gRNA were co-cultured with activated Jedi T-cells (Jedi 1:5). On day 3, extent of killing of GFP cells as well as expression of H2Kd was assessed by flow cytometry. Plots are representative of 5 independent experiments.

FIGS. 6A-6M show Pro-Code/CRISPR analysis of select IFNγ-inducible genes in cancer cell killing by antigen-specific T-cells. In FIGS. 6A-6F, 4T1-Cas9-GFP and 4T1-Cas9-mCherry cells were transduced with a library of 56 Pro-Code/CRISPR vectors, mixed in a 1:1 ratio, and co-cultured with activated Jedi T-cells. On day 3, cells were collected, stained with metal-conjugated antibodies for the Pro-Code epitopes, as well as GFP, mCherry, CD45 and MEW class I (H2Kd), and PD-L1, and analyzed by CyTOF. FIG. 6A shows representative dotplots showing the frequency of 4T1-Cas9-GFP and 4T1-Cas9-mCherry cells measured by CyTOF; no Jedi − no T-cells added, + Jedi − 4-fold excess of T cells over cancer cells. FIGS. 6B-6C are histograms showing PDL1 (FIG. 6B) and H2Kd (FIG. 6C) expression in the bulk GFP⁺ and mCherry⁺ cell populations. FIGS. 6D-6E show viSNE visualizations and histograms showing PDL1 (FIG. 6D) and H2Kd (FIG. 6E) expression of individual Pro-Code/CRISPR populations among the mCherry⁺ cells. FIG. 6F shows the fold enrichment of Psmb8, Rtp4, and scramble Pro-Code/CRISPR populations (+ Jedi vs. no Jedi conditions) shown as a function of % killing by Jedi T-cells. Each dot is from an independent experiment with two different ratios of Jedi to cancer cells. Four independent experiments were performed. FIG. 6G is a graph of GFP-4T1-Cas9 cells transduced with gRNAs targeting Psmb8, Rtp4, or scramble gRNA. The frequency of GFP⁺ cells in the absence (no Jedi) or presence (Jedi 1:1, Jedi 1:2, Jedi 1:5) of Jedi T cells was determined by flow cytometry. Bar graphs present the mean±standard deviation (n=3). 4T1-Cas9-mCherry cells were used as control. Note that the percent of surviving cells is dependent on CRISPR knockout efficiency, and is thus not quantitative, as indicated by FIG. 6J. FIG. 6H shows representative dotplots of 4T1-Cas9-GFP and 4T1-Cas9-mCherry cells transduced with lentiviral encoding gRNAs targeting Psmb8, Rtp4, or scramble sequences. Cells were mixed in a 1:1 ratio and co-cultured with activated Jedi T-cells. The frequency of GFP⁺ and mCherry⁺ cells was determined by flow cytometry. Data is representative of three independent experiments and corresponds to the bar graph shown in FIG. 6G. FIG. 6I is a schematic overview of the Psmb8 and Rtp4 validation approach. FIG. 6J shows dotplots of 4T1-Cas9-GFP cells transduced with a vector encoding a Psmb8, Rtp4, or scramble gRNA selected as shown in FIG. 6I and mixed with activated Jedi T-cells, and cultured for 3 days. Frequency of GFP⁺ and mCherry⁺ cells in the absence (no Jedi) or presence (+Jedi) of Jedi T-cells is shown. Dotplots are representative of 2 independent experiments. FIG. 6K is a Western blot for Psmb8 and β-actin. Cells were generated as described in FIG. 6I. The cells were either left untreated with 10 ng/ml IFNγ, and 2 days later protein was extracted for western blot. FIG. 6L shows sequence analysis of the Rtp4 genome locus targeted by the Rtp4 gRNA from cells selected as described in FIG. 6I. DNA was extracted from the cells, the locus was PCR amplified, and the PCR product was cloned into TOPO cloning vector, and transformed into TOP10 bacteria. Colonies were randomly selected, plasmid DNA was minipreped and Sanger sequenced. The parental target sequence (SEQ ID NO: 1) is identified. Sequencing analysis of 19 clones is also shown (SEQ ID NOs: 2-20). FIG. 6M is a graph showing the measurement of Rtp4 RNA expression. RNA was subject to RT-qPCR using primers specific for Rtp4 and actin (as a control). The graph presents the mean±standard deviation of the AΔCT (n=4). Beta actin was used to normalize, and untreated scramble was used to calibrate.

FIGS. 7A-7B show that GFP can function as a Pro-Code scaffold. In FIG. 7A, three different linear epitopes (Stll, V5, and HA) were fused to the C-terminus of GFP. In FIG. 7B, 293T cells were transduced with the vector in FIG. 7A. Intracellular staining was performed with metal-conjugated antibodies specific for GFP, and the epitopes HA, Stll, and V5. The cells were analyzed by CyTOF.

### DETAILED DESCRIPTION OF THE INVENTION

The present invention is directed to protein barcode (“Pro-Code”) technology. One aspect of the present invention relates to a fusion protein comprising (i) a scaffold protein and (ii) a series of two or more distinct epitopes, where the distinct epitopes are recognized by distinct antibodies, and where the series of epitopes forms a detectable protein tag.

As used herein, the term “scaffold protein” refers to a protein to which amino acid sequences (i.e., the series of two or more distinct epitopes) can be fused. In one embodiment, the two or more distinct epitopes are heterologous to the scaffold protein. In another embodiment, at least one of the two or more epitopes is heterologous to the scaffold protein.

In one embodiment, the scaffold protein is such that it allows the two or more distinct epitopes to be displayed in the fusion protein in a way that the two or more epitopes are accessible to other molecules. In other words, the scaffold protein takes on a conformation that serves as a scaffold for the two or more distinct epitopes to be accessible to other molecules. For example, and without limitation, the scaffold protein is such that it allows the two or more distinct epitopes to be displayed in the fusion protein such that they are accessible to epitope-specific antibodies. In this manner, the two or more distinct epitopes form a detectable protein tag, as discussed in more detail infra.

In one embodiment, the scaffold protein is a reporter protein. As used herein, the term “reporter protein” refers to a protein that is heterologous to a target cell and whose presence indicates successful gene transfer from a vector to the target cell. Reporter proteins are well known in the art and include, for example and without limitation, mutated Nerve Growth Factor Receptor (“dNGFR”) and GFP.

In one embodiment, the scaffold protein is a cell surface protein. The cell surface protein may be a mutated protein, such as a truncated protein. Suitable cell surface proteins include, but are not limited to, Nerve Growth Factor Receptor (“NGFR”) and mutated Nerve Growth Factor Receptor (“dNGFR”). Additional suitable cell surface proteins include, without limitation, CherryPicker™ (Clontech laboratories, Inc.), truncated epidermal growth factor receptor (“EGFR”), CD34, CD19, CD20, CD4, CD45, HA, and CD90 (see, e.g., Wang et al., “A Transgene-Encoded Cell Surface Polypeptide for Selection, in vivo Tracking, and Ablation of Engineered Cells,” Blood 118(5):1255-1263 (2011), which is hereby incorporated by reference in its entirety.

In another embodiment, the scaffold protein is an intracellular protein. In accordance with this embodiment, the scaffold protein is selected from GFP, blue fluorescent protein (“BFP”), yellow fluorescent protein (“EYFP”), and derivatives thereof. Other suitable intracellular proteins include, without limitation, UV Proteins (Sirius, Sandercyanin, shBFP-N158S/L173I), Blue Proteins (Azurite, EBFP2, mKalama1, BFP, mTagBFP2, TagBFP, shBFP), Cyan Proteins (CFP, ECFP, Cerulean, mCerulean3, SCFP3A, CyPet, mTurquoise, mTurquoise2, TagCFP, TFP, mTFP1, monomeric Midoriishi-Cyan, Aquamarine), Green Proteins (GFP, TurboGFP, TagGFP2, mUKG, Superfolder GFP, Emerald, EGFP, monomeric Azami Green, mWasabi, Clover, mNeonGreen, NowGFP, mClover3), Yellow Proteins (YFP, TagYFP, EYFP, Topaz, Venus, SYFP2, Citrine, Ypet, laRFP-ΔS83, mPapaya1, mCyRFP1), Orange Proteins (monomeric Kusabira-Orange, mOrange, mOrange2, mKO1, mKO2), Red Proteins (TagRFP, TagRFP-T, mRuby, mRuby2, mTangerine, mApple, mStrawberry, FusionRed, mCherry, mNectarine, mRuby3, mScarlet, mScarlet-I), Far Red Proteins (mKate2, HcRed-Tandem, mPlum, mRasberry, mNeptune, NirFP, TagRFP657, TagRFP675, mCardinal, mStable, mMaroon1, mGarnet2), Near IR Proteins (iFP1.4, iRFP713 (iRFP), iRFP670, iRFP682, iRFP702, iRFP720, iFP2.0, TDsmURFP, miRFP670), Sapphire-type Proteins (Sapphire, T-Sapphire, mAmertrine), Long Stokes Shift Proteins (mKeima, mBeRFP, LSS-mKate2, LSS-mKate1, LSSmOrange, CyOFP1, Sandercyanin), as well as Photoactivatible Proteins (PA-GFP, PATagRFP, PAmCherryl, PamKate), Photoconvertible Proteins (PS-CFP2, mClavGR2, mMaple, Dendra2, pcDronpa2, mKikGR, mEos2, KikGR1, Meos3.2, Kaede, PsmOrange2, PSmOrange), and Photoswitchable Proteins (rsEGFP2, mIrisFP, rsEGFP, mGeos-M, Dronpa, Dreiklang).

The fusion protein of the present invention includes, in addition to a scaffold protein, a series of two or more distinct epitopes. As used herein, the term “epitope” refers to the portion of an antigenic molecule (e.g., a peptide) that is specifically bound by the antigen binding domain of an antibody or antibody fragment. Epitopes may be linear or conformational. Linear epitopes are formed from contiguous residues and are typically retained upon exposure to a denaturing solvent, whereas conformational epitopes are formed by tertiary folding and are typically lost upon treatment with a denaturing solvent.

In one embodiment, the fusion protein has two distinct epitopes. In another embodiment, the fusion protein has three distinct epitopes. In yet another embodiment, the fusion protein may have more than three distinct epitopes, including 4, 5, 6, 7, 8, 9, or more distinct epitopes. The number of distinct epitopes contained in the fusion protein increases the number of different detectable protein tags available for methods described herein. In one embodiment, the fusion protein has only linear epitopes or only conformational epitopes. In another embodiment, the fusion protein has a combination of both linear and conformational epitopes.

As used herein, an epitope may comprise up to 200 amino acid residues. In one embodiment, the epitope comprises 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 amino acid residues, but typically will not have more than about 42 amino acid residues. In one embodiment, each of the two or more epitopes comprises no more than 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, or 6 amino acid residues.

In another embodiment, each of the two or more epitopes comprises no more than 14 amino, acid residues. In yet another embodiment, each of the two or more epitopes may comprise at least 6, 7, 8, 9, 10, 11, 12, 13, or 14 amino acid residues. In one embodiment, each of the two or more epitopes comprises 6 amino acid residues. In another embodiment, the epitopes may comprise at least 6 amino acid residues, between 6 and 14 amino acid residues, between 6 and 13 amino acid residues, between 6 and 12 amino acid residues, between 6 and 11 amino acid residues, between 6 and 10 amino acid residues, or between 6 and 9 amino acid residues.

Table 1 below provides a list of various suitable epitopes.

TABLE 1
Epitopes

| | | | SEQ |
|---|---|---|---|
| | Amino Acid | Amino Acid | ID |
| Name | Sequence | Quantity | NO: |
| HA | YPYDVPDYA | 9 | 21 |
| FLAG | DYKDDDDK | 8 | 22 |
| VSVg | YTDIEMNRLGK | 11 | 23 |
| V5 | GKPIPNPLLGLDST | 14 | 24 |
| AU1 | DTYRYI | 6 | 25 |
| AU5 | TDFYLK | 6 | 26 |
| S1 | NANNPDWDF | 9 | 27 |
| (Strep I) | | | |
| E | GAPVPYPDPLEPR | 13 | 28 |
| E2 | GVSSTSSDFRDR | 12 | 29 |
| NWS | NWSHPQFEK | 9 | 30 |
| (Strep II) | | | |

In one embodiment, each of the two or more epitopes are selected from HA, FLAG, VSVg, V5, AU1, AU5, Strep I, E, E2, and Strep II.

There are many other known epitopes that would be useful in the fusion protein of the present invention. Other suitable epitopes include, without limitation, those identified in Table 2 below.

TABLE 2
Additional Suitable Epitopes

| | | Amino | SEQ |
|---|---|---|---|
| | | Acid | ID |
| Name | Amino Acid Sequence | Quantity | NO: |
| His | HHHHHH | 6 | 31 |
| c-myc | EQKLISEEDL | 10 | 32 |
| protein | EDQVDPRLIDGK | 12 | 33 |
| C tag | | | |
| Avi | GLNDIFEAQKIEWHE | 15 | 34 |
| B-Tag | QYPALT | 6 | 35 |
| CBP-tag | KRRWKKNFIAVSAANRFKKISSSGAL | 26 | 36 |
| DDDDK-tag | XXXDDDDK* | 8 | 37 |
| Glu-Glu- | EYMPME | 6 | 38 |
| tag | | | |
| HAT | KDHLIHNVHKEFHAHAHNK | 19 | 39 |
| HSV | QPELAPEDPED | 11 | 40 |
| KT3 | KPPTPPPEPET | 11 | 41 |
| Nano-tag | MDVEAWLGARVPLVET | 16 | 42 |
| OLLAS | SGFANELGPRLMGKC | 15 | 43 |
| Rho-tag | MNGTEGPNFYVPFSNKTGVV | 20 | 44 |
| SRT | TFIGAIATDT | 10 | 45 |
| S-tag | KETAAAKFERQHMDS | 15 | 46 |
| T7-tag | MASMTGGQQMG | 11 | 47 |
| Tag-100- | EETARFQPGYRS | 12 | 48 |
| tag | | | |
| TAP-tag | CSSGALDYDIPTTASENLYFQ | 21 | 49 |
| Ty1-tag | EVHTNQDPLD | 10 | 50 |
| Universal | HTTPHH | 6 | 51 |
| Tag | | | |

*where X may be any amino acid

In the fusion protein of the present invention, epitopes are arranged in a series, meaning two or more epitopes coming one right after another in the amino acid sequence forming the fusion protein. In one embodiment, the epitopes are immediately adjacent to each other. In another embodiment, there is a relatively short amino acid spacer sequence between each of the two or more epitopes. This amino acid spacer sequence may comprise 1, 2, 3, 4, 5, 6, 7, 8, 9, 10, 11, 12, 13, 14, 15, 16, 17, 18, 19, 20, or so amino acid sequences. Suitable spacers are well known in the art and are described in more detail at, e.g., Chen et al., “Fusion Protein Linkers: Property, Design and Functionality,” Adv. Drug Deliv. Rev. 65(10):1357-1369 (2013) and Chichili et al., “Linkers in the Structural Biology of Protein-Protein Interactions,” Protein Sci. 22(2):153-167 (2013), which are hereby incorporated by reference in their entirety).

In one embodiment, the amino acid spacer sequence comprises one or more of the following amino acid residues: alanine, glycine, glutamine, serine, threonine, and proline. In one embodiment, the amino acid spacer sequence is a polyglutamine spacer. Suitable spacer sequences include, without limitation, polyglycine, glycine-rich, and glycine-serine (“GS”) linkers. In one embodiment, the spacer sequence is selected from GGGGGG (SEQ ID NO:52), GGGGGGGG (SEQ ID NO:53), GSGSGS (SEQ ID NO:54), and GGGGS (SEQ ID NO:55).

The spacer sequence may comprise multiple copies of any one or more of SEQ ID NOs:52-55. For example, the spacer sequence may comprise (GGGGS)ₙ, where n=2, 3, 4, 5, 6, 7, 8, 9, or 10. In accordance with this embodiment, the spacer sequence is a flexible linker.

In the fusion protein of the present invention, amino acid spacers as discussed supra may also be included to separate the combination of two or more epitopes from the scaffold protein.

In one embodiment, the two or more epitopes are located in the fusion protein downstream of the scaffold protein. In another embodiment, the two or more epitopes are located in the fusion protein upstream of the scaffold protein.

In the fusion protein of the present invention, the two or more epitopes are distinct, meaning distinct from each other. In other words, each epitope is specifically recognized by a different antibody, with one antibody being specific to one epitope in the series and a different antibody being specific to another of the epitopes in the series. The particular combination of epitopes forms a unique detectable protein tag, identifiably distinct from other combinations of epitopes.

As used herein, a “detectable protein tag” refers to a polypeptide tag that may be recognized using any conventional biotechnology techniques known in the art including, but not limited to, standard immunological techniques. For example, a detectable protein tag may be recognized by an antibody.

Another aspect of the present invention relates to a nucleic acid molecule comprising (i) a first nucleic acid sequence encoding a fusion protein comprising a scaffold protein and a series of two or more distinct epitopes, where the distinct epitopes are recognized by distinct antibodies, and where the series of epitopes forms a detectable protein tag and (ii) a first promoter operably linked to the first nucleic acid sequence.

As used herein, the term “operably linked” refers to a nucleic acid sequence placed in a functional relationship with another nucleic acid sequence. For example, a nucleic acid promoter sequence may be operably linked to a nucleic acid sequence encoding a protein or polypeptide if it affects the transcription of the nucleic acid sequence encoding the protein or polypeptide.

The nucleic acid molecule of the present invention comprises a first nucleic acid sequence encoding a fusion protein as described supra.

In addition, the nucleic acid molecule may also further encode a signal peptide. As used herein, the term “signal peptide” or “signal sequence” refers to an amino acid sequence that facilitates the passage of a secreted protein molecule or a membrane protein molecule across the endoplasmic reticulum. In eukaryotic cells, signal peptides share the characteristics of (i) an N-terminal location on the protein; (ii) a length of about 16 to about 35 amino acid residues; (iii) a net positively charged region within the first 2 to 10 residues; (iv) a central core region of at least 9 neutral or hydrophobic residues capable of forming an alpha-helix; (v) a turn-inducing amino acid residue next to the hydrophobic core; and (vi) a specific cleavage site for a signal peptidase (see U.S. Pat. No. 6,403,769, which is hereby incorporated by reference in its entirety).

In one embodiment, the signal peptide comprises 15-30 amino acid residues. Suitable signal peptides are well known in the art and include, without limitation, those identified in Table 3 below.

TABLE 3
Signal Peptides

| | | Amino | SEQ |
|---|---|---|---|
| | Amino Acid | Acid | ID |
| Protein | Sequence | Quantity | NO: |
| NGFR | MGAGATGRAMDGPR | 28 | 56 |
| | LLLLLLLGVSLGGA | | |
| Preproalbumin | MKWVTFLLLL | 19 | 57 |
| | FISGSAFSR | | |
| Pre-IgG light | MDMRAPAQIFGF | 23 | 58 |
| chain | LLLLFPGTRCD | | |
| Prelysozyme | MRSLLILVLC | 19 | 59 |
| | FLPLAALGK | | |
| SPtPA* | MDAMKRGLCCVL | 23 | 60 |
| | LLCGAVFVSPS | | |

*human tissue-type plasminogen activator (amino acids 1-23, accession no. P00750.1)

In one embodiment, the nucleic acid molecule encodes the signal peptide of SEQ ID NO:56 (supra) and the cell surface scaffold protein mutant Nerve Growth Factor Receptor (“dNGFR”).

In one embodiment of the nucleic acid sequence of the present invention, the first promoter operably linked to the first nucleic acid sequence is an inducible promoter. In one embodiment, the first promoter is an RNA polymerase II promoter. Suitable RNA polymerase II promoters include, but are not limited to, EF1a, PGK1, CMV, SFFV, CAG (chimeric Actin/CMV promoter), Ubiquitin C (“Ubc”), SV40, UAS, and Tetracycline response element (“TRE”).

In another embodiment of the nucleic acid sequence of the present invention, the first promoter operably linked to the first nucleic acid sequence is a constitutive promoter.

In one embodiment, the nucleic acid molecule further comprises a second nucleic acid sequence encoding an effector molecule and a second promoter operatively linked to the second nucleic acid sequence.

In one embodiment, the effector molecule is a non-coding regulatory nucleic acid sequence. Suitable non-coding regulatory nucleic acid sequences include, but are not limited to, CRISPR guide RNA and shRNA.

As used herein, the term “guide RNA” refers to an RNA molecule that can bind to a Cas protein and aid in targeting the Cas protein to a specific location within a target polynucleotide (e.g., a DNA). Methods of designing guide RNA (“gRNA”) sequences are well known in the art and are described in more detail in, e.g., U.S. Pat. Nos. 8,697,359 and 9,023,649, both of which are hereby incorporated by reference in their entirety.

When the effector molecule is a non-coding regulatory nucleic acid sequence, the second promoter is an RNA polymerase III promoter. In one particular embodiment, the RNA polymerase III promoter is selected from U6 or H1.

The non-coding regulatory nucleic acid sequence may be a gene-silencing, gene knockdown, or gene knockout nucleic acid sequence.

In one embodiment, the effector molecule is a protein-coding nucleic acid sequence. Suitable protein-coding nucleic acid sequences include cDNA. The cDNA may encode a protein of interest. As used herein, the term “protein of interest” refers to a protein or a polypeptide that is distinct from the fusion protein of the present invention. The protein of interest may be homologous or heterologous to the host cell. The protein of interest may be a wildtype protein, a mutated protein, or a recombinant protein.

In one embodiment, the protein of interest is selected from a hormone, cytokine, chemokine, growth factor, signaling peptide, receptor (e.g., T-cell receptor), antibody, enzyme, transcription factor, epigenetic regulator, metabolic protein, clotting factor, tumor suppressor gene, oncogene, and any other transmembrane/surface protein.

In one embodiment, when the effector molecule is a protein-coding nucleic acid sequence, the second promoter is an RNA polymerase II promoter. Suitable RNA polymerase II promoters are described supra and include, e.g., EF1a, PGK1, CAG, CMV, Ubc, and SFFV.

A further aspect of the present invention relates to a vector comprising the nucleic acid molecule of the present invention.

Translating RNA molecules of the present invention may include the use of cell-based (i.e., in vivo) and cell-free (i.e., in vitro) expression systems. Translation or expression of a fusion protein can be carried out by introducing a nucleic acid molecule encoding a fusion protein into an expression system of choice using conventional recombinant technology. Generally, this involves inserting the nucleic acid molecule into an expression system to which the molecule is heterologous (i.e., not normally present). The introduction of a particular foreign or native gene into a mammalian host is facilitated by first introducing the gene sequence into a suitable nucleic acid vector.

“Vector” is used herein to mean any genetic element, such as a plasmid, phage, transposon, cosmid, chromosome, virus, virion, etc., which is capable of replication when associated with the proper control elements, and/or which is capable of transferring gene sequences into cells. Thus, the term includes cloning and expression vectors, as well as viral vectors. The heterologous nucleic acid molecule is inserted into the expression system or vector in proper sense (5′→3′) orientation and correct reading frame. The vector contains the necessary elements for the transcription and translation of the inserted protein coding sequences.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

A variety of host-vector systems may be utilized to express a (fusion) protein encoding sequence in a cell. Primarily, the vector system must be compatible with the host cell used. Host-vector systems include, but are not limited to, the following: microorganisms such as yeast containing yeast expression vectors; mammalian cell systems infected with virus (e.g., vaccinia virus, adenovirus, lentivirus, retrovirus, adeno-associated virus, transposon, plasmid, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (“mRNA”) translation).

Transcription of DNA is dependent upon the presence of a promoter, which is a DNA sequence that directs the binding of RNA polymerase and thereby promotes mRNA synthesis. Promoters vary in their “strength” (i.e., their ability to promote transcription). For the purposes of expressing a cloned gene it is desirable to use strong promoters to obtain a high level of transcription and, hence, expression of the gene. Depending upon the host cell system utilized, any one of a number of suitable promoters may be used.

Depending on the vector system and host utilized, any number of suitable transcription and/or translation elements, including constitutive, inducible, and repressible promoters, as well as minimal 5′ promoter elements may be used.

The protein-encoding nucleic acid, a promoter molecule of choice, a suitable 3′ regulatory region, and if desired, polyadenylation signals and/or a reporter gene, are incorporated into a vector-expression system of choice to prepare a nucleic acid construct using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety.

The nucleic acid molecule encoding a protein is inserted into a vector in the sense (i.e., 5′→3′) direction, such that the open reading frame is properly oriented for the expression of the encoded protein under the control of a promoter of choice. Single or multiple nucleic acids may be ligated into an appropriate vector in this way, under the control of a suitable promoter, to prepare a nucleic acid construct.

Once the isolated nucleic acid molecule encoding the protein has been inserted into an expression vector, it is ready to be incorporated into a host cell. Recombinant molecules can be introduced into cells via transformation, particularly transduction, conjugation, lipofection, protoplast fusion, mobilization, particle bombardment, or electroporation. The DNA sequences are incorporated into the host cell using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable hosts include, but are not limited to, yeast, fungi, mammalian cells, insect cells, plant cells, and the like.

Typically, an antibiotic or other compound useful for selective growth of the transformed cells only is added as a supplement to the media. The compound to be used will be dictated by the selectable marker element present in the plasmid with which the host cell was transformed. Suitable genes are those which confer resistance to gentamycin, G418, hygromycin, puromycin, streptomycin, spectinomycin, tetracycline, chloramphenicol, and the like. Similarly, “reporter genes” which encode enzymes providing for production of an identifiable compound, or other markers which indicate relevant information regarding the outcome of gene delivery, are suitable. For example, various luminescent or phosphorescent reporter genes are also appropriate, such that the presence of the heterologous gene may be ascertained visually.

In some embodiments, translating the RNA molecule is carried out in a cell-free system. Cell-free expression allows for fast synthesis of recombinant proteins and enables protein labeling with modified amino acids, as well as expression of proteins that undergo rapid proteolytic degradation by intracellular proteases. As described above, exemplary cell-free systems comprise cell-free compositions, including cell lysates and extracts. Whole cell extracts may comprise all the macromolecule components needed for translation and post-translational modifications of eukaryotic proteins. As described above, these components include, but are not limited to, regulatory protein factors, ribosomes, and tRNA.

In one embodiment, the vector is a viral vector. Suitable viral vectors are well known in the art and include, but are not limited to, retrovirus, adenovirus, adeno-associated virus, herpesvirus, influenza virus, and poxvirus vectors.

In one embodiment, the vector is a retrovirus vector. According to one specific embodiment, the retrovirus vector is a lentiviral vector. Lentiviral vectors are well known in the art and are described in more detail in, e.g., U.S. Pat. No. 8,828,727, which is hereby incorporated by reference in its entirety. Other suitable lentiviral vectors include, but are not limited to, HIV-based lentiviral vectors, e.g., an HIV-1 lentiviral vector (see Connolly, “Lentiviruses in Gene Therapy Clinical Research,” Gene Therapy 9(24):1730-1734 (2002), which is hereby incorporated by reference in its entirety), as well as equine infectious anemia virus (EIAV), foamy virus, and simian immunodeficiency virus (SIV). In one embodiment, the lentiviral vector is replication competent. In another embodiment, the lentiviral vector is replication incompetent.

In one embodiment, the vector of the present invention is a knockdown vector. As used herein, the term “knockdown” refers to a process by which the expression of a gene product has been reduced in a host cell. In accordance with this embodiment, the second nucleic acid sequence encodes a gene silencing nucleic acid sequence where the gene silencing nucleic acid sequence is selected from shRNA and cDNA.

As used herein, the term “short hairpin RNA” or “shRNA” refers to an RNA molecule that leads to the degradation of mRNAs in a sequence-specific manner dependent upon complementary binding of the target mRNA. shRNA-mediated gene silencing is well known in the art (see, e.g., Moore et al., “Short Hairpin RNA (shRNA): Design, Delivery, and Assessment of Gene Knockdown,” Methods Mol. Biol. 629:141-158 (2010), which is hereby incorporated by reference in its entirety). shRNA is cleaved by cellular machinery into siRNA and gene expression is silenced via the cellular RNA interference pathway.

As used herein, the term “small interfering RNA” or “siRNA” refers to double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule. siRNA molecules are typically designed to target a region of the mRNA target approximately 50-100 nucleotides downstream from the start codon. Upon introduction into a cell, the siRNA complex triggers the endogenous RNA interference (RNAi) pathway, resulting in the cleavage and degradation of the target mRNA molecule.

As used herein, the term “complementary DNA” or “cDNA” refers to a DNA molecule that has a complementary base sequence to a molecule of a messenger RNA.

In another embodiment, the vector of the present invention is a knockout vector. As used herein, the term “knockout” refers to a process by which the expression of a gene product has been eliminated in a host cell. In accordance with this embodiment, the second nucleic acid sequence encodes a gene silencing nucleic acid sequence where the gene silencing nucleic acid sequence is a CRISPR guide RNA (Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339(6121):819-23 (2013); and Gaj et al., “ZFN, TALEN, and CRISPR/Cas-based Methods for Genome Engineering,” Cell 31(7):397-405 (2013), which are hereby incorporated by reference in their entirety). The use of CRISPR guide RNA in conjunction with CRISPR-Cas9 technology to target RNA has been described in the art (Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339(6121):819-23 (2013); and Gaj et al., “ZFN, TALEN, and CRISPR/Cas-based Methods for Genome Engineering,” Cell 31(7):397-405 (2013), which are hereby incorporated by reference in their entirety).

In yet another embodiment, the vector is an overexpression vector. As used herein, the term “overexpression” refers to a process by which the expression of a gene transcript or gene product has been introduced or enhanced in a host cell. Overexpression of a gene encoding a protein may be achieved by various methods known in the art, e.g., by increasing the number of copies of the gene that encodes the protein, or by increasing the binding strength of the promoter region or the ribosome binding site in such a way as to increase the transcription or the translation of the gene that encodes the protein. In accordance with this embodiment, the second nucleic acid sequence encodes a protein of interest.

Another aspect of the present invention relates to a method of tracking a cell. This method involves providing a plurality of vectors according to the present invention; providing a population of cells; contacting the population of cells with the plurality of vectors under conditions effective for transduction; contacting the transduced cells with labeling molecules capable of binding the two or more epitopes of each fusion protein of each of the plurality of vectors; and detecting the labeling molecules to track the transduced cells.

In the method of the present invention, the population of cells may be a population of mammalian cells, for example, human cells.

In one embodiment, the population of cells may be a population of primary cells. As used herein, the term “primary cells” refers to cells which have been isolated directly from human or animal tissue. Once isolated, they are placed in an artificial environment in plastic or glass containers supported with specialized medium containing essential nutrients and growth factors to support cell survival and/or proliferation. Primary cells may be adherent or suspension cells. Adherent cells require attachment for growth and are said to be anchorage-dependent cells. The adherent cells are usually derived from tissues of organs. Suspension cells do not require attachment for growth and are said to be anchorage-independent cells.

In one embodiment, the population of cells is a population of cell line cells. As used herein, the term “cell line cells” refers to cells that have been continuously passaged over a long period of time and have acquired relatively homogenous genotypic and phenotypic characteristics. Cell lines can be finite or continuous. An immortalized or continuous cell line has acquired the ability to proliferate indefinitely, either through genetic mutations or artificial modifications. A finite cell line has been sub-cultured for 20-80 passages after which the cells have senesced.

In one embodiment, the cells are tumor cells or tumor cell line cells.

In one embodiment, the cells are modified to express a heterologous protein. In accordance with this embodiment, the cells are modified to stably express a Cas9 protein. Suitable modified cell lines include, e.g., THP1-Cas9 cells, Jurkat-Cas9 cells, and 4T1-Cas 9 cells.

In one embodiment, contacting the transduced cells is carried out using in situ hybridization. As used herein, the term “in situ hybridization” or “ISH” refers to a type of hybridization that uses a directly or indirectly labeled complementary DNA or RNA strand, such as a probe, to bind to a specific nucleic acid, such as DNA or RNA, in a sample. When contacting the transduced cells is carried out using in situ hybridization, the labeling molecules may be selected from double stranded DNA (“dsDNA”), single stranded DNA (“ssDNA”), single stranded complementary RNA (“sscRNA”), messenger RNA (“mRNA”), micro RNA (“miRNA”), and/or synthetic oligonucleotides.

Contacting the transduced cells may be carried out by cell surface labeling or by intracellular antigen staining. In accordance with this embodiment, labeling molecules may be antibodies. As used herein, the term “antibody” or “antibodies” refers to any specific binding substance(s) having a binding domain with a required specificity including, but not limited to, antibody fragments, derivatives, functional equivalents, and homologues of antibodies, including any polypeptide comprising an immunoglobulin binding domain, whether natural or synthetic, monoclonal or polyclonal. Chimeric molecules comprising an immunoglobulin binding domain, or equivalent, fused to another polypeptide are also included.

In one embodiment, the labeling molecule comprises a fluorophore. Suitable non-protein organic fluorophores are well known in the art and include, but are not limited to, xanthene, cyanine, squaraine, naphthalene, coumarin, oxadiazole, anthracene, pyrene, oxazine, acridine, arylmethine, tetrapyrrole, and derivatives thereof.

Exemplary xanthene derivatives include, but are not limited to, fluorescein, rhodamine, Oregon green, eosin, and Texas red. Exemplary cyanine derivatives include, but are not limited to, indocarbocyanine, oxacarbocyanine, thiacarbocyanine, and merocyanine. Exemplary squaraine derivatives include, but are not limited to, Seta, SeTau, and Square dyes and naphthalene derivatives (dansyl and prodan derivatives). Suitable coumarin derivatives include, but are not limited to, oxadiazole derivatives: pyridyloxazole, nitrobenzoxadiazole, and benzoxadiazole. Suitable anthracene derivatives include, but are not limited to, anthraquinones, including DRAQ5, DRAQ7, and CyTRAK Orange. Suitable pyrene derivatives include, but are not limited to, cascade blue. Suitable oxazine derivatives include, but are not limited to, Nile red, Nile blue, cresyl violet, and oxazine 170. Suitable acridine derivatives include, but are not limited to, proflavin, acridine orange, and acridine yellow. Suitable arylmethine derivatives include, but are not limited to, auramine, crystal violet, and malachite green. Suitable tetrapyrrole derivatives include, but are not limited to, porphin, phthalocyanine, bilirubin.

When the labeling molecules comprise a fluorophore, the method may further involve exciting the fluorophore. In such a case, detecting comprises detecting fluorescent emission produced by the excited fluorophore. In accordance with this embodiment, detecting the labeling molecules may be carried out by Fluorescence Activated Cell Sorting (“FACS”) or fluorescence microscopy. Suitable methods for FACS and fluorescence microscopy are well known in the art.

In another embodiment, the labeling molecule comprises a metal isotope. Suitable metal isotopes include, but are not limited to, isotopes of lanthanum, cerium, praseodymium, promethium, neodymium, samarium, europium, gadolinium, terbium, dysprosium, holmium, erbium, thulium, ytterbium, and lutetium. The labeling molecule may be a metal-conjugated antibody or antibody fragment.

When the labeling molecules comprise a metal isotope, the method of the present invention further involves ionizing the metal isotope. In this case, detecting comprises detecting the ion cloud produced by the ionized metal isotope. As used herein, the term “CyTOF” or “single cell mass cytometry” refers to the process by which cells labeled with a metal isotope are vaporized to allow the direct analysis of the associated metal isotopes by a time-of-flight mass spectrometer. Thus, in accordance with this embodiment, the detecting step is carried out by cytometry by time-of-flight (“CyTOF”). Suitable methods of CyTOF analysis are well known in the art.

In some embodiments contacting the population of cells with the plurality of vectors is done under conditions effective to achieve a single vector copy per cell. For example, when the vector is a viral vector, cells may be contacted at a low multiplicity of infection (“MOI”). In one embodiment, the MOI is 1 or 0.10.

In other embodiments, the method of the present invention further comprises contacting the transduced cells with a labeling molecule directed to the scaffold protein of each fusion protein. Suitable scaffold proteins are described in detail above.

The method of the present invention may further comprise contacting the cells with a labeling molecule directed to a phenotypic marker. As used herein, the term “phenotypic marker” refers to a property that is determined at the protein level and may be used to characterize a cell. In some embodiments, the method further comprises contacting the transduced cells with labeling molecules capable of binding a phenotypic marker. The method may further involve evaluating phenotypic differences among the transduced cell population, such as determining differences in endogenous protein expression.

The method of the present invention may also comprise contacting the transduced cells with labeling molecules capable of binding the scaffold protein.

In one embodiment, the method of the present invention further involves contacting the transduced cells with labeling molecules capable of binding the transcripts of the fusion protein. In accordance with this embodiment, the method involves detecting specific RNA transcripts.

In accordance with this embodiment, the Pro-Codes are detected in cells by in situ hybridization of Pro-Code encoding RNA with fluorophore-labeled or metal-conjugated nucleic acid probes that bind to the Pro-Code RNA in the cell. Each probe may be specific for a sequence of DNA encoded in the vector which is expressed by an RNA polymerase II or RNA polymerase III promoter. The fluorophore-labeled or metal-conjugated probes may be detected in cells by FACs or CyTOF.

In accordance with this aspect of the invention, the method may be used to track a transduced vector. For example, detecting the labeling molecules to track the transduced cells enables the identification of the transduced vector.

A further aspect of the invention relates to a kit comprising a library of vectors comprising the nucleic acid molecule of the present invention, where each vector comprises a different series of two or more distinct epitopes. Each of the vectors may comprise the same or different effector molecules. As described above, the vectors may be viral vectors. In one embodiment, the vectors are each lentiviral vectors.

Another aspect of the invention relates to a vector encoding a series of two or more distinct RNA sequences, where the distinct two or more RNA sequences are recognized by distinct nucleic acid probes. In one embodiment, the series of two or more distinct RNA sequences are operably linked to a promoter. Various suitable promoters are described in detail above.

Another aspect of the invention relates to a method of tracking a cell. This method involves providing a plurality of vectors according to the present invention, where the vectors encode two or more distinct RNA sequences; providing a population of cells; contacting the population of cells with the plurality of vectors under conditions effective for transduction; contacting the transduced cells with nucleic acid probes capable of binding the two or more distinct nucleic acid sequences of each of the plurality of vectors; and detecting the nucleic acid probes to track the transduced cells.

Suitable vectors, cells, and methods of detecting are described in detail above.

In one embodiment, the two or more distinct nucleic acid sequences are heterologous to the population of cells.

In certain embodiments, vectors may comprise 2, 3, 4, 5, 6, 7, 8, 9, or 10 distinct nucleic acid sequences, each recognized by a distinct nucleic acid probe. The nucleic acid probe may be a DNA probe or an RNA probe.

In one embodiment, the nucleic acid probes comprise a fluorophore. Suitable fluorophores are described above. When the labeling molecules comprise a fluorophore, the method may further involve exciting the fluorophore.

In another embodiment, the nucleic acid probes are conjugated to a metal isotope. Suitable metal isotopes are described above. When the labeling molecules comprise a metal isotope, the method of the present invention further involves ionizing the metal isotope.

The present invention can be used in many applications in which protein reporters or DNA barcodes are used, including vector tracking and cell tracking. The present invention may also be used to track individual cells in a population to determine the behavior of particular cells and cell clones under various conditions (Lu et al., “Tracking Single Hematopoietic Stem Cells In Vivo Using High-Throughput Sequencing in Conjunction with Viral Genetic Barcoding,” Nat. Biotechnol. 29:928-934 (2011) and Bhang et al., “Studying Clonal Dynamics in Response to Cancer Therapy Using High-Complexity Barcoding,” Nat. Med. 21:440-8 (2015), which are hereby incorporated by reference in its entirety). A difference between the vector tracking application is that cell tracking does not involve forced gene modulation. Instead, it can be used for applications such as studying how individual cancer cells respond and resist to a drug. Table 4 below lists various advantages of the technology of the present invention compared to DNA barcoding technology.

TABLE 4
Comparison of DNA Barcodes to the Present Invention.

| DNA Barcodes | Present Invention |
|---|---|
| Cannot phenotype cells. | Multiparameter phenotyping is |
| | possible. |
| Limited primarily to screening | Enables screening for genes that |
| for genes that impact cell | impact numerous aspects of cell |
| fitness (i.e., cell proliferation | biology (any phenotype that can |
| or cell death). | be assessed by flow cytometry, |
| | including cell activation, cell |
| | metabolism, cell cycle, apoptosis, |
| | proliferation). |
| Analysis is made on bulk cell | Analysis is made on individual |
| populations. | cells, and thus provides single |
| | cell resolution. |
| Analysis requires cells to be killed | Cells can be kept alive for analysis |
| (as a result of DNA extraction), and | and put back in culture or in |
| thus analysis is endpoint. This also | animal. This means longitudinal |
| means cells carrying a particular | analysis is possible. It also means |
| DNA barcode cannot be isolated and | cells carrying a specific Pro-Code |
| further used for experimentation. | can be isolated, and used for |
| | further studies (e.g., re-expanded |
| | in culture, injected in mice, etc.) |
| Time consuming and laborious to | Relatively quick to prepare |
| read the barcodes. Requires DNA | samples. Cells are washed and |
| extraction from cells (1 hour), | stained with antibodies (2 hours), |
| preparation of libraries for DNA | and analyzed by FACS or CyTOF |
| sequencing (1-2 days), sequencing, | (1 hour). |
| and analysis (1-2 days). | |

The technology of the present invention is novel in concept and application. It is the first time combinations of epitopes have been used as a cellular barcoding system. The combinatorial approach enables detection of many unique entities (barcodes) with relatively few detection channels. In terms of application, Pro-Codes of the present invention enable high-content phenotyping (>30 different parameters) at the protein level and at single-cell resolution, because these genetic barcodes can be detected by FACS and CyTOF. As shown in the Examples that follow, the Pro-Code technology of the present invention enables the simultaneous identification of a plurality of vectors, each encoding a different effector molecule (e.g., CRISPR gRNA).

The present invention may be further illustrated by reference to the following examples.

### EXAMPLES

### Materials and Methods for Examples 1-6

Mice.

BALB/c and BALB/c Rag1^{-/-} mice were purchased from Jackson Laboratory. Jedi mice (Agudo et al., “GFP-Specific CD8 T Cells Enable Targeted Cell Depletion and Visualization of T-Cell Interactions,” Nat. Biotechnol. 33:1287-1292 (2015), which is hereby incorporated by reference in its entirety) were from established colonies. All mice were hosted in a specific pathogen-free facility. At the time of experimentation, mice were 8-12 weeks of age.

Cell Culture.

293T cells were grown in IMDM with 10% heat-inactivated FBS (Gibco), 100 U/ml penicillin/streptomycin (Gibco), and 2 mM L-Glutamine. Cells were passaged up to 20 times (washed with PBS, detached from the plate with 0.05% Trypsin-EDTA (Gibco), and replated). Cells were discarded after 20 passages. THP-1 were grown in DMEM with 10% heat-inactivated FBS (Gibco), 100 U/ml penicillin/streptomycin (Gibco), 2 mM L-Glutamine, and 55 μM 2-mercaptoethanol. Jurkat cells were grown in RPMI with 10% heat-inactivated FBS (Gibco), 100 U/ml penicillin/streptomycin (Gibco), and 2 mM L-Glutamine. Cells were maintained at a maximum concentration of 1 million per ml. Both Jurkat and THP-1 cells were maintained at a maximum concentration of 1 million per ml. 4T1 cells are a BALB/c cell line of mammary carcinoma. They were cultured in RPMI with 10% heat-inactivated FBS, 100 U/ml penicillin/streptomycin, and 2 mM L-Glutamine. Cells were kept at a maximum confluency of 70% and passaged up to 20 times as described for 293T cells. All cell lines were purchased from ATCC.

Vector Construction.

Linear epitope sequences were cloned into lentiviral vector downstream of the human EF1a promoter in the C terminal region of the dNGFR cDNA using ShpI and BsrGI restriction sites. The Pro-Code vector also contained a U6 gRNA expression cassette similar to the one present in pX330 plasmid (Cong et al., “Multiplex Genome Engineering Using CRISPR/Cas Systems,” Science 339:819-823 (2013), which is hereby incorporated by reference in its entirety). BbsI sites were present downstream of the U6 promoter and upstream of the Cas9 gRNA scaffold for efficient gRNA cloning. Linear epitope sequences were codon-optimized to facilitate expression in mammalian cell systems, organized in combinations of 3, and separated by a flexible linker comprised of six glutamines. Amino acid and nucleotide sequences of all epitope tags are provided in Table 5. To clone gRNA sequences, Pro-Code vectors were digested with BbsI, purified using PCR purification kit (Qiagen), and ligated with pairs of annealed oligo sequences (forward oligo design: 5′ CACCG(N)₂₀; reverse oligo design: 5′ AAAC(N)₂₀C, where (N)₂₀ is the sequence of guide RNA or its reverse complement counterpart). sgRNA sequences were obtained from Brunello (human) or Brie (mouse) CRISPR libraries (Doench et al., “Optimized sgRNA Design to Maximize Activity and Minimize Off-Target Effects of CRISPR-Cas9,” Nat. Biotechnol. 34:1-12 (2016), which is hereby incorporated by reference in its entirety). TOP10 competent cells were used for all subsequent plasmid preparations with exception of lentiCRISPR v2 (Addgene plasmid no. 52961) (Samjana et al., “Improved Vectors and Genome-Wide Libraries for CRISPR Screening,” Nat. Methods 11:783-784 (2014), which is hereby incorporated by reference in its entirety), which was propagated using NEB stable competent cells (New England BioLabs). All plasmids were purified using ZR Plasmid Miniprep Classic kit (Zymo Research) or EndoFree Plasmid Maxi Kit (Qiagen).

TABLE 5
Epitopes

| | | | Amino | SEQ |
|---|---|---|---|---|
| Symbol | | Amino Acid | Acid | ID |
| Used | Name | Sequence | Quantity | NO: |
| E1 | HA | YPYDVPDYA | 9 | 21 |
| E2 | V5 | GKPIPNPLLGLDST | 14 | 24 |
| E3 | S1 | NANNPDWDF | 9 | 27 |
| | (Strep I) | | | |
| E4 | E | GAPVPYPDPLEPR | 13 | 28 |
| E5 | VSVg | YTDIEMNRLGK | 11 | 23 |
| E6 | NWS | NWSHPQFEK | 9 | 30 |
| | (Strep II) | | | |
| E7 | E2 | GVSSTSSDFRDR | 12 | 29 |
| E8 | AU1 | DTYRYI | 6 | 25 |
| E9 | AU5 | TDFYLK | 6 | 26 |
| E10 | FLAG | DYKDDDDK | 8 | 22 |

Pro-Code/CRISPR Libraries.

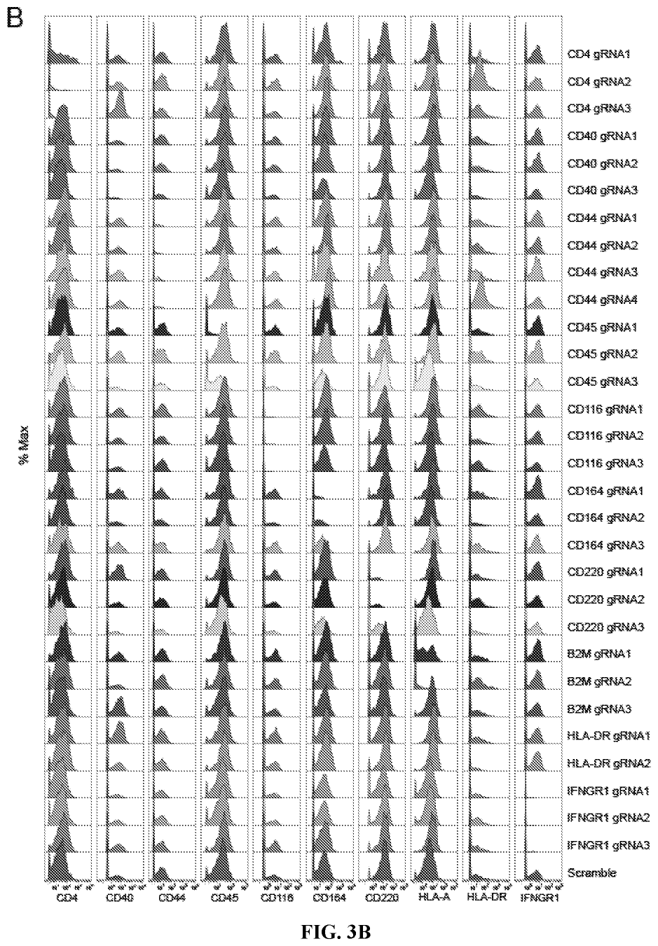

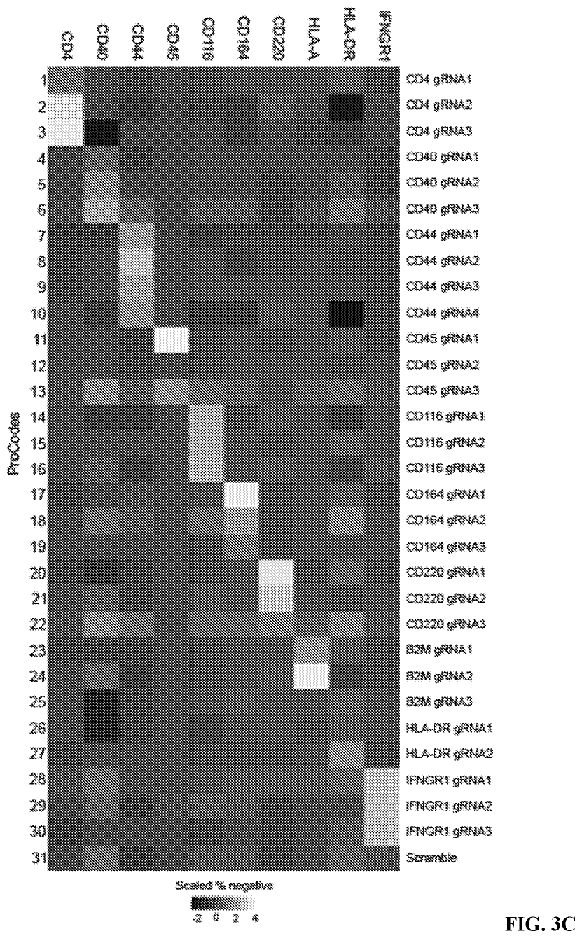

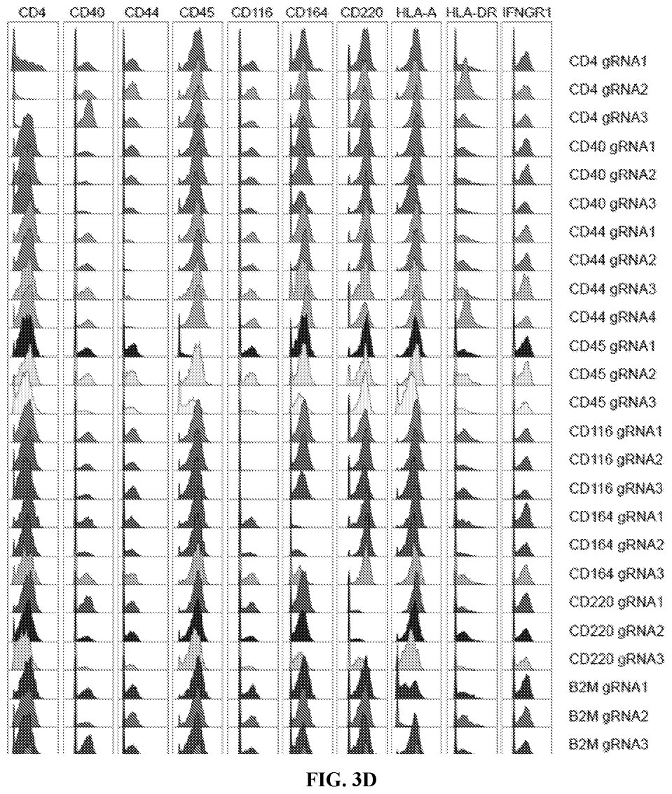

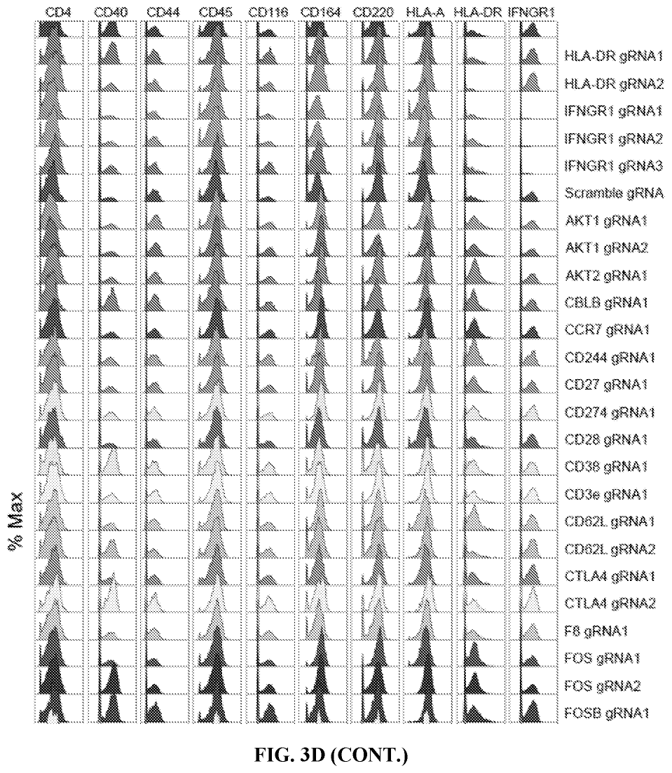

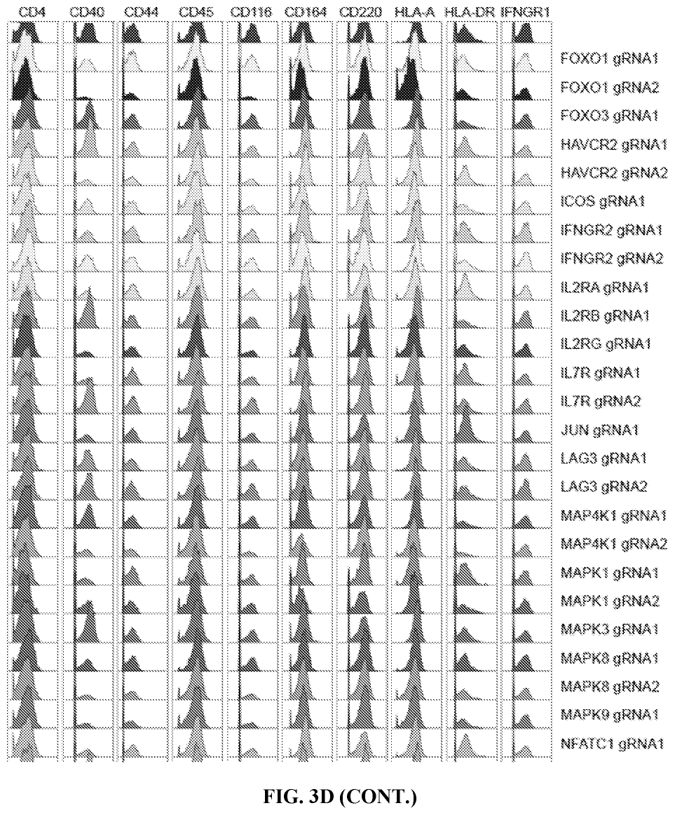

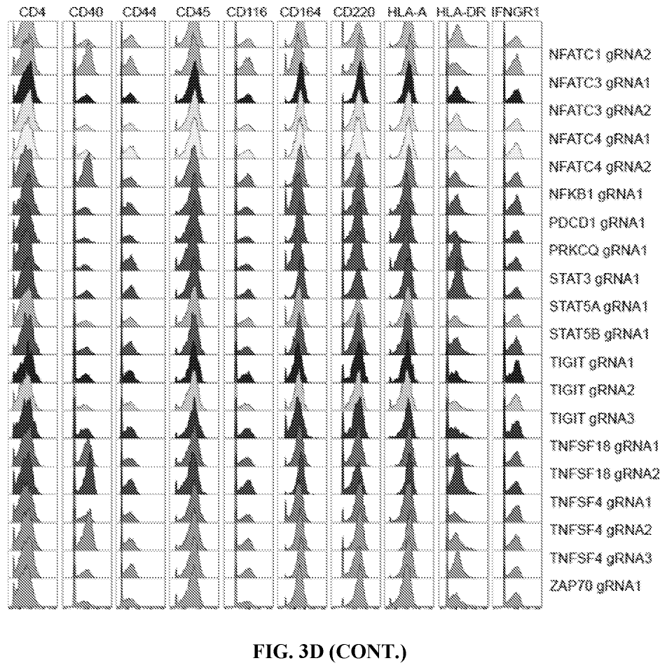

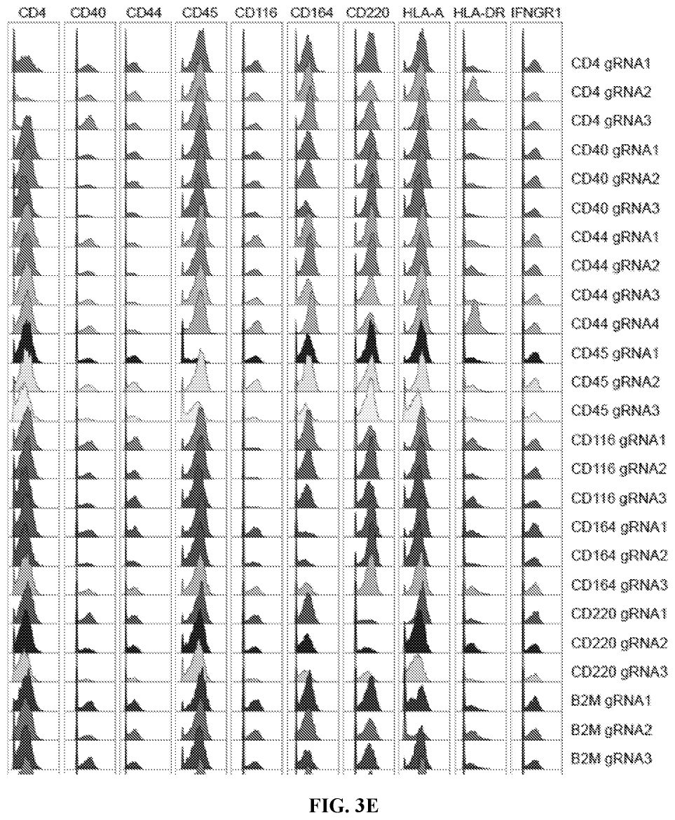

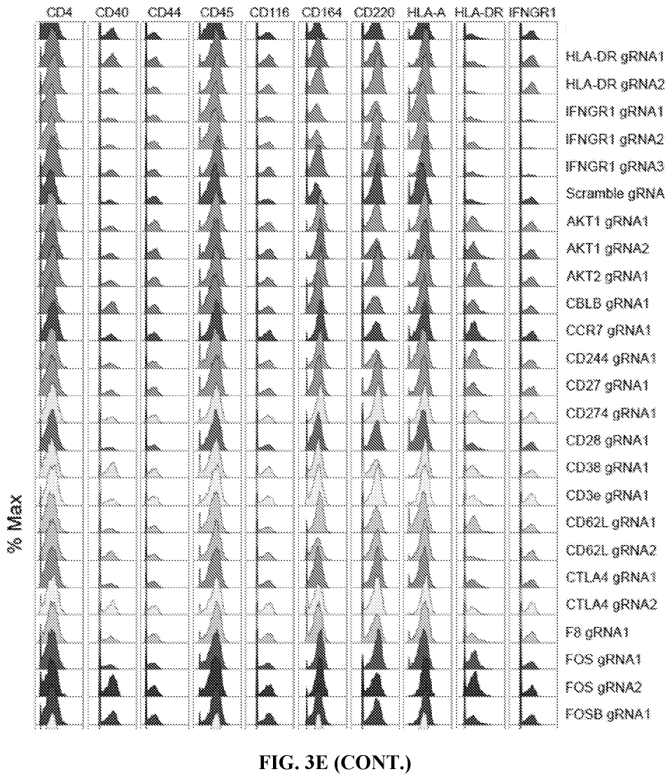

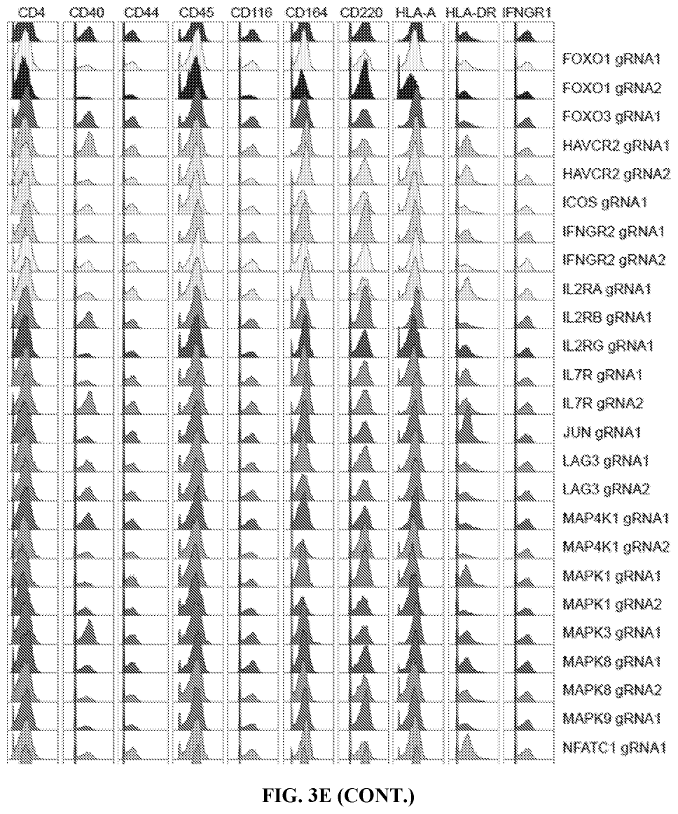

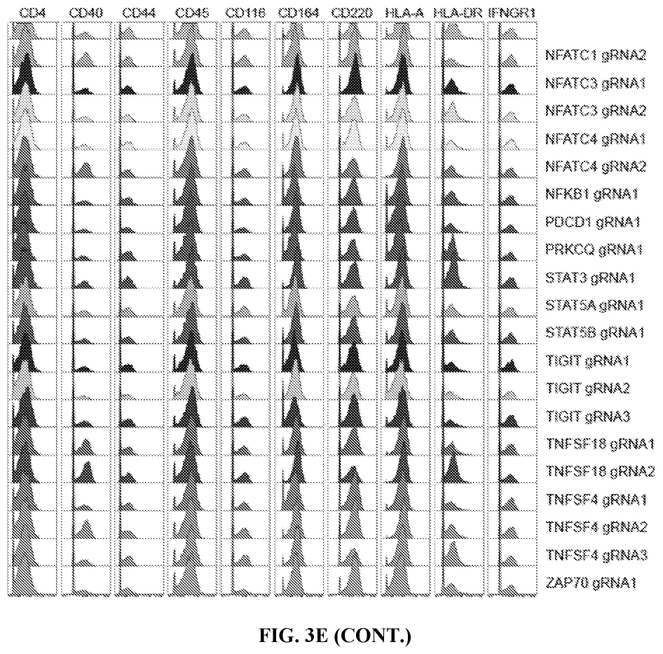

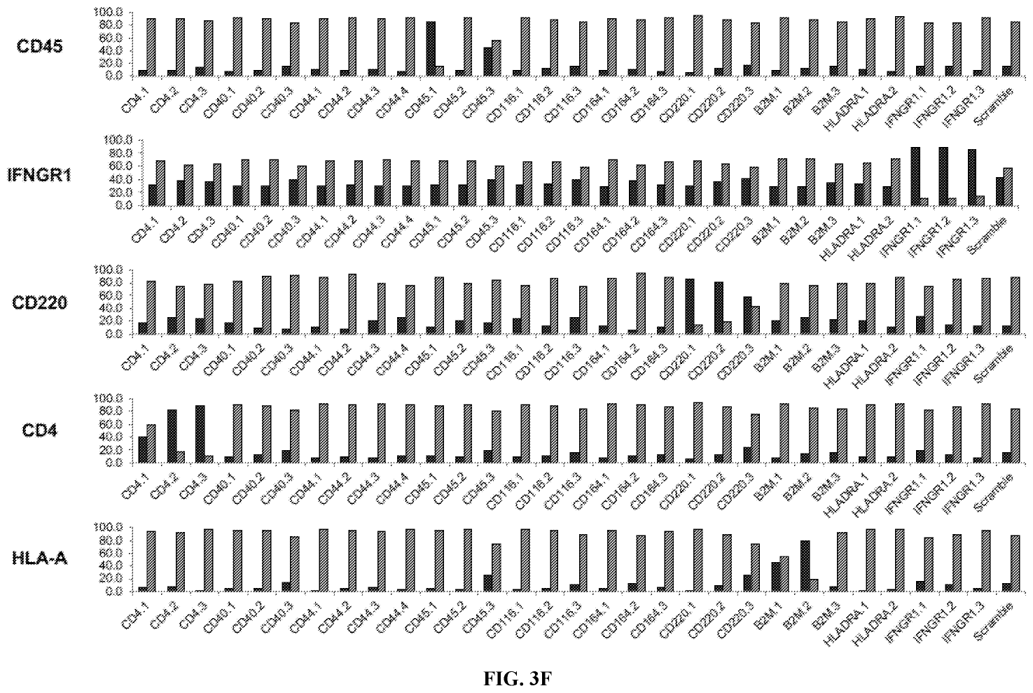

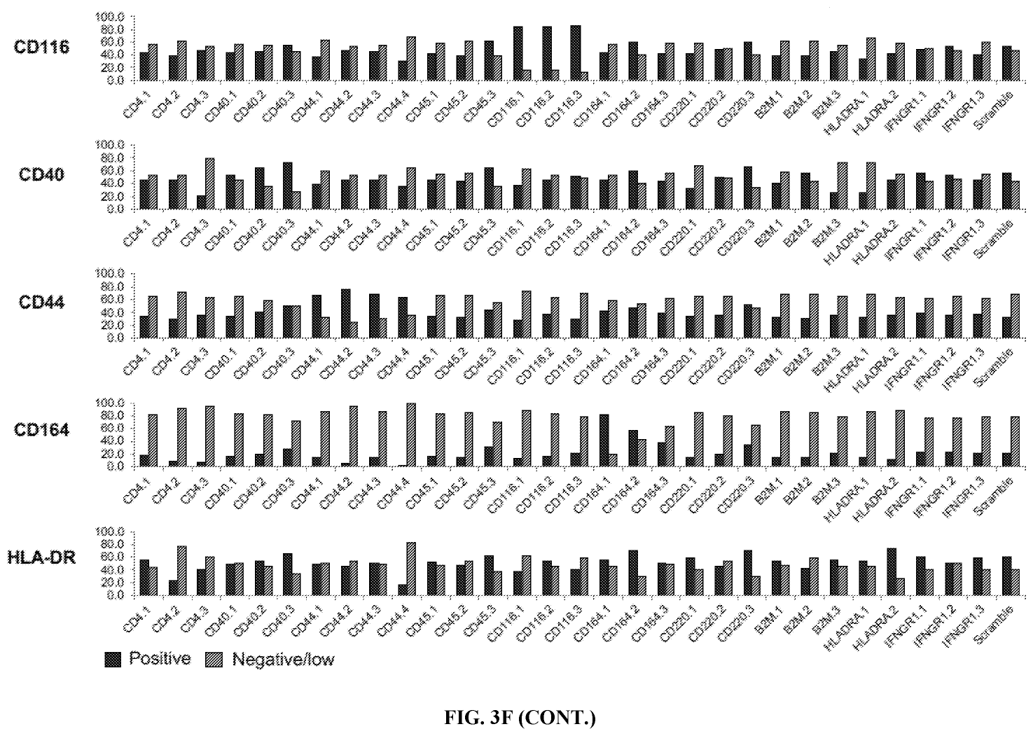

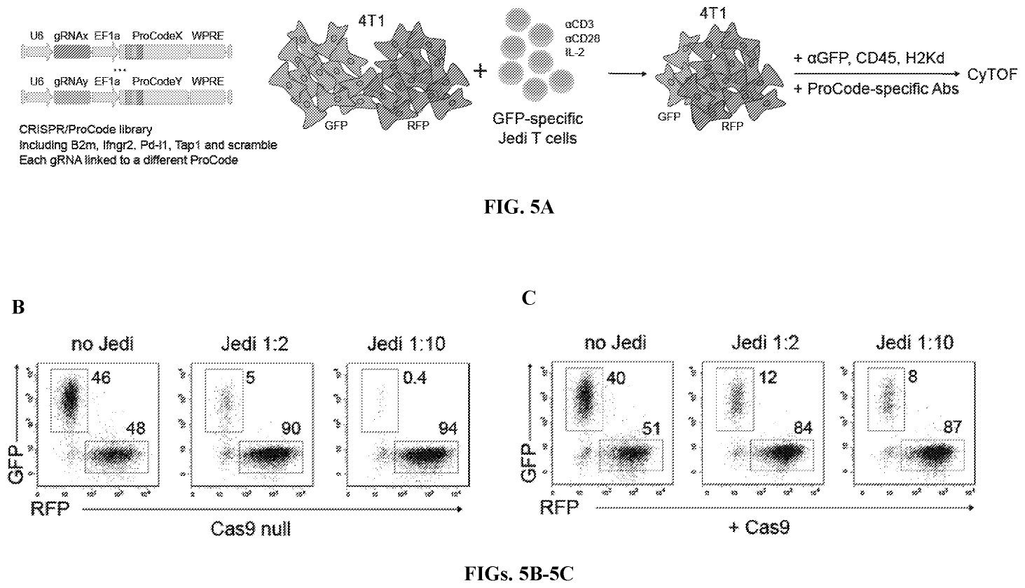

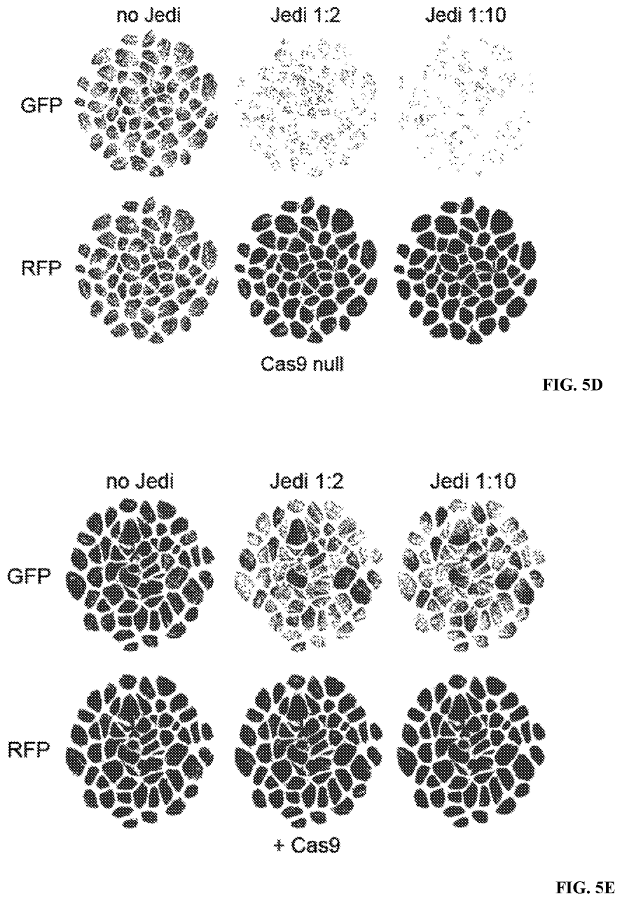

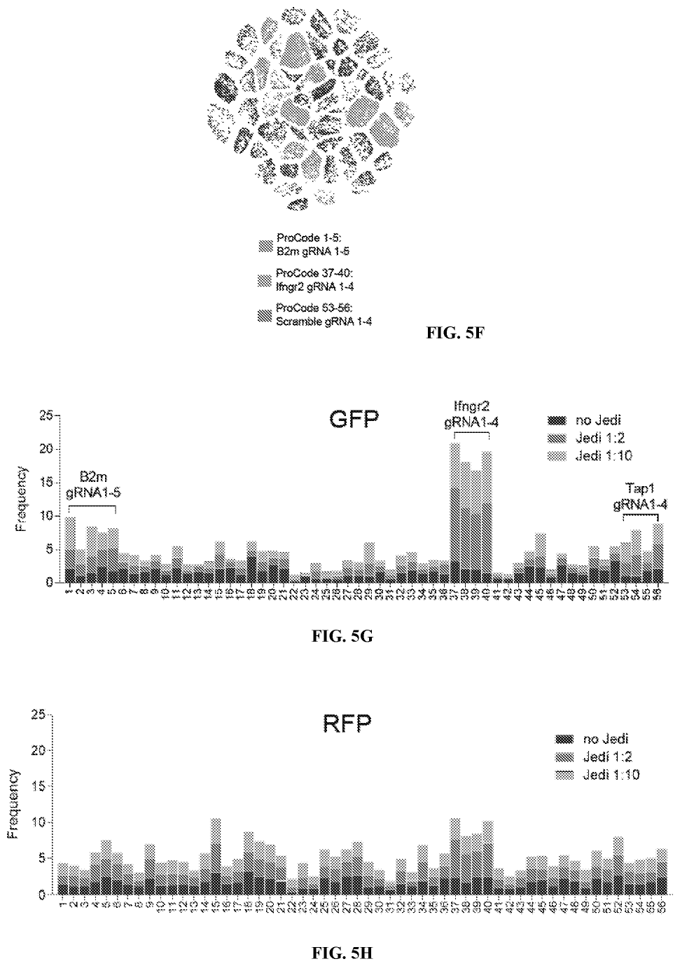

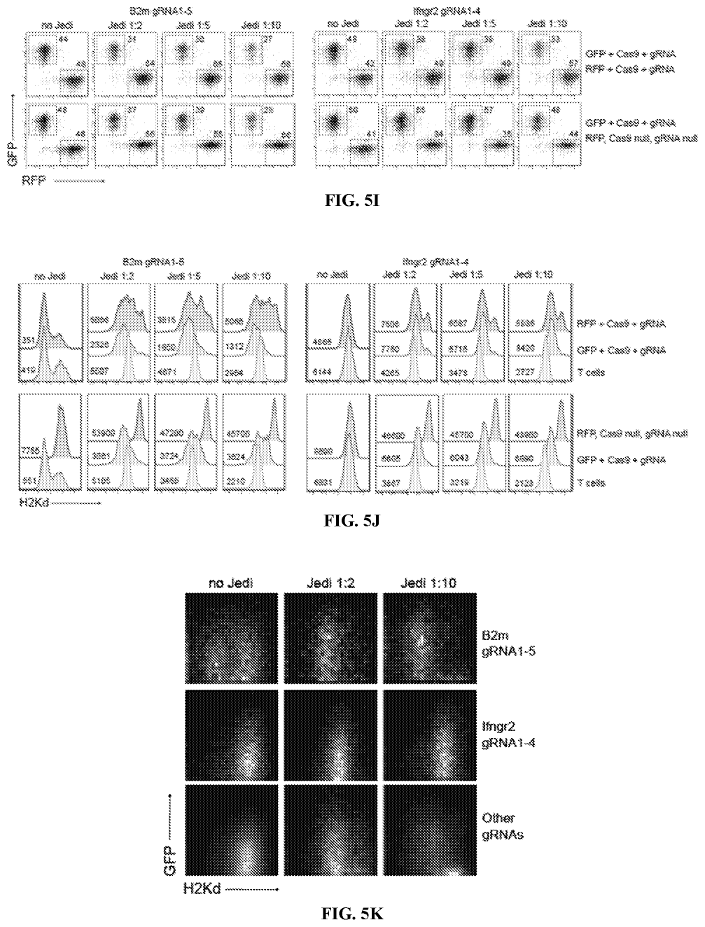

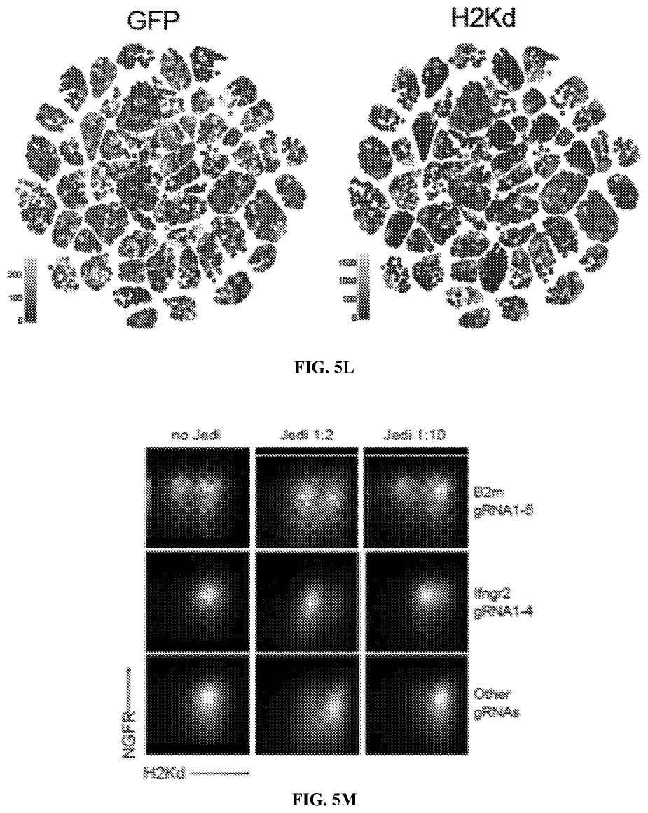

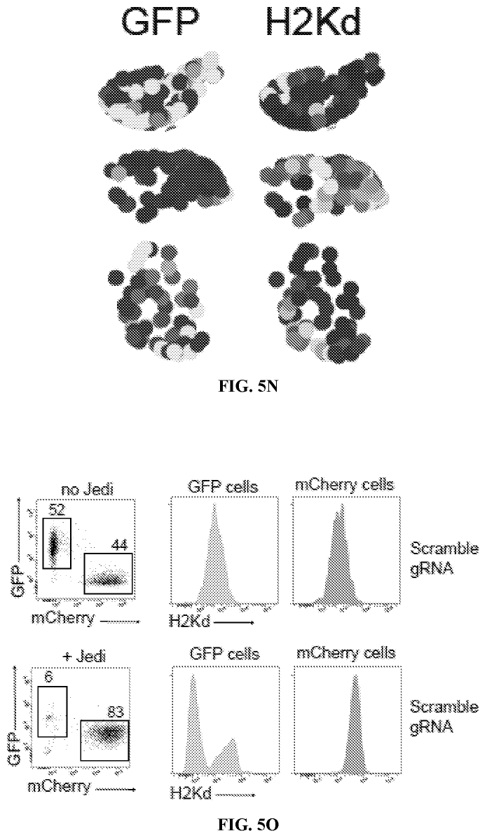

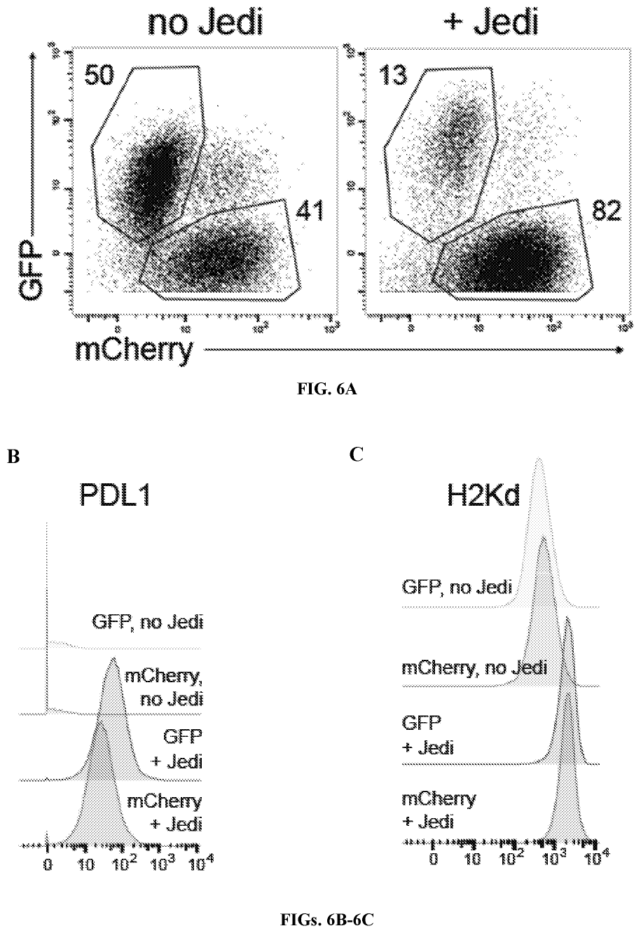

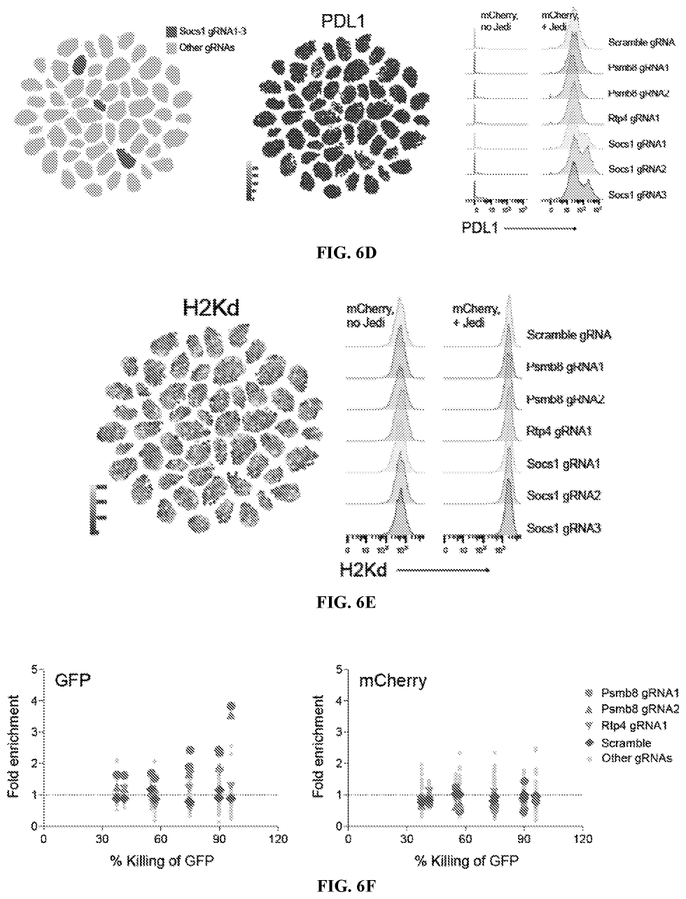

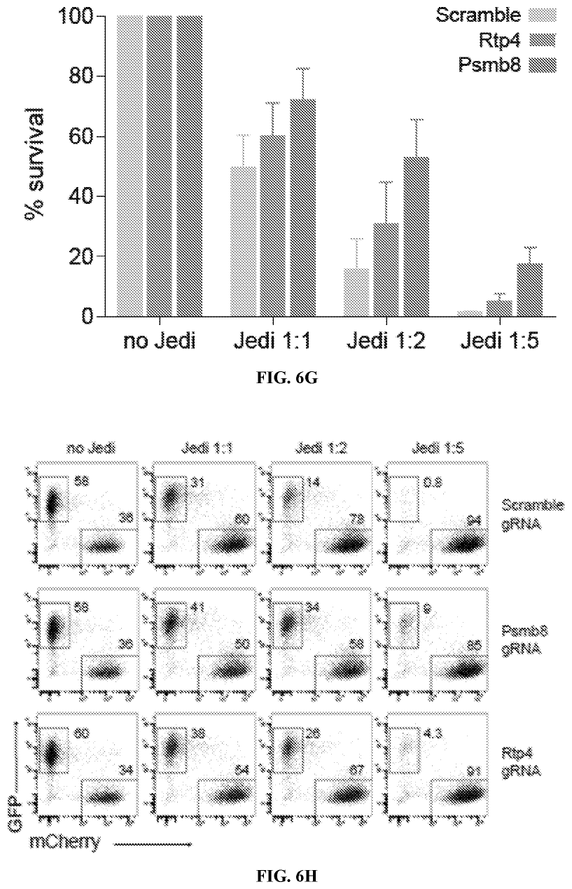

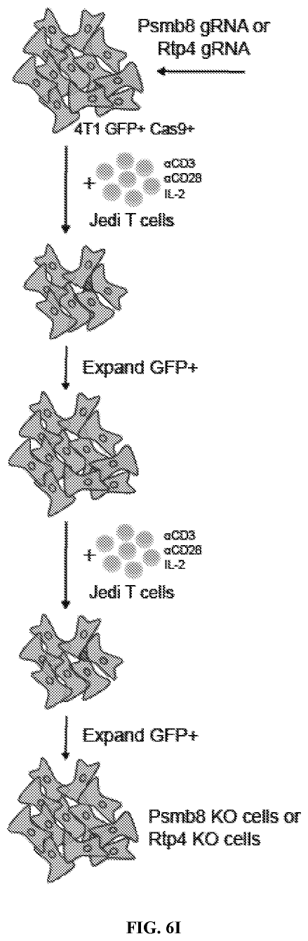

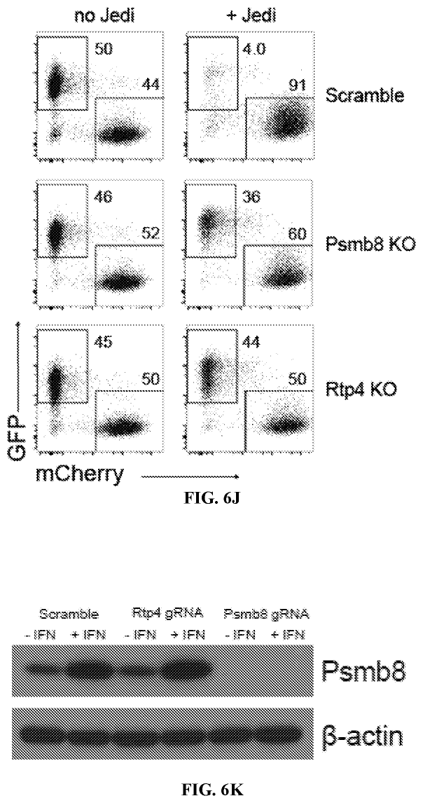

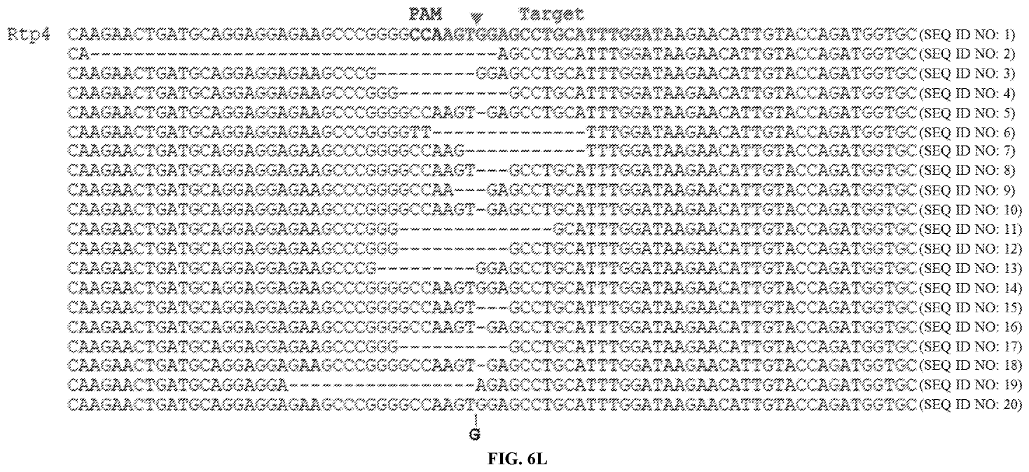

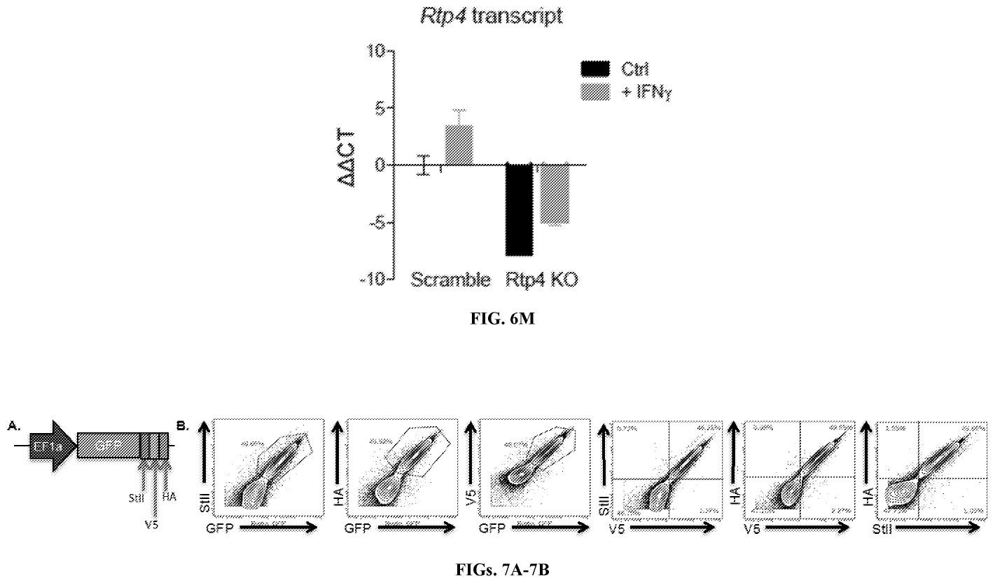

The following genes were targeted in the Pro-Code CRISPR library used in FIGS. 3A-3F: B2M, CD116, CD164, CD220, CD4, CD40, CD44, CD45, HLADRA, IFNGR1, AKT1, AKT2, CBLB, CCR7, CD244, CD27, CD274, CD28, CD38, CD3E, CD62L, CTLA4, F8, FOS, FOSB, FOXO1, FOXO3, HAVCR2, ICOS, IFNGR2, IL2RA, IL2RB, IL2RG, IL7R, JUN, LAG3, MAP4K1, MAPK1, MAPK3, MAPK8, MAPK9, NFATC1, NFATC3, NFATC4, NFKB1, PDCD1, PRKCQ, STAT3, STAT5A, STAT5B, TIGIT, TNFRSF18, TNFRSF4, and ZAP70. The following genes were targeted in the Pro-Code CRISPR library used in FIGS. 5A-50: B2m, Tap1, H2-D1, Pd-11, Fak, Ccr4, Nlrc5, Cxcr7, Cd40, Ifngr2, Cldn4, Ephb2 and H2-Ke6. The following genes were targeted in the Pro-Code CRISPR library used in FIGS. 6A-6L: Socs1-7, Ptpn1, Ptpn2, Rtp4, Rab5b, Stip1, Supt16, and Psmb8.

Lentiviral Vector Production and Titration.

Lentiviral vectors were produced as previously described in detail (Baccarini et al., “Kinetic Analysis Reveals the Fate of a MicroRNA Following Target Regulation in Mammalian Cells,” Curr. Biol. 21:369-376 (2011), which is hereby incorporated by reference in its entirety). Briefly, 293T cells were seeded 24 hours before calcium phosphate transfection with third-generation VSV-pseudotyped packaging plasmids and the transfer plasmids. Supernatants were then collected, passed through a 0.22-μm filter, purified by ultracentrifugation, aliquoted, and stored at −80° C. Viral titer was estimated on 293T cells by limiting dilution. LentiCRISPR v2 transfer plasmid encoding Cas9 transgene and a puromycin resistant cassette was used to generate Cas9 lentivirus. To produce LV Pro-Code libraries, equimolar amounts of single plasmids were pooled and subsequently used for vector production. Alternatively, each LV was produced individually in a 96-well format, and all LVs were pooled in equimolar ratio before transduction. Where indicated, the Pro-Code libraries were co-transfected with pCCLsin.PPT.hPGK.GFP at 50% of total transfer plasmids.

Vector Transduction.

293T, THP-1, Jurkat, and 4T1 cells were transduced as previously described (Mullokandov et al., “High-Throughput Assessment of MicroRNA Activity and Function Using MicroRNA Sensor and Decoy Libraries,” Nat. Methods 9:840-846 (2012), which is hereby incorporated by reference in its entirety). To ensure that a majority of transduced cells received only one vector, fewer than 10% of cells were transduced in all experiments. For knockout experiments, THP1, Jurkat, and 4T1 cells were engineered to stably express Cas9. Briefly, cells were seeded 24 hours prior to transduction in 6-well plates at 5×10⁴ cells per well, and transduced with Cas9 lentivirus in the presence of 5 μg/ml polybrene (Millipore). 48 hours after transduction, cells were treated overnight with 10 μg/ml puromycin (ThermoFisher) to remove all non-transduced cells. Puromycin treatment was repeated two additional times to ensure cell purity. Cas9 expression was confirmed by western blot using anti-Cas9 antibody (Millipore, clone 7A9). For T-cell killing experiments, 4T1 cells (+/−Cas9) were first transduced with GFP, iRFP670 or mCherry lentiviral vectors, then with Pro-Code/CRISPR libraries.

Flow Cytometry and Cell Sorting.

Before FACS analysis, adherent cells were detached with 0.05% trypsin-EDTA, washed, and resuspended in sterile PBS. Cells grown in suspension were washed and resuspended in sterile PBS. For analysis of NGFR, GFP, or iRFP670 expression, cells were washed and resuspended in flow buffer (PBS, 2 mM EDTA, 0.5% BSA). For immune staining, flow buffer was supplemented either with anti-mouse CD16/CD32 antibody (eBioscience) or Human TruStain FcX Fc Receptor Blocking Solution (BioLegend). Following antibodies were used for flow analysis: anti-human CD271 PE and APC (BD Biosciences), anti-mouse H2Kd PE, Pacific Blue or biotin, anti-mouse B2m PE, anti-mouse CD45 PE-Cy7 (all from eBioscience), streptavidin PE-Cy7 (BioLegend). Data was acquired using BD Fortessa (BD) and analysis was performed using Cytobank (Kotecha et al., “Web-Based Analysis and Publication of Flow Cytometry Experiments,” Curr. Protoc. Cytom. Chapter 10 (2010), which is hereby incorporated by reference in its entirety) or FlowJo Software (FlowJo, LLC). For T-cell killing experiments, transduced 4T1 cells were sorted on a FACS Aria II (BD) to enrich for the NGFR⁺/GFP⁺, NGFR⁺/iRFP670⁺ or NGFR⁺/mCherry⁺ populations.

Tumor Model.

4T1 murine mammary gland carcinoma cells were injected (5·104 cells) in the mammary fat pad of 8-12 week old BALB/c WT or Rag1^{-/-} mice. Tumor-inoculated mice were sacrificed 14 days later. Tumor cell suspensions were obtained by enzymatic treatment with RPMI supplemented with collagenase (1.5 mg/ml) and BSA (25 mg/ml) (45 min at 37° C.). Digested tumors were homogenized by multiple passage through a 19G needle and filtered twice through a 40-μm cell strainer. Cells were put in culture with 6-thioguanine (60 μM) for 3 days to enrich for 4T1 cells, and remove stromal cells (hematopoietic, fibroblast, and endothelial) so that they would not be part of the cellular mixture analyzed. 3×10⁶ cells per tumor were analyzed for Pro-Code distribution by CyTOF.

T-Cell Killing Assay.

CD8⁺ T-cells were isolated from spleens of Jedi mice. Splenic cell suspensions were obtained by mechanical disruption and filtering through 70-μm cell strainer. Red blood cells were lysed using RBC buffer (eBioscience), and CD8⁺ T-cells were negatively selected using EasySep mouse CD8⁺ T-cells isolation kit from StemCell Technologies, following manufacturer's instructions. Cells were activated for 3 days with 5 μg/ml plate-bound anti-CD3 mAb (clone 2C11, BioXCell), 1 μg/ml anti-CD28 mAb (clone 37.51, BioXCell), and 20 ng/ml mouse recombinant IL-2 (Peprotech) in RPMI with 10% FBS, 100 U/ml penicillin/streptomycin, 2 mM L-glutamine, 1% non-essential amino acids, 1 mM sodium pyruvate 55 μM 2-mercaptoethanol, and 20 mM HEPES. 4T1 cells (+/−Cas9, +/−GFP, +/−iRFP670 (Shcherbakova and Verkhusha, 2013), +/−mCherry) were transduced with the Pro-Code/CRISPR vector pool at a MOI of 1 and cell sorted based on NGFR expression. A 50:50 mix of GFP⁺ (target cells) and either iRFP670⁺ or mCherry (bystander cells) 4T1 cells were plated in 24-well plates (4·10⁴ cells per well). Activated T-cells were added to the wells 6 hours later, at different ratios. Cells were passaged every 2 days and seeded in a 6-well plate at day 2 and in a 10 cm dish at day 6. Killing was assessed by flow cytometry at day 2 and 4. At day 3 or 6, 3·10⁶ cells were stained with the antibodies specific for Pro-Code epitope tags, CD45, H2-Kd, PD-L1, mCherry, and GFP and analyzed by CyTOF.

Mass Cytometry.

Antibodies were either purchased pre-conjugated from Fluidigm or purchased purified and conjugated in-house using MaxPar X8 Polymer Kits (Fluidigm) according to the manufacturer's instructions. The following antibodies were used for CyTOF staining: HA tag-147Sm (clone 6E2, Cell Signaling), V5 tag-152Sm (Thermo Fisher Scientific), anti-DYKDDDDK (FLAG) tag-175Lu (clone 5A8E5, GenScript), VSVg tag-158Gd (rabbit pAb, Thermo Fisher Scientific), E tag-154Sm (clone 10B11, Abcam), E2 tag-160Gd (rabbit pAb, GenScript), NWSHPQFEK (NWS) tag-159Tb (clone 5A9F9, GenScript), S1 tag-153Eu (rabbit pAb, GenScript), AU1-162Dy (clone AU1, BioLegend), AU5-169Tm (clone AU5, BioLegend), H2Kd-biotin or H2Kd-149Sm (clone SF1-1.1.1, eBioscience), αGFP-155Gd (clone FM264G, BioLegend), αmCherry-142Nd (Abcam), anti-mouse CD274-149Sm (MIHS, eBioscience), anti-human CD126-151Eu (clone UV4, BioLegend), anti-human CD119-biotin (eBioscience), phospho STAT1-153Eu (Fluidigm), phospho STAT3 PE (eBioscience), phospho STAT5-150Nd (Fluidigm), anti-PE-165Ho, anti-biotin-143Nd (Fluidigm), anti-mouse CD90.2-113In (Fluidigm), and anti-mouse CD45-141Pr (Fluidigm). Before CyTOF analysis, cells were collected, washed, resuspended in media and stained for viability with Cell-ID Intercalator-103Rh for 15 minutes at 37° C. To avoid non-specific staining, cells were subsequently blocked in flow buffer supplemented with either anti-mouse CD16/CD32 antibody (eBioscience) or Human TruStain FcX Fc Receptor Blocking Solution (BioLegend) for 30 minutes on ice. For phosphorylation experiments, THP1 cells were first labelled with a unique barcode by incubating with CD45-antibodies conjugated to distinct metal isotopes before pooling. Next, cells were stained for cell surface antigens, fixed and permeabilized using BD Cytofix/Cytoperm solution (BD Biosciences), and stained with the tag antibodies for 30 minutes on ice. For phosphorylation experiments, immediately after stimulation cells were incubated with 1% PFA on ice for 20 minutes, washed, and fixed with pure methanol overnight in −80° C. After intracellular/tag staining, cells were washed and incubated in 0.125 nM Ir intercalator (Fluidigm) diluted in PBS containing 2% formaldehyde for 30 min at room temperature, washed, and stored in PBS at 4° C. Immediately prior to acquisition, samples were washed once with PBS, once with de-ionized water, and then resuspended at a concentration of 1·10⁶ per ml in deionized water containing a 1:20 dilution of EQ 4 Element Beads (Fluidigm). The samples were acquired on a CyTOF2 (Fluidigm) equipped with a SuperSampler fluidics system (Victorian Airships) at an event rate of <500 events/second. After acquisition, the data were normalized using bead-based normalization using the CyTOF software. The data were gated to exclude residual normalization beads, debris, dead cells, and doublets, leaving NGFR⁺ events for clustering and high dimensional analyses.

Western Blot.

Rtp4 KO, Psmb8 KO, or control sgRNA-transduced 4T1-Cas9-GFP cells were stimulated with 10 ng/ml IFNγ (Peprotech) for 48 hours. Western blot was performed as previously described (Agudo et al., “The miR-126-VEGFR2 Axis Controls the Innate Response to Pathogen-Associated Nucleic Acids,” Nat. Immunol. 15:54-62 (2013), which is hereby incorporated by reference in its entirety) using rabbit monoclonal anti-Psmb8 antibody (Cell Signaling, clone D1K7X).

qPCR.

Rtp4 KO, Psmb8 KO, or control sgRNA-transduced 4T1-Cas9-GFP cells were stimulated with 10 ng/ml IFNγ (Peprotech) for 48 hours. RNA was extracted from cells using QIAzol Lysis Reagent (Qiagen) according to the manufacturer's instruction. For cDNA synthesis, 1 μg total RNA was reverse-transcribed for 1 hour at 37° C. with an RNA-to-cDNA kit (Applied Biosystems). For quantitative PCR, SYBR green qPCR master mix (Thermo Scientific) and the primers identified in Table 6 below were used.

TABLE 6
qPCR Primers

| | | SEQ |
|---|---|---|
| | | ID |
| Primer | Sequence | NO: |
| mouse Actb | 5′-CTAAGGCCAACCGTGAAAAG-3′ | 61 |
| forward | | |
| mouse Actb | 5′-ACCAGAGGCATACAGGGACA-3′ | 62 |
| reverse | | |
| mouse Rtp4 | 5′-CGGGGCCAAGTGGAG-3′ | 63 |
| forward | | |
| mouse Rtp4 | 5′-TGGCACAAGATCATCACCTG-3′ | 64 |
| f reverse | | |

Sanger Sequencing of the Rtp4 Gene.

To detect CRISPR/Cas9-induced gene editing of the Rtp4 gene, genomic DNA was isolated from cells using DNeasy Blood & Tissue Kit (Qiagen). A 500 bp-size region flanking the target site of the Rtp4 gRNA (5′-ATCCAAATGCAGGCTCCACT-3′ (SEQ ID NO:65)) was PCR amplified using DreamTaq polymerase (Thermo Fisher Scientific) shown in Table 7 below.

TABLE 7
Sequencing Primers

| | | SEQ |
|---|---|---|
| | | ID |
| Primer | Sequence | NO: |
| Forward | 5′-TCTCTCCCAGATTTGAGGAAGA-3′ | 66 |
| primer | | |
| Reverse | 5′-AGCATGGGGACATGGAGTAC-3 | 67 |
| primer | | |

The PCR product was cloned into pCR®4-TOPO® plasmid using TOPO® TA Cloning Kit for Sequencing (Thermo Fisher Scientific) and transformed into TOP10 competent cells. Resulting colonies were then sequenced using M13 forward primer and aligned to the Rtp4 gene in the reference mouse genome.

Data Visualization and Analysis.

CyTOF data was first debarcoded using Single Cell Debarcoder (Zunder et al., “Palladium-Based Mass Tag Cell Barcoding with a Doublet-Filtering Scheme and Single-Cell Deconvolution Algorithm,” Nat. Protoc. 10:316-333 (2015), which is hereby incorporated by reference in its entirety) using post-assignment debarcode stringency filter and outlier trimming. Clean, concatenated files were then visualized using viSNE (Amir et al., “viSNE Enables Visualization of High Dimensional Single-Cell Data and Reveals Phenotypic Heterogeneity of Leukemia,” Nat. Biotechnol. 31:545-552 (2013), which is hereby incorporated by reference in its entirety), a dimensionality reduction method, which uses the Barnes-Hut acceleration of the t-SNE algorithm. viSNE was implemented using either the Rtsne R package or Cytobank (Kotecha et al., “Web-Based Analysis and Publication of Flow Cytometry Experiments,” Curr. Protoc. Cytom. Chapter 10 (2010), which is hereby incorporated by reference in its entirety) and generated using as input tag expression levels transformed by dividing by 5 and taking the arc-sine of the resulting value. Cell clusters were defined either by tag expression or in an unbiased way using the DBSCAN algorithm implementation in R after dimensionality reduction by t-SNE. Heatmaps of cell clusters were generated by taking the median untransformed or arc-sine transformed intensity within clusters and using this value unscaled or Z scaled.

Statistical Analysis.

All statistical details of experiments, including reproducibility (number of independent experiments performed), number of data point per group, and definition of center and dispersion for each group are detailed in the brief description of the drawings above. Heatmaps of cell clusters were generated by taking the median untransformed or arc-sine transformed intensity or the percentage of negative cells within clusters and using this value unscaled or Z scaled relative to other cell clusters.

### Example 1—Pro-Codes Enable Highly Multiplexed Cell Barcoding at the Protein Level

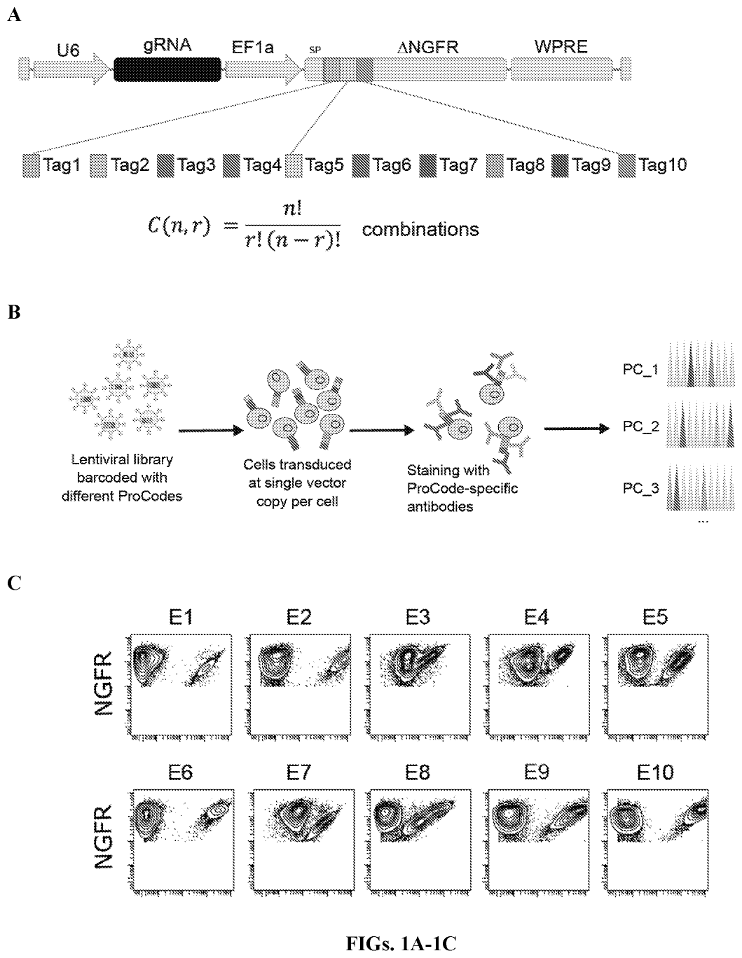

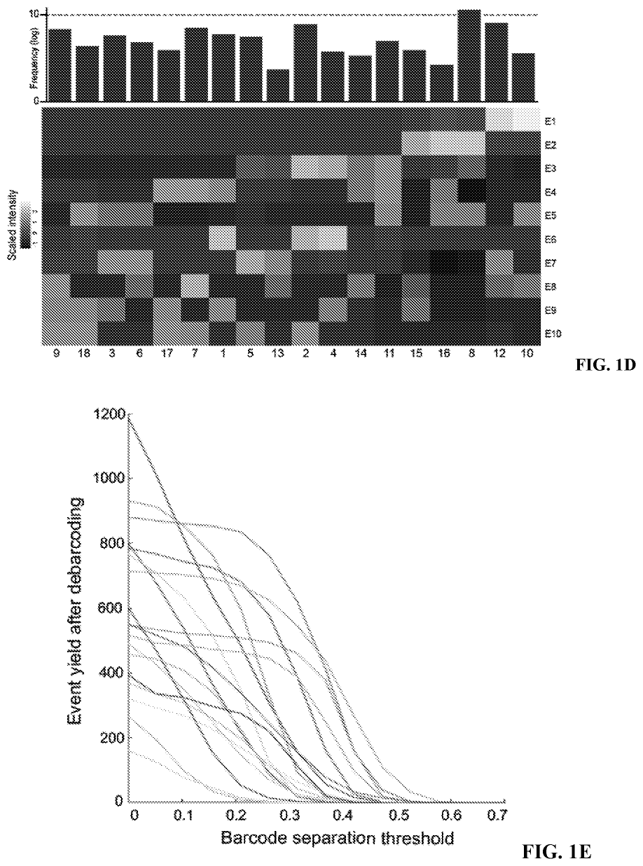

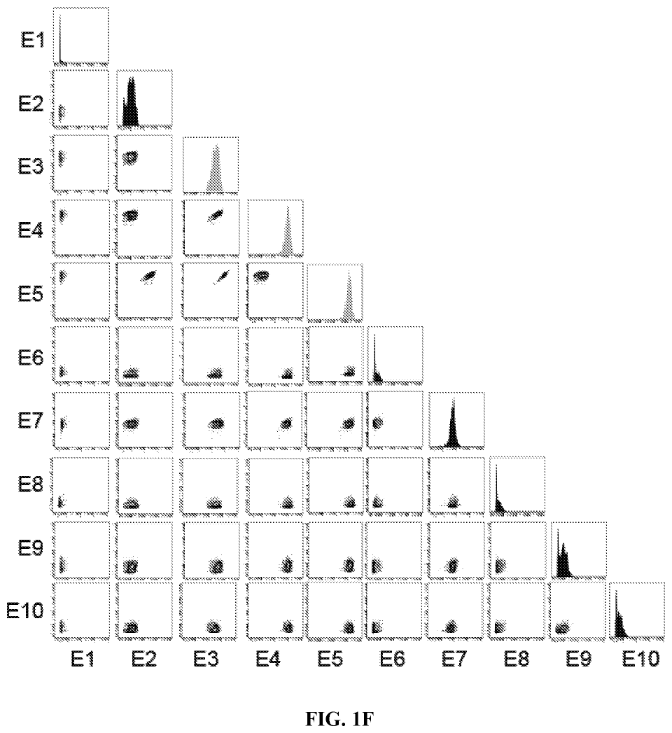

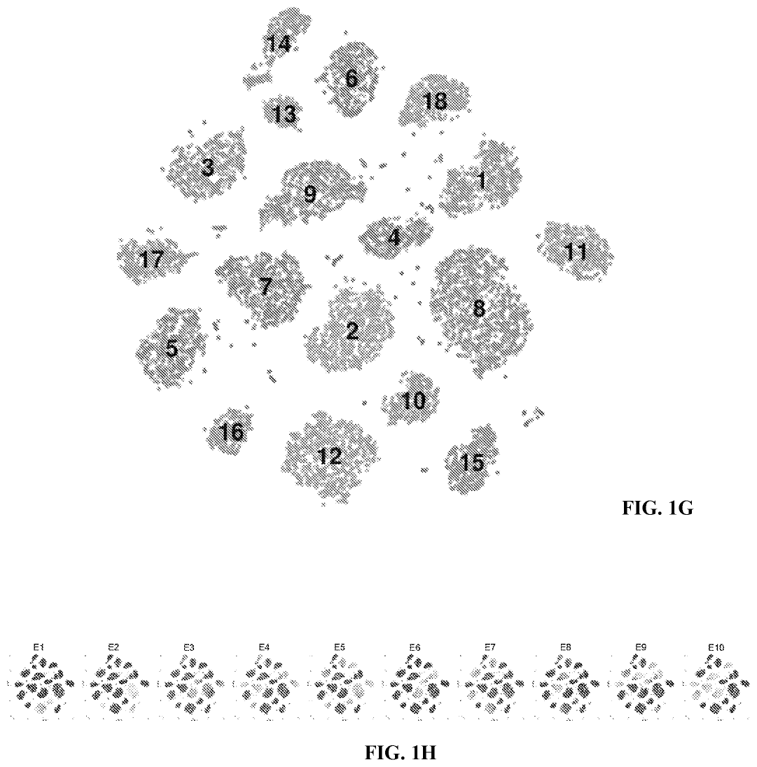

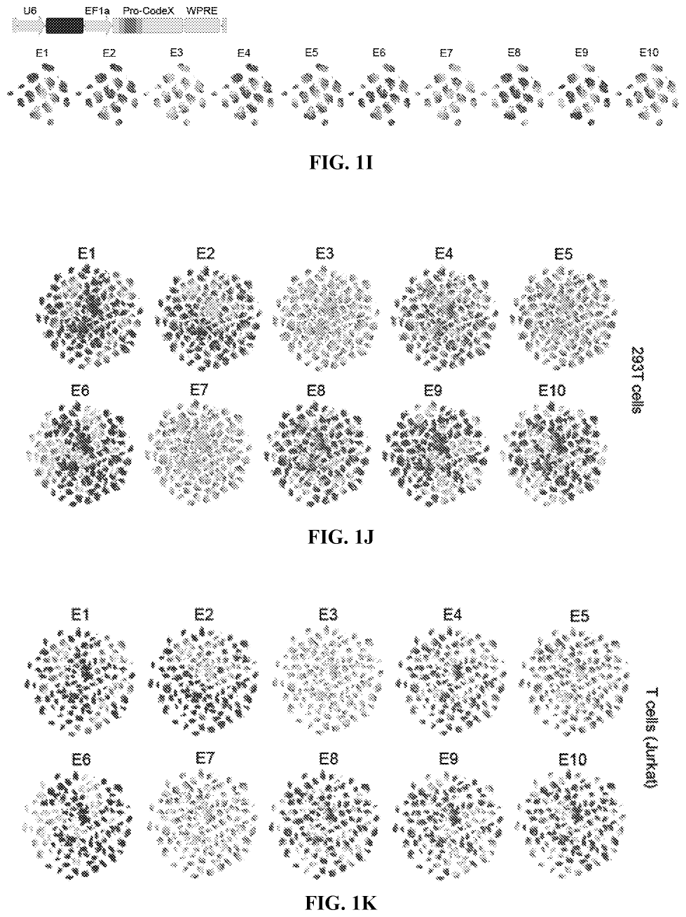

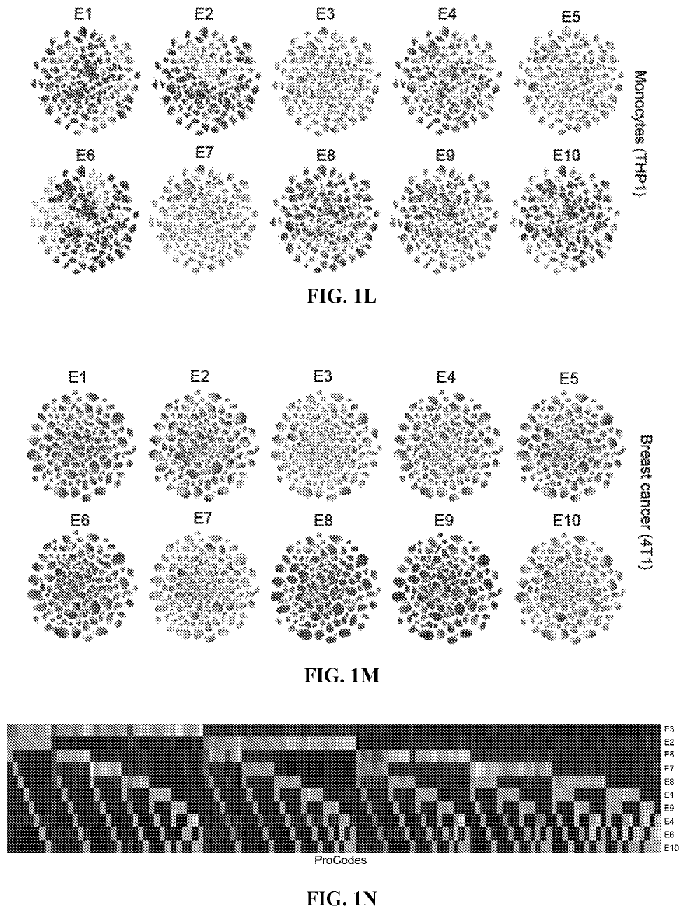

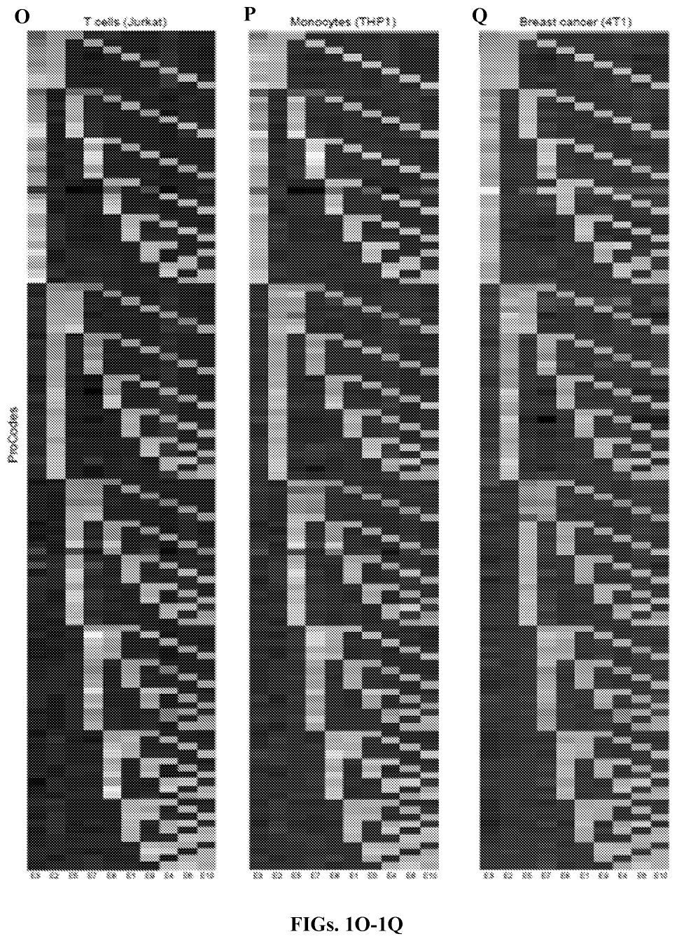

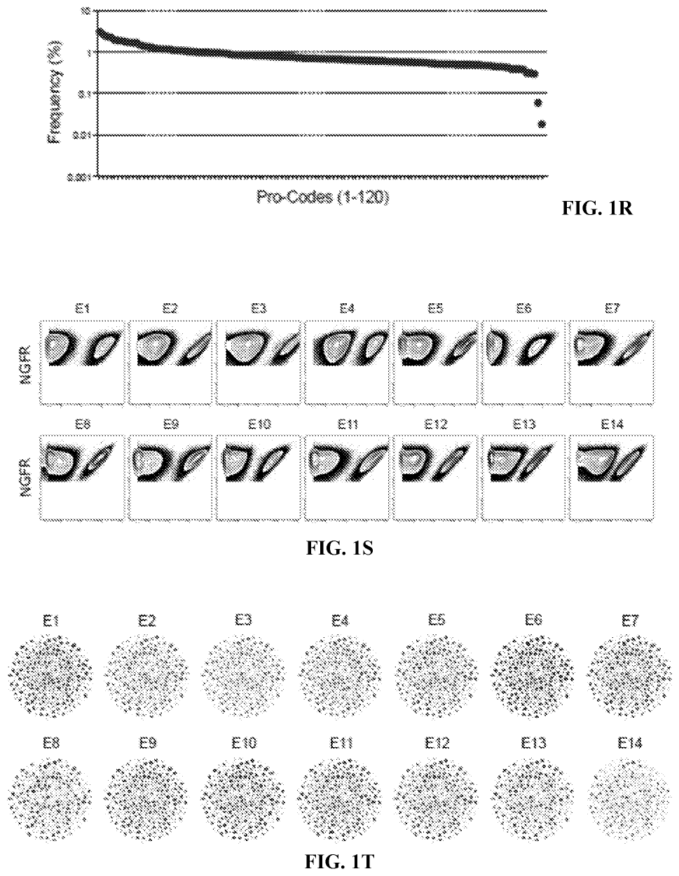

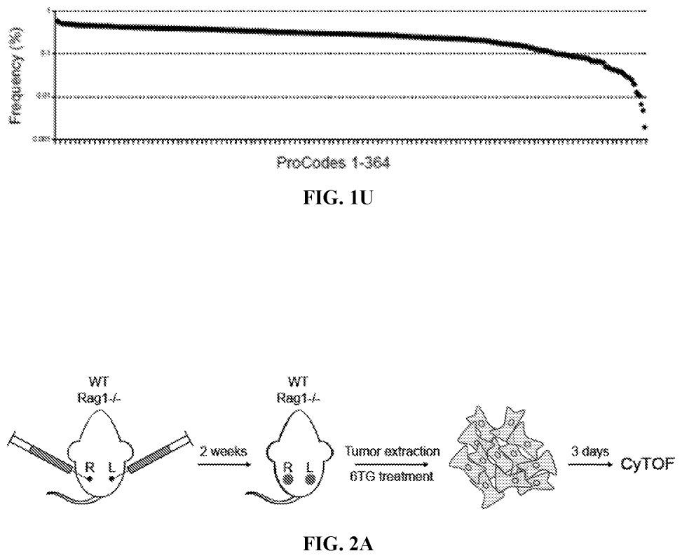

Applicants sought to generate a vector barcoding system that operates at the protein level, as this would enable the ability to multiplex many gene delivery vectors together, detect them in cells using high-throughput, single cell resolution technologies (e.g., flow cytometry), and complex phenotyping. DNA barcodes do not allow this. Reporter proteins (such as GFP and RFP) have the limitation that each protein requires its own detection channel, which limits the number of unique fluorescent reporters that can be used together, generally to 3 or 4, since fluorescent proteins have broad emission spectrums that can overlap. Even with a technology such as mass cytometry (“CyTOF”), this would permit detection of a maximum of 30-40 reporters. It was hypothesized that combinations of a limited number of antibody-detectable epitopes (n) could be arranged together in specific multiples (r) to form a higher order set of barcodes (C) (FIG. 1A). Using this strategy, as few as 10 epitopes could be arranged in sets of 3 to form 120 different combinations (FIG. 1B), and with just 20 epitopes and 7 positions, 77,520 different combinations can be generated. It was further hypothesized that fusing these epitopes onto a protein that is exported to the surface of a cell, such as a receptor, would enable the tags to be detected by antibodies, and analyzed by technologies such as FACS or CyTOF.

Epitopes are fragments of proteins detectable by an antibody. Epitopes can be conformational or linear. Although linear epitopes may be encoded by relatively shorter sequences (e.g., 18-42 nucleotides) and do not require tertiary structure to be detected, conformational epitopes may also be utilized. Ten linear epitopes in which there is an existing antibody for detection were identified. Amongst these were epitopes commonly used as protein tags, such as HA, FLAG, and V5, as well as other epitope/antibody pairs (Table 5 supra). DNA sequences encoding each epitope were synthesized and assembled into every possible unique combination of 3, for a total of 120 different 3-epitope combinations. Each epitope was separated by 6 glutamines that served as a spacer. Each epitope combination was fused to dNGFR, a truncated receptor without an intracellular domain that is commonly used as a reporter protein (Amendola et al., “Coordinate Dual-Gene Transgenesis By Lentiviral Vectors Carrying Synthetic Bidirectional Promoters,” Nat. Biotechnol. 23:108-116 (2005), which is hereby incorporated by reference in its entirety). This was done to provide a scaffold, and to facilitate epitope transport to the cell's surface (FIGS. 1A-1B). The epitopes were inserted after dNGFR signal peptide to preserve dNGFR trafficking to the surface, and ensure the epitopes would be on the extracellular portion of dNGFR. Each of the 120 3-epitope combinations (herein referred to as “Pro-Codes”) fused to dNGFR were cloned in to a lentiviral vector (“LV”) downstream of the human EF1a promoter.

To determine if cells expressing a specific Pro-Code could be resolved when there were different Pro-Code expressing cells together, 293T (human embryonic kidney cells), THP1 (human monocytic cells), 4T1 (mouse mammary cancer), and Jurkat (human T cells) cells were transduced with a pool of 18 Pro-Code vectors. The cells were transduced at a low multiplicity of infection (“MOI”) so that each cell was only transduced with a single Pro-Code vector. After 1 week, cells were harvested and stained with antibodies against dNGFR and all 10 of the linear epitopes. Each antibody was conjugated with a different metal, and samples were analyzed on a CyTOF mass cytometer (FIG. 1B). Mass spectometry permits detection of over 45 different metal-conjugated antibodies (Bendall et al., “Since-Cell Mass Spectrometry of Differential Immune and Drug Responses Across a Human Hematopoietic Continuum,” Science 332:687-696 (2011), which is hereby incorporated by reference in its entirety), and would thus enable detection of the Pro-Code epitopes along with more than 35 phenotypic markers. All 10 epitope tags were detected with a clear signal over background, and all of the epitope-positive cells were positive for NGFR (FIG. 1C).

To determine if cells expressing specific Pro-Codes could be resolved, NGFR⁺ cells were analyzed using a debarcoder algorithm (Fread et al., “An Unpdated Debarcoding Tool for Mass Cytometry with Cell Type-Specific and Cell Sample-Specific Stringency Adjustment,” Pacific Symp. Biocomput. 22:588-598 (2017), which is hereby incorporated by reference in its entirety). Eighteen distinct cell populations were detected (FIGS. 1D and 1E), with each population corresponding to a unique Pro-Code (i.e. positive for precisely 3 of the 10 epitopes). For example, one population of cells was positive for the E3, E4, and E5 epitopes, and negative for all other epitopes, indicating the cells expressed the E3-E4-E5 Pro-Code (FIG. 1F). The dimensional reduction algorithm viSNE (Amir et al., “viSNE Enables Visualization of High Dimensional Single-Cell Data and Reveals Phenotypic Heterogeneity of Leukemia,” Nat. Biotechnol. 31:545-552 (2013), which is hereby incorporated by reference in its entirety) was used to cluster the NGFR-positive cells based on their epitope tag expression. Once again, 18 distinct populations of cells were identified with each cluster being positive for only 3 epitopes, and thus corresponding precisely to a specific Pro-Code (FIGS. 1G and 1H). To determine if the number of epitopes per Pro-Code could be increased, 14 Pro-Codes with 4 epitopes per Pro-Code were generated. Each one was cloned into a lentiviral vector. 293T cells were transduced with the 14 vector pool at low MOI, and cells were analyzed by CyTOF. All 10 epitopes were detected and cells were positive for 4 epitopes. This enabled the identification of all 14 4-epitope Pro-Code populations (FIG. 1I).

Next, whether a more complex mixture of Pro-Codes could be resolved in cells was investigated. 120 different 3-epitope Pro-Code plasmids were pooled together in a roughly equimolar ratio and used to make a library of lentiviral vectors. 293T cells, as well as monocytic cells (THP1), leukemic T cells (Jurkat), and mammary carcinoma cells (4T1) were transduced with the 120 vector library at a low MOI. After 1 week, cells were stained with the 10 metal-conjugated antibodies, and analyzed by CyTOF. Unsupervised clustering by viSNE analysis resolved 120 distinct populations (FIGS. 1J-1M), with each population corresponding precisely to one Pro-Code vector (FIGS. 1N-1Q). The frequency of each population ranged from 0.1% to 3%, with the majority of Pro-Code populations (65%) being between 0.4-1.5% (FIG. 1R), which is close to the expected frequency of 0.83% if each of the 120 Pro-Codes was in equimolar concentration.

Using an expanded set of 14 epitopes, 364 3-epitope Pro-Code vectors were generated and introduced into 293T cells by low MOI transduction. Transfected cells were stained for dNGFR and all 14 epitopes, analyzed by CyTOF, and all 364 Pro-Code expressing populations were readily identified and clustered (FIGS. 1S-1U). Thus, with only 14 antibodies (i.e., 14 detection channels), 364 different vector expressing cell populations could be detected. These results demonstrate that combinations of linear epitopes can be used to generate protein barcodes that are detectable at the protein level and at single-cell resolution.

### Example 2—Pro-Codes can be Used In Vivo to Track Cancer Cell Growth

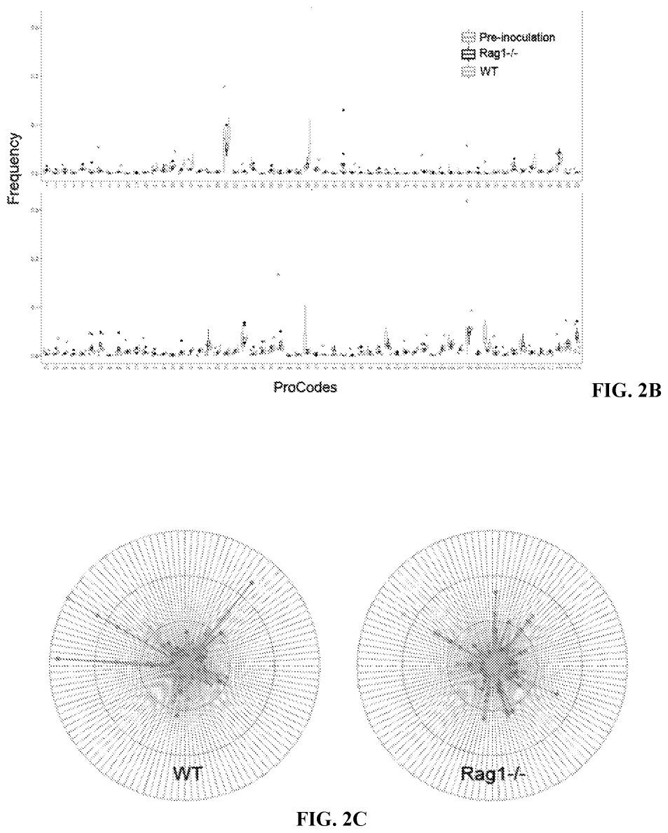

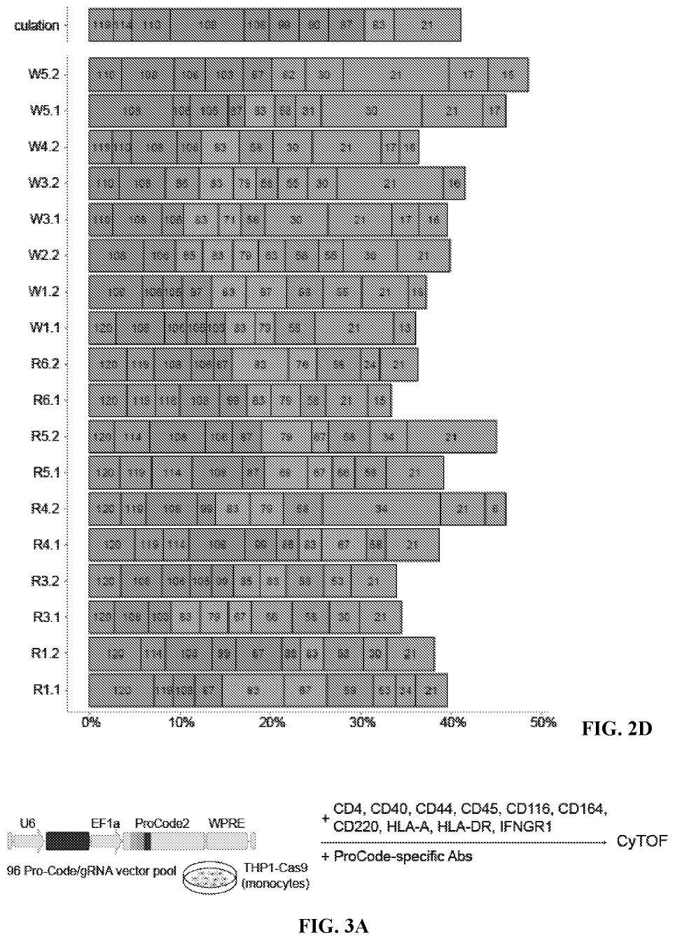

One important application of vector barcoding technology has been its use in cell clone and lineage tracing (Lu et al., “Tracking Single Hematopoietic Stem Cells In Vivo Using High-Throughput Sequencing in Conjunction with Viral Genetic Barcoding,” Nat. Biotechnol. 29:928-934 (2011), which is hereby incorporated by reference in its entirety). Fluorescent proteins have provided a powerful way to do this (Livet et al., “Transgenic Strategies for Combinatorial Expression of Fluorescent Proteins in the Nervous System,” Nature 450:56-62 (2007), which is hereby incorporated by reference in its entirety), but the number of populations that can be tracked is quite limited. DNA barcodes can tag an almost infinite number of cells, but only provide bulk resolution. The Pro-Codes of the present invention could potentially be used for clone tracking, but an important requirement is that they can be used in vivo. To address this, 4T1 mammary carcinoma cells were transduced with a pool of 120 Pro-Code vectors. A low MOI was used to achieve a single vector copy per cell. Cells were then sorted based on NGFR, as dNGFR serves not only as a Pro-Code scaffold, but also can be used as a selectable marker of transduced cells. The transduced cells were injected in to the right and left mammary gland of wildtype (WT) mice (n=5 mice, 2 tumors per mouse) (FIG. 2A). Since cells expressing non-self-proteins can be subject to immune clearance in immunocompetent animals, Rag1−/− immunodeficient mice were injected for comparison (n=6 mice, 2 tumors per mouse).

Mice were sacrificed 14 days after cell injection, and 18 different tumors were removed, and cultured for 3 days to enrich for the cancer cells. The cells were then stained for NGFR and each of the 10 Pro-Code epitopes. 118-120 Pro-Code expressing populations of cancer cells were identified in each tumor (FIG. 2B). While the proportion of each subpopulation varied for different Pro-Codes, this reflected a bias in the original population, as indicated by the comparison of each Pro-Code's frequency in the pre-inoculation cells compared to their frequency in the tumors. Importantly, there was no significant difference in the proportion of the vast majority of Pro-Code populations in WT or Rag1^{-/-} mice. This demonstrates that the Pro-Codes of the present invention are not differentially rejected, and thus can be used for in vivo experiments in wildtype and immune compromised mice.

The analysis of the composition of individual tumors revealed that, although each mouse was injected with the same pool of cells, the Pro-Code composition of each tumor was different (FIG. 2C). While most individual Pro-Codes were present in less than 1% of tumor cells, there was variability in the percent of each Pro-Code between tumors and mice. The proportion of the 10 most abundant Pro-Codes in each tumor is plotted in FIG. 2D. The same initial mix of 120 Pro-Code subpopulations developed into heterogenic tumors, in which 10 populations accounted for up to 50% of the total cell number. Overall, only 37 Pro-Code subpopulations were present at least once in the top 10 most represented populations in a tumor. Some Pro-Code populations were abundant in every tumor (e.g., Pro-Codes 108 and 21), but their proportion within each tumor varied greatly. For example, Pro-Code 21 was present in 3.5% of cells from one tumor, and 11.6% of another tumor. Other Pro-Code populations were only abundant in a single tumor, such as Pro-Code 6, which represented 2.3% of one tumor, but was one of the lowest represented populations in other tumors (FIG. 2B). These results support a model in which clonal growth was largely stochastic and not impacted by the Pro-Codes, and demonstrate that Pro-Codes can be used for cell tracking studies.

### Example 3—Pro-Codes Allow for High Dimensional Phenotyping of CRISPR Screens with Single Cell Resolution

One application of Pro-Code technology is the addition of protein-level phenotyping in genetic screens. It was hypothesized that a CRISPR gRNA can be paired with a specific Pro-Code, and this will enable cells expressing the gRNA to be detectable by CyTOF. To test this hypothesis, 96 CRISPR gRNAs targeting 54 different genes (1-3 guide RNAs per gene) were generated and paired with a different Pro-Code. Since packaging vector pools together can lead to varying degrees of barcode swapping (Hill et al., “On the Design of CRISPR-Based Single-Cell Molecular Screens,” Nat. Methods 15:271-274 (2018) and Sack et al., “Sources of Error in Mammalian Genetic Screens,” G3 6(9):2781-90 (2016), each of which is hereby incorporated by reference in its entirety), each vector was made individually and subsequently pooled in equimolar ratio to eliminate the possibility of template switching. THP1 human monocytes were engineered to stably express Cas9 (THP1-Cas9) and transduced with all 96 Pro-Code/CRISPR vectors together in a pool. Cells were cultured for 10 days and then stained with metal-conjugated antibodies specific for NGFR, all 10 linear epitopes, and the membrane-bound molecules CD4, CD40, CD44, CD45, CD116, CD164, CD220, HLA-A, HLA-DR, and IFNGR1, which were all targeted by CRISPR gRNAs included in the vector library (FIG. 3A). 500,000 cells were next analyzed by CyTOF. All 96 populations of Pro-Code expressing cells were resolved and clustered. This enabled examination of the expression of the surface proteins on each of the 96 Pro-Code/CRISPR populations with single cell resolution.

In each Pro-Code population in which one of the membrane-bound proteins was targeted, there was an increase in the percent of cells negative for the cognate protein (FIGS. 3B and 3C). For example, in cells expressing Pro-Code 3, which was linked to a gRNA targeting the CD4 gene, 85% of the cells were CD4 negative, whereas cells expressing Pro-Codes linked to gRNAs targeting unrelated genes were almost all CD4 positive (FIGS. 3B-3F). High efficiency protein loss was also observed for CD44, CD45, CD116, CD164, CD220, and IFNGR1. Though there was little evidence of knockout for some gRNAs, consistent with the known variability in CRISPR efficiency between gRNAs. These results demonstrate Pro-Codes can mark cells encoding a specific CRISPR gRNA, and show how this can be assessed by targeting KO of genes detectable by CyTOF. The data also demonstrate how Pro-Codes allow for simultaneous evaluation of the efficiency of multiple gRNAs.

In addition to directly measuring expression of the targeted gene, the high-dimensional phenotypic analysis of 10 proteins permitted by the Pro-Codes enabled examination of the potential impact of an edited gene on different biological markers (FIGS. 3B-3C). As an example, in cells expressing Pro-Code 24, which was linked to a gRNA targeting B2m, there was a significant loss of HLA-A. Whereas 96±3% of THP1 cells expressing other Pro-Code/CRISPRs were HLA-A positive, only 31% of cells expressing Pro-Code 24 (linked to B2m gRNA) were HLA-A positive, and 69% were HLA-A negative. This is expected based on B2m's role in stabilizing HLA (Zijlstra et al., “Beta 2-microglobulin deficient mice lack CD4-8+ cytolytic T cells,” Nature 344(6268):742-6 (1990), which is hereby incorporated by reference in its entirety). These results demonstrate how Pro-Codes can be used to enable protein-level phenotyping in pooled CRISPR screens.

The library pool used above was made with vectors packaged individually and pooled subsequently to prevent the possibility of barcode swapping. Recently it was reported that swapping can also be reduced by co-packaging libraries with a low homology transfer vector (Adamson et al., “Approaches to Maximize sgRNA-Barcode Coupling in Perturb-Seq Screens,” BioRxiv 298349 (2018) and Feldman et al., “Lentiviral Co-Packaging Mitigates the Effects of Intermolecular Recombination and Multiple Integrations in Pooled Genetic Screens,” BioRxiv 262121 (2018), each of which is hereby incorporated by reference in its entirety). To determine if this would be compatible with the Pro-Codes, a 96 Pro-Code/CRISPR library was produced as a pool and spiked in a plasmid encoding a lentivirus expressing GFP during vector packaging. THP1-Cas9 cells were transduced with the 96 Pro-Code/CRISPR library at low MOI. Cells were stained for NGFR, the Pro-Code epitopes, and all 10 membrane-bound molecules, as above. Cells were also stained for GFP to distinguish cells transduced with the GFP encoding lentivirus in the pool and analyzed cells by CyTOF. Similar to the library made with individually packaged vectors, all 96 Pro-Code populations could be resolved, and loss of a specific protein on a high percent of cells expressing a Pro-Code linked to a gRNA targeting the cognate gene was observed (FIG. 3E). The frequency of cells negative for the targeted protein was ˜90% similar between the libraries generated with vectors produced individually or as a pool with the low homology vector. These results indicate Pro-Code/CRISPR libraries can be produced as a pool and function at high efficiency, and further support the ability of Pro-Codes to facilitate high-dimensional (i.e., 10 protein) phenotypic screens.

### Example 4—Pro-Codes Enable Interrogation of Signaling Pathways in Reverse Genetic Screens

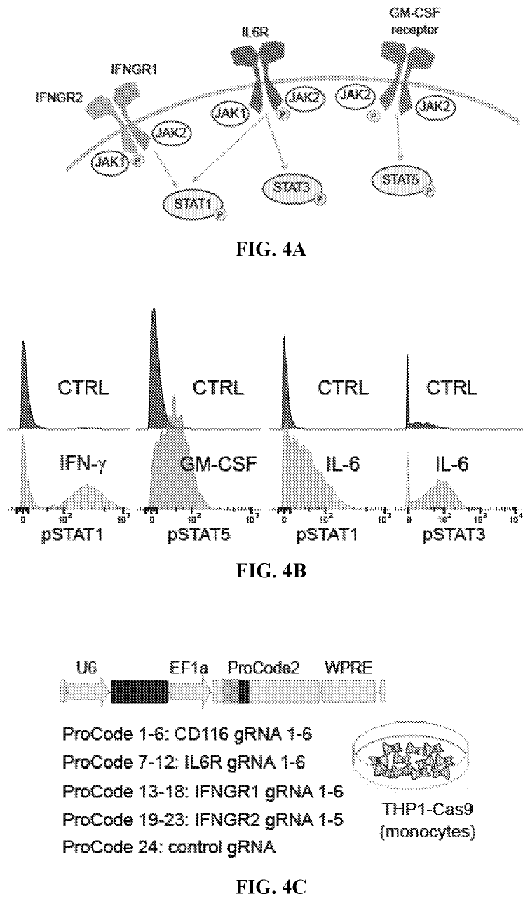

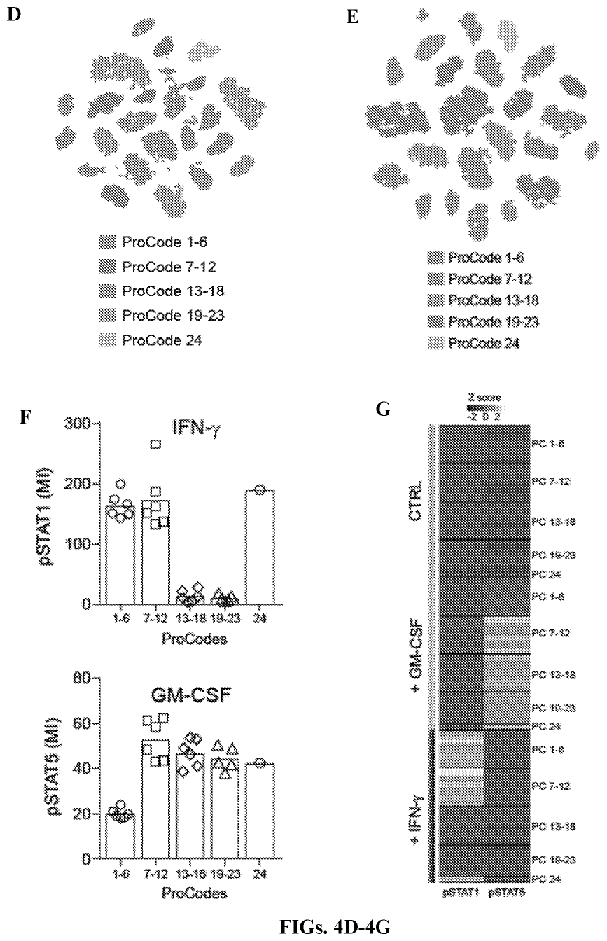

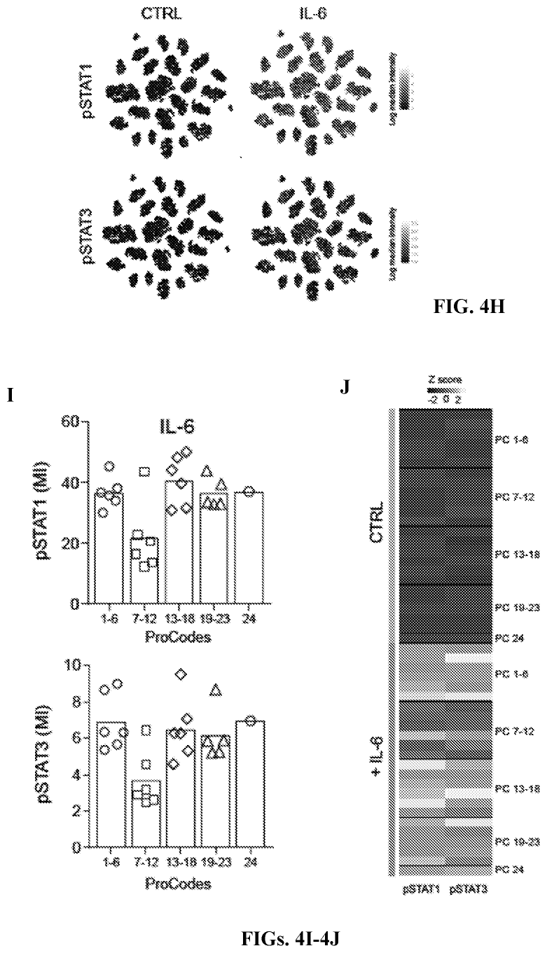

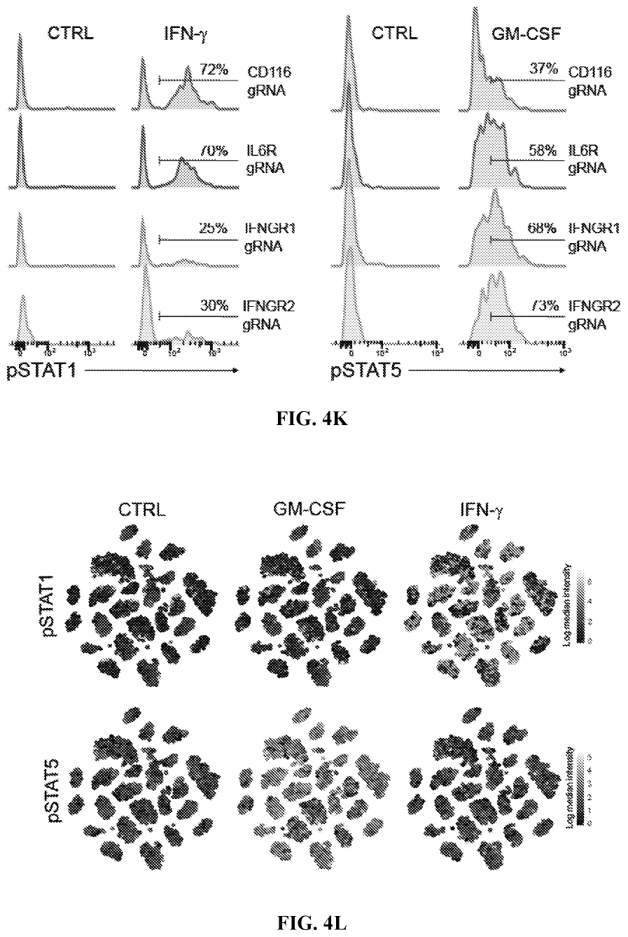

Intracellular signaling plays an essential role in numerous cellular processes. The activation and de-activation of specific proteins in signaling pathways is a post-translational event, and is thus optimally studied at the protein level. This makes it challenging to directly assess signaling alterations with current screening approaches. Whether Pro-Code technology would facilitate a genetic screen of signal transducer and activator of transcription (“STAT”) signaling was next evaluated. STAT proteins function downstream of cytokine receptors was next evaluated. When different cytokines engage their cognate receptors, specific STAT proteins are phosphorylated, and transmit the cytokine signal (O'Shea et al., “The JAK-STAT Pathway: Impact on Human Disease and Therapeutic Intervention,” Annu Rev Med. 66:311-28 (2015), which is hereby incorporated by reference in its entirety). IFNγ engagement of the IFNγ receptor (comprised of IFNGR1 and IFNGR2 subunits) triggers phosphorylation of STAT1 (pSTAT1), IL-6 engagement of the IL-6 receptor (IL6R) triggers phosphorylation of STAT1 and STAT3 (pSTAT3), and GM-CSF engagement of the GM-CSF receptor (CD116) triggers phosphorylation of STAT5 (pSTAT5) (FIG. 4A). This was assessed in culture by treating THP1 monocytes with IFNγ, GM-CSF, or IL-6, and analyzing pSTAT1, pSTAT3, and pSTAT5 by CyTOF. As expected, IFNγ led to increased pSTAT1, GM-CSF led to increased pSTAT5, and IL-6 led to increased pSTAT1 and pSTAT3 (FIG. 4B).

A library of 24 different lentiviral vectors, each encoding a different Pro-Code and gRNA (FIG. 4C) was constructed. The gRNAs were designed to target the IFNGR1, IFNGR2, IL6R, and CD116 genes. 5-6 gRNAs were generated per gene, as well as one control gRNA targeting an irrelevant gene. Each guide RNA was cloned with a different Pro-Code. THP1-Cas9 cells were transduced with the pool of Pro-Code/CRISPR vectors. After 1 week, cells were stimulated with IFNγ, GM-CSF, IL-6, or PBS. After 15 minutes the cells were fixed, stained with metal-conjugated antibodies specific for the Pro-Code epitopes as well as pSTAT1, pSTAT3, and pSTAT5, and analyzed by CyTOF. All 24 Pro-Code populations, corresponding to 24 different gRNA expressing populations, were resolved and uniquely clustered (FIGS. 4D and 4E).

The expression of pSTAT1, pSTAT3, and pSTAT5 in each Pro-Code population was examined. In all cases, evidence of a decrease in phospho-signaling was observed in cells expressing a Pro-Code linked to a CRISPR gRNA targeting the cognate receptor (FIGS. 4F-4J). Looking at the mean change in signaling, there was a 15-fold decrease in pSTAT1 levels in cells expressing Pro-Codes linked to gRNAs targeting IFNGR1 and IFNGR2 (FIGS. 4F-4G). Whereas in cells expressing the same Pro-Code/CRISPRs, pSTAT5, and pSTAT1 and pSTAT3 levels were normal in response to GM-CSF and IL-6. This indicated the IFNGR1 and IFNGR2 gRNAs only impaired pSTAT1 signaling in response to IFNγ. Similarly, in cells encoding the Pro-Codes linked to gRNAs targeting GM-CSF there was a 3-fold reduction in pSTAT5 levels in response to GM-CSF, and in cells carrying gRNAs targeting IL6R there was a 2-fold reduction in both pSTAT1 and pSTAT3 levels in response to IL-6 (FIGS. 4I-4J).

The ability to analyze cells at single cell resolution enabled investigation of the heterogeneity in each Pro-Code/CRISPR population of cells. When cells were treated with IFNγ, 70% of the cells in the Pro-Code clusters linked to gRNAs targeting CD116 and IL6R had increased pSTAT1, whereas in the Pro-Code clusters linked to gRNAs targeting IFNGR1 and IFNGR2, only ˜25% of the cells had increased pSTAT1 (FIGS. 4K-4L). When cells were treated with GM-CSF, 60-70% of the cells in the clusters encoding gRNAs targeting IL6R, IFNGR1, and IFNGR2 upregulated pSTAT5, but only 30-40% of the cells in the Pro-Code clusters encoding CD116 gRNAs upregulated pSTAT5 (FIGS. 4K-4L).

Looking at the viSNE clusters, in which each dot is representative of a single cell, there were cells positive and negative for pSTAT (FIG. 4L). Thus, while the bulk analysis indicated a major reduction in pSTAT signaling downstream of the receptor targeted by a specific CRISPR, single cell analysis indicated that there was significant heterogeneity between cells even within the same Pro-Code cluster. This heterogeneity reflects biological differences between cells in their response to cytokine stimulation, but also reveals cell-to-cell heterogeneity in CRISPR-mediated knockout, as observed in the studies above measuring the protein levels of the gene targeted by specific CRISPRs. The editing efficiency of CRISPR is variable (Dang et al., “Optimizing sgRNA Structure to Improve CRISPR-Cas9 Knockout Efficiency,” Genome Biol. 16:280 (2015) and Yuen et al., “CRISPR/Cas9-Mediated Gene Knockout is Insensitive to Target Copy Number but is Dependent on Guide RNA Potency and Cas9/sgRNA Threshold Expression Level,” Nucleic Acids Res. 45:12039-12053 (2017), each of which is hereby incorporated by reference in its entirety), and this highlights the important utility of single cell analysis in CRISPR screens. Together, these results demonstrate Pro-Codes enable direct single cell phenotypic analysis of signaling pathways in CRISPR screens, which is not feasible with DNA or RNA level analysis.

### Example 5—Pro-Code/CRISPR Screen Reveals Mechanisms of Cancer Resistance to Antigen-Specific Cytotoxic T Cells

Cancer cells acquire mutations which generate neo-antigens that are loaded on to MHC class I, and make the cancer cells targets for CD8+ T cell killing (Schumacher et al., “Neoantigens Encoded in the Cancer Genome,” Curr. Opin. Immunol. 41:98-103 (2016), which is hereby incorporated by reference in its entirety). However, cancer cells can alter their gene expression programs to resist being killed by the T-cells. Though some of the genes important for cancer cell sensitivity and resistance to immune editing have been identified, the potential contributions of many genes still need to be interrogated. Recently, several studies have used pooled CRISPR screens, using DNA barcodes for deconvolution, to identify novel sensitivity and resistance genes (Konermann et al., “Genome-Scale Transcriptional Activation by an Engineered CRISPR-Cas9 Complex,” Nature 517:583-588 (2014); Pan et al., “A Major Chromatin Regulator Determines Resistance of Tumor Cells to T Cell—Mediated Killing,” Science 359(6377):770-775 (2018); and Patel et al., “Identification of Essential Genes for Cancer Immunotherapy,” Nature 548:537-542 (2017), each of which is hereby incorporated by reference in its entirety). It was investigated whether Pro-Code technology could be used to aid in the identification of genes conferring cancer cell sensitivity or resistance to T-cell immunity.

A library of 56 CRISPR gRNAs targeting 14 different genes (3 to 4 gRNAs/gene) was generated and each CRISPR was paired with a unique Pro-Code to form a pool of 56 Pro-Code/CRISPR vectors (including 4 scrambled gRNAs) (FIG. 5A). 14 genes known to contain regulators of immunity (such as B2m) and several genes with no known role (such as Cldn4) were selected. The 4T1 mammary carcinoma line was used as a model of breast cancer. In previous screens, antigen-specific T-cells targeting model tumor associated antigen (“TAA”), such as OVA, gp100, and NY-ESO-1 were utilized (Manguso et al., “In vivo CRISPR Screening Identifies Ptpn2 as a Cancer Immunotherapy Target,” Nature 547:413-418 (2017); Pan et al., “A Major Chromatin Regulator Determines Resistance of Tumor Cells to T Cell—Mediated Killing,” Science 359(6377):770-775 (2018); and Patel et al., “Identification of Essential Genes for Cancer Immunotherapy,” Nature 548:537-542 (2017), each of which is hereby incorporated by reference in its entirety). A caveat of these antigens is that they are not readily detected in cells. To overcome this limitation, eGFP death inducing (Jedi) T-cells, which express a T-cell receptor that recognizes the immunodominant epitope of GFP loaded in the H-2Kd allele of MHC class I (Agudo et al., “GFP-Specific CD8 T Cells Enable Targeted Cell Depletion and Visualization of T-Cell Interactions,” Nat. Biotechnol. 33:1287-1292 (2015), which is hereby incorporated by reference in its entirety), were utilized. Jedi T-cells enable GFP to be used as a model antigen that can be easily detected. 4T1 cells were engineered to express either GFP (4T1-GFP) or near-infrared fluorescent protein 670 (4T1-RFP) alone, or with Cas9 (4T1-Cas9-GFP and 4T1-Cas9-RFP). When the cells were co-cultured with activated CD8⁺ Jedi T-cells there was selective killing of the GFP⁺ cells, which could be quantified by flow cytometry (FIGS. 5B-5C). Thus, this system enables precise analysis of antigen-specific T-cell killing. The inclusion of RFP⁺ cells serves as an internal control of non-TAA expressing cells, and enables distinction between the effects of a specific knockout on cell fitness versus T-cell sensitivity.

Each group of 4T1 cells (4T1-GFP, 4T1-RFP, 4T1-Cas9-GFP, and 4T1-Cas9-RFP) was transduced with the library of Pro-Code/CRISPR vectors. After 10 days, 4T1-Cas9-GFP and 4T1-Cas9-RFP (or 4T1-GFP and 4T1-RFP) cells were mixed in a 1:1 ratio, and co-cultured with activated CD8⁺ Jedi T-cells (FIG. 5A). Bulk comparison of the frequency of GFP⁺ and RFP⁺ cells indicated that the GFP⁺ cells were almost completely eliminated in the Cas9 null cultures with the activated Jedi T-cells (FIG. 5B). In contrast, a large fraction of 4T1-Cas9-GFP cells survived (8-12% of the culture), despite their expression of the antigenic target of the T-cells (FIG. 5C). These results suggest that gene editing results in resistant cancer cells, and since the fraction of resistant cells did not change at the highest ratio of T-cells, this further suggests that resistance was robust.

To determine which genes may be involved in 4T1 resistance or sensitivity to T-cell killing, we stained the cells with metal-conjugated antibodies for the Pro-Code epitopes, as well as GFP, CD45 and MHC class I (H-2Kd), and analyzed by CyTOF. Each of the 56 Pro-Code expressing populations were detected, and resolved by viSNE (FIGS. 5D-5E). There were no changes in the relative frequency of specific Pro-Code expressing populations in 4T1-RFP cells, with or without Cas9, in the presence or absence of Jedi T-cells (FIGS. 5D-5E). Examination of the Pro-Code markers in the surviving 4T1-Cas9-GFP population revealed enrichment of cells expressing Pro-Codes linked to gRNAs targeting Ifngr2 and B2m (FIGS. 5E-5H). Approximately 39% of the surviving cancer cells carried an Ifngr2 CRISPR (FIG. 5G). A similar result was seen when experiments were performed with individual CRISPRs targeting only B2m or Ifngr2 (FIG. 5I). These findings are consistent with emerging clinical data correlating resistance to checkpoint inhibitors with mutations in the B2m and IFNγ pathways (Gao et al., “Loss of IFN-γ Pathway Genes in Tumor Cells as a Mechanism of Resistance to Anti-CTLA-4 Therapy,” Cell 167(2):397-404.e9 (2016) and Zaretsky et al., “Mutations Associated with Acquired Resistance to PD-1 Blockade in Melanoma,” N. Engl. J. Med. 375:819-829 (2016), each of which is hereby incorporated by reference in its entirety), and with recent genome-wide CRISPR screening data (Patel et al., “Identification of Essential Genes for Cancer Immunotherapy,” Nature 548:537-542 (2017), which is hereby incorporated by reference in its entirety).

Because Pro-Code technology allows analysis at the protein level with single cell resolution, the expression of both the TAA (GFP) and MHC class I could be examined on each cell. As expected, lower MHC class I was detected on cells encoding the B2m gRNAs (FIG. 5J). In cells encoding Ifngr2 CRISPRs, there were normal levels of MHC class I expression in steady-state, but the expression of MHC class I on these cells did not increase in the Jedi co-cultures, as it did in cells carrying unrelated CRISPRs. This suggests that one of the mechanisms by which the Ifngr2 CRISPR cells resisted T-cell killing may be due to diminished upregulation of MHC class I.

In addition to the B2m and Ifngr2 CRISPR populations, there were residual cells remaining in each Pro-Code/CRISPR population after Jedi co-culture (FIGS. 5D-5E). This implies that there was resistance independent of the specific gene perturbation. Because GFP and MEW class I was measured, these factors could be examined as a potential mechanism. Interestingly, a common feature of the cells that remained across most Pro-Code populations was decreased GFP or MEW class I expression (FIGS. 5K-5M). Looking at the single cell level, many GFP^{low} and H-2Kd^{low} (MEW class I) cells were found to be mutually exclusive, indicating antigen loss and downregulation of the presentation pathway often occurred as divergent pathways of resistance (FIGS. 5L and 5N). Since it is possible some of the H-2Kd^{low} cells in the different Pro-Code populations could have resulted from a B2m gRNA swapping in to another Pro-Code vector, the same experiment as above was performed with individual Pro-Code/CRISPR vectors encoding a scrambled gRNA. As observed with the pool of vectors, in cultures containing activated Jedi T-cells, there emerged populations of 4T1-GFP that had downregulated H-2Kd or GFP and escaped T-cell killing (FIG. 5O), supporting the notion that this mechanism can arise spontaneously.

### Example 6—the IFNγ Inducible Genes Psmb8 and Rtp4 Influence Susceptibility to Antigen-Dependent T Cell Killing

Though the cells carrying the Ifngr2 CRISPR did not upregulate MHC class I in response to IFNγ, the cells still expressed high levels of MEW class I (FIG. 5J). Indeed, the levels of MI-IC class I were comparable to the activated Jedi T-cells. Since there are many facets of the IFNγ pathway, other components of the pathway were investigated to determine what may influence cancer resistance to T-cell killing. Genes associated with the IFNγ pathway, as well as several genes with no reported associations (Socs1-7, Ptpn1, Ptpn2, Rtp4, Rab5b, Stip1, Supt16, and Psmb8) were selected. 2-4 gRNAs were designed per gene. Each gRNA was cloned into a Pro-Code construct. A pool of 56 Pro-Code/CRISPR lentiviral vectors were generated and used to transduce 4T1-GFP-Cas9 and 4T1-Cas9-mCherry cells. The transduced populations were mixed in a 1:1 ratio and co-cultured with or without activated Jedi T-cells. On day 3, cells were collected and stained with metal-conjugated antibodies for the Pro-Code epitopes, as well as GFP, mCherry, CD45, MHC class I (H-2Kd), and PD-L1 for analysis by by CyTOF.

Bulk comparison of GFP⁺ and mCherry⁺ cells found that a fraction of GFP⁺ cells survived, indicating resistant cancer cells had emerged (FIG. 6A). Cells exposed to activated Jedi T-cells upregulated both MHC class I and PD-L1 (FIGS. 6B-6C). Interestingly, when PD-L1 expression was investigated on specific Pro-Code populations, all 3 populations expressing a Pro-Code linked to a gRNA targeting Socs1 had increased upregulation of PD-L1 (FIG. 6D). This was specific to PD-L1 because the same population of cells had similar levels of MHC class I to other Pro-Code/CRISPR populations (FIG. 6E). These results implicate Socs1 as a negative regulator of PD-L1.

Next, changes in the frequency of specific Pro-Code populations were examined within the GFP and mCherry cell fractions (FIG. 6F). To allow for comparison across 4 independent experiments, these changes were expressed as a function of killing of the GFP⁺ cells. Examination of the Pro-Code markers revealed that cells expressing Pro-Codes linked to gRNAs targeting Psmb8 and Rtp4 were enriched in the surviving 4T1-Cas9-GFP populations. The frequency of 4T1-Cas9-mCherry cells expressing Psmb8 and Rtp4 gRNAs did not significantly change, indicating enrichment was dependent on antigen-specific T-cell killing.

To validate these findings, 4T1-Cas9-GFP cells were transduced with either gRNAs targeting Psmb8 or Rtp4, or a scramble gRNA, mixed in 1:1 ratio with 4T1-Cas9-mCherry cells and co-cultured with activated CD8⁺ Jedi T-cells. In support of the screen results, increased resistance of cells encoding the Psmb8 and Rtp4 CRISPR was observed compared to the scramble control (FIGS. 6G-6H). Whereas <0.1% of control 4T1-GFP cells remained in the Jedi co-cultures, ˜4% of the Rtp4 CRISPR and 10% of the Psmb8 CRISPR 4T1-GFP cells remained.

Though not all transduced cells were resistant, this was expected because not all of the cells will be a complete knockout for either Rtp4 or Psmb8, due to the variability in CRISPR efficiency. Thus, the percent of cells remaining reflects resistance to antigen-specific T-cell killing, but does not provide an indication of the robustness of resistance. To address this, 4T1-Cas9-GFP cells expressing the Rtp4 or Psmb8 gRNA were co-cultured with activated Jedi T-cells, and the GFP⁺ resistant cells were expanded (FIG. 6I). The cells were mixed with 4T1-Cas9-mCherry cells in a 1:1 ratio and re-cultured with activated Jedi T-cells. Strikingly, the Psmb8 and Rtp4 KO cells were almost completely resistant to T-cell killing (FIG. 6J). Western blot confirmed Psmb8 protein was absent in the expanded Psmb8 CRISPR 4T1 cells (FIG. 6K). Because there was not a satisfactory antibody for Rtp4 protein detection, Sanger and qPCR was used to confirm the Rtp4 gene had been extensively mutated and was no longer expressed in the Rtp4 KO cells (FIGS. 6L-6M). Together, these results indicate that Psmb8 and Rtp4 have a non-redundant role in mediating sensitivity of tumor cells to antigen-dependent T-cell killing.

### Discussion of Examples 1-6

Examples 1-6 describe a new technology for cell and vector barcoding, which uses combinations of linear epitopes to create a higher multiple of protein barcodes. These examples demonstrate the generation and resolution of 364 unique Pro-Codes using 14 epitope and antibody pairs for construction and detection. While this is far fewer barcodes than achieved with DNA, it is an order of magnitude greater than what currently exists with protein reporters. Moreover, thousands of new Pro-Codes can be created simply by introducing additional epitopes and epitope positions. Although generating genome-wide Pro-Code/CRISPR libraries cannot be done at the relative ease with which DNA barcoded libraries can be made using arrayed synthesis and shotgun cloning, Pro-Code technology's application to reverse genetics will likely be primarily for more focused screens, concentrating on specific pathways or gene classes, and targeting 100-500 genes. As more linear epitopes are validated, it will also be possible to create CRISPR libraries with non-overlapping Pro-Codes, and use them together to perform complex screens to identify cooperating or redundant genes in a relatively unbiased manner.

An important advance provided by the Pro-Code technology is the ability to perform high-dimensional phenotyping of multiple proteins in pooled genetic screens, as demonstrated above. This is not feasible with DNA as the barcode, as the screen readout would be limited to measuring changes in barcode frequency, and inferring phenotype based on the selective pressure applied. By being able to mark hundreds of different CRISPR-expressing populations and measure many protein markers, Pro-Code technology expands the types of pooled genetic screens that can be performed, and will help facilitate the annotation of gene functions.

A key feature of Pro-Codes technology is that it enables screens to be performed with single cell resolution. For CRISPR screens, single cell analysis is particularly relevant because the efficiency of CRISPR knockout is highly variable; some cells may be complete KO, while other cells have only a partial KO or remain wildtype. This was evident from the phenotypic analysis in which only a fraction of cells expressing a particular Pro-Code/CRISPR were negative for the cognate protein described above (FIGS. 3A-3C). As DNA barcode deconvolution is generally performed on bulk cells, this means cells with complete, partial, or no KO are lumped together in the analysis. Even if there is an effect of complete KO, the magnitude is diluted by the wildtype cells. With Pro-Code technology, every cell expressing a CRISPR can be analyzed individually. Even when the targeted gene itself is not analyzed, the phenotypic differences can be seen between individual cells receiving the same CRISPR, as observed in the Pro-Code/CRISPR analysis of phospho-STAT signaling (FIG. 4L), as well as PD-L1 (FIG. 6D). Moreover, as opposed to DNA barcodes in which the percent of each vector is presumed from sequence frequency, with Pro-Code technology, the frequency of each CRISPR-carrying cell within a population is directly determined. This enables precise consideration of the number of cells sampled in each population and informs analysis.

Several groups have incorporated scRNA-seq into pooled screens to obtain more comprehensive phenotyping than had previously been possible with pooled genetic screens, and to achieve single cell resolution (Adamson et al., “A Multiplexed Single-Cell CRISPR Screening Platform Enables Systematic Dissection of the Unfolded Protein Response,” Cell 167:1867-1882 (2016); Datlinger et al., “Pooled CRISPR Screening with Single-Cell Transcriptome Readout,” Nat. Methods 14:297-301 (2017); Dixit et al., “Perturb-Seq: Dissecting Molecular Circuits with Scalable Single-Cell RNA Profiling of Pooled Genetic Screens,” Cell 167:1853-1866 (2016); and Jaitin et al., “Dissecting Immune Circuits by Linking CRISPR-Pooled Screens with Single-Cell RNA-Seq,” Cell 167:1883-1896 (2016), each of which is hereby incorporated by reference in its entirety). This provides a powerful advance to pooled screening approaches. However, the cell throughput of scRNA-seq is still relatively limited compared to what can be readily achieved with CyTOF (thousands versus millions), and the efficiency of transcript capture makes it challenging to quantitatively compare gene expression on a per cell basis without imputing gene levels. As gene editing does not necessarily affect the level of a target transcript, it is also difficult to directly determine if a particular gene has been functionally knocked out by scRNA-seq. Pro-Code technology makes it possible to analyze millions of single cells with precise quantification of protein levels. Though the number of genes that can be analyzed by CyTOF is fewer than scRNA-seq, it should be feasible to expand the phenotyping space by using oligonucleotide-labeled antibodies to detect the Pro-Codes and other proteins, and to deconvolute with single cell sequencing, as has recently been described (Peterson et al., “Multiplexed Quantification of Proteins and Transcripts in Single Cells,” Nat. Biotechnol. 35:936-939 (2017) and Stoeckius et al., “Simultaneous Epitope and Transcriptome Measurement in Single Cells,” Nat. Methods 14:865-868, each of which is hereby incorporated by reference in its entirety). As protein detection appears to be more consistent than RNA capture with single cell sequencing approaches, oligo-labeled antibody detection of Pro-Codes could help alleviate the issue of barcode dropout in scRNA-seq based CRISPR screens.

As noted, barcode swapping can occur in retroviral vector libraries packaged as pools, and the degree of swapping can range from 6% to 50%, depending on the distance between the barcode and effector molecule (i.e., the gRNA, shRNA, or cDNA) (Hill et al., “On the Design of CRISPR-Based Single-Cell Molecular Screens,” Nat. Methods 15:271-274 (2018) and Sack et al., “Sources of Error in Mammalian Genetic Screens,” Genes, Genomes, Genetics 6:2781-2790 (2016), each of which is hereby incorporated by reference in its entirety). Swapping occurs when two different vector genomes are packaged in the same virion, and there is template switching during reverse transcription. Fortunately, swapping can be prevented by packaging each vector individually, and pooling them subsequently, as done by Adamson et al., “A Multiplexed Single-Cell CRISPR Screening Platform Enables Systematic Dissection of the Unfolded Protein Response,” Cell 167:1867-1882 (2016) (which is hereby incorporated by reference in its entirety) and described above. Another approach to reduce the possibility of barcode swapping, which still enables the vector to be made as a pool, is to spike in a ‘decoy’ plasmid during vector production. This approach has been used in the HIV field to study template switching (King et al., “Pseudodiploid Genome Organization Aids Full-Length Human Immunodeficiency Virus Type 1 DNA Synthesis,” J. Virol. 82:2376-2384 (2008), which is hereby incorporated by reference in its entirety), and was recently described for making CRISPR lentiviral pools (Adamson et al., “Approaches to Maximize sgRNA-Barcode Coupling in Perturb-seq Screens,” BioRxiv 298349 (2018) and Feldman et al., “Lentiviral Co-Packaging Mitigates the Effects of Intermolecular Recombination and Multiple Integrations in Pooled Genetic Screens,” BioRxiv 262121 (2018), each of which is hereby incorporated by reference in its entirety). In this approach, a plasmid is spiked in to the packaging plasmid mixture in excess of the library plasmids. The plasmid encodes a vector genome that can be packaged in to the virion particle, but does not contain extensive homology to the library genome. In this way, there will be a high probability that vector particles will contain only a single genome encoding a CRISPR and barcode sequence. The other genome in the particle will not result in productive template switching. That this approach could also be used to make Pro-Code/CRISPR library as a pool and results in similar knockout efficiency as libraries made with individually packaged vectors was also confirmed.

In this study, CyTOF was utilized for Pro-Code detection because it enabled concurrent detection of additional proteins. It should be possible to detect Pro-Codes by flow cytometry, and this could be used to sort particular Pro-Code-expressing populations for expansion and further study. There is also the potential to utilize Pro-Code technology with advanced histological techniques, and add spatial mapping to CRISPR screens. There are now at least two platforms that enable high-dimensional tissue imaging with metal-conjugated antibodies, allowing over 40 parameters to be simultaneously detected in a single section, with subcellular resolution and in a highly quantitative manner (Angelo et al., “Multiplexed Ion Beam Imaging of Human Breast Tumors,” Nat. Med. 20:436-442 (2014) and Giesen et al., “Highly Multiplexed Imaging of Tumor Tissues with Subcellular Resolution by Mass Cytometry,” Nat. Methods 11(4):417-22 (2014), each of which is hereby incorporated by reference in its entirety). This enables each of the Pro-Code epitopes to be detected, and thus hundreds to thousands of barcoded cells to be resolved in a tissue section, along with more than 30 different protein markers of cell identity and function. In addition to adding a new dimension to genetic screens that is not currently feasible with DNA barcodes or scRNA-seq, mass-spectrometry based tissue analysis of the Pro-Codes could provide new possibilities for studying tumor clonality and lineage tracing in situ.

As described above, Pro-Code technology was used to carry out CRISPR screens aimed at identifying genes that influence sensitivity to antigen-specific T-cell killing. The screens were primarily intended as proof-of-principle studies, and were thus relatively small and included genes with established importance, such as B2m and Ifngr2. The IFNγ pathway has been implicated as a key component in the clinical response to checkpoint inhibitors (Minn et al., “Combination Cancer Therapies with Immune Checkpoint Blockade: Convergence on Interferon Signaling,” Cell 165:272-275 (2016), which is hereby incorporated by reference in its entirety). Mutations in IFNGR1 and JAK, a component of the IFNγ signaling pathway, have been found in patients presenting resistance to checkpoint inhibitors (Gao et al., “Loss of IFN-γ Pathway Genes in Tumor Cells as a Mechanism of Resistance to Anti-CTLA-4 Therapy,” Cell 167(2):397-404.e9 (2016) and Zaretsky et al., “Mutations Associated with Acquired Resistance to PD-1 Blockade in Melanoma,” N. Engl. J. Med. 375:819-829 (2016), each of which is hereby incorporated by reference in its entirety). However, the mechanisms that make IFNγ signaling essential to immune editing are not well established. Our studies found that knockout of two IFNγ inducible genes, Psmb8 and Rtp4, resulted in resistance to antigen-specific T-cell killing. Psmb8 (also known as Lmp7) is a component of the immunoproteasome, which functions in generating peptides for MHC class I (Basler et al., “The Immunoproteasome in Antigen Processing and Other Immunological Functions,” Curr. Opin. Immunol. 25:74-80 (2013), which is hereby incorporated by reference in its entirety), and its expression has been found to positively correlate with tumor-infiltrating lymphocyte abundance in breast cancer (Lee et al., “Expression of Immunoproteasome Subunit LMP7 in Breast Cancer and Its Association with Immune-Related Markers,” Cancer Res. Treat. (2018), which is hereby incorporated by reference in its entirety). Rtp4 (Receptor transporter protein 4) is a chaperone protein involved in the folding of G protein coupled receptors (“GPCR”) (Decaillot et al., “Cell Surface Targeting of mu-delta Opioid Receptor Heterodimers by RTP4,” Proc. Natl. Acad. Sci. 105:16045-16050 (2008), which is hereby incorporated by reference in its entirety). The only defined protein targets of Rtp4 are opioid receptors (Decaillot et al., “Cell Surface Targeting of mu-delta Opioid Receptor Heterodimers by RTP4,” Proc. Natl. Acad. Sci. 105:16045-16050 (2008), which is hereby incorporated by reference in its entirety), and, despite being an interferon stimulated gene, almost nothing is known about the role of Rtp4 in immunity. Future studies will be needed to understand how Rtp4 influences cell sensitivity to T cell killing, and to determine its relevance to immune editing of patient tumors. As Rtp4 is part of a family of chaperones proteins (Saito et al., “RTP Family Members Induce Functional Expression of Mammalian Odorant Receptors,” Cell 119:679-691 (2004), which is hereby incorporated by reference in its entirety), it will also be valuable to know if other RTPs have a role in sensitivity or resistance to immunity.

The importance of analyzing phenotypic markers in the screen was highlighted by the discovery that many resistant cells had lower levels of MHC class I or the target antigen, GFP. This would not be picked up in screens using DNA barcodes and could lead to artifactual findings as gRNA encoding vectors become passengers to naturally emerged resistance. While it is not surprising that loss of antigen or MHC class I would enable cancer cells to resist killing by antigen-specific T-cells, the results described above found that downregulation, and not just loss, of either factor also provided a survival advantage to the cancer cells. This may be underappreciated as a mechanism of cancer resistance to cytotoxic T-cell clearance, as subtle reductions in the expression of neo-antigens on individual cancer cells has not been widely examined in tumors owing to the challenge of making these measurements. Though the experimental system used is highly reductionist compared to the complexity of a tumor, it is also a very sensitive model; comprised of a high ratio of antigen-specific T-cells to antigen-bearing cancer cells. Thus, it may even underestimate the sensitivity of immune editing to reductions in antigen levels. Understanding the quantitative relationship between presentation components, neoantigen levels, and the immunotherapy response at high resolution in patient's tumors is needed, especially as neo-antigen prediction and neo-antigen vaccines (Ott et al., “An Immunogenic Personal Neoantigen Vaccine for Patients with Melanoma,” Nature 547:217-221 (2017), which is hereby incorporated by reference in its entirety) become more widely used in cancer immunotherapy.

### Example 7—GFP can Serve as an Alternative Pro-Code Scaffold

Whether GFP could be used as a scaffold for the Pro-Codes was next evaluated. A combination of 3 epitopes was cloned into a GFP transgene in a LV (FIG. 7A). 293T cells were transduced and cells were analyzed for the expression of GFP and the 3 epitopes using metal-conjugated antibodies. Because GFP is a cytoplasmic protein, staining was performed with a protocol optimized for intracellular detection. The cells were analyzed by CyTOF. GFP was detected in 49% of cells and, importantly, every cell that expressed GFP also expressed each of the 3 epitopes (FIG. 7B). This indicates that GFP can be used as a scaffold protein for the Pro-Codes.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A fusion protein comprising:
a scaffold protein and
a series of two or more distinct epitopes, wherein the distinct epitopes are recognized by distinct antibodies, and wherein the series of epitopes forms a detectable protein tag.

2. The fusion protein of claim 1, wherein each of the two or more epitopes is selected from HA, FLAG, VSVg, V5, AU1, AU5, Strep I, E, E2, Strep II, HSV, protein C tag, S-tag, OLLAS, HAT, and Tag-100-tag.

3. The fusion protein of claim 1 further comprising:
amino acid spacer sequences separating each of the two or more epitopes from each other.

4. The fusion protein of claim 1, wherein the scaffold protein is a cell surface protein.

5. The fusion protein of claim 4, wherein the cell surface protein is mutant Nerve Growth Factor Receptor (dNGFR).

6. The fusion protein of claim 1, wherein the scaffold protein is an intracellular protein.

7. The fusion protein of claim 6, wherein the scaffold protein is Green Fluorescent Protein (GFP) or mCherry.

8. A nucleic acid molecule comprising:
a first nucleic acid sequence encoding a fusion protein comprising:
a scaffold protein and
a series of two or more distinct epitopes, wherein the distinct epitopes are recognized by distinct antibodies, and wherein the series of epitopes forms a detectable protein tag and
a first promoter operably linked to the first nucleic acid sequence.

9. The nucleic acid molecule of claim 8, wherein the two or more epitopes are selected from the group consisting of: HA, FLAG, VSVg, V5, AU1, AU5, Strep I, E, E2, Strep II, HSV, protein C tag, S-tag, OLLAS, HAT, and Tag-100-tag.

10. The nucleic acid molecule of claim 8 further comprising:
nucleic acid spacer sequences separating each of the two or more epitopes from each other.

11. The nucleic acid molecule of claim 8, wherein the scaffold protein is a cell surface protein.

12. The nucleic acid molecule of claim 11, wherein the cell surface protein is mutant Nerve Growth Factor Receptor (dNGFR).

13. The nucleic acid molecule of claim 8, wherein the scaffold protein is an intracellular protein.

14. The nucleic acid molecule of claim 13, wherein the scaffold protein is Green Fluorescent Protein (GFP) or mCherry.

15.-19. (canceled)

20. The nucleic acid molecule of claim 8 further comprising:
a second nucleic acid sequence encoding an effector molecule and
a second promoter operatively linked to the second nucleic acid sequence.

21. The nucleic acid molecule of claim 20, wherein the effector molecule is a non-coding regulatory nucleic acid sequence or a protein-coding nucleic acid sequence.

22.-27. (canceled)

28. A vector comprising the nucleic acid molecule of claim 8.

29. (canceled)

30. A method of tracking a cell, said method comprising:
providing a plurality of vectors according to claim 28;
providing a population of cells;
contacting the population of cells with the plurality of vectors under conditions effective for transduction;
contacting the transduced cells with labeling molecules capable of binding the two or more epitopes of each fusion protein of each of the plurality of vectors; and
detecting the labeling molecules to track the transduced cells.

31.-39. (canceled)

40. A kit comprising:
a library of vectors comprising the nucleic acid molecule of claim 8, wherein each vector comprises a different series of two or more distinct epitopes.

41. A kit comprising:
a library of vectors comprising the nucleic acid molecule of claim 20, wherein each vector comprises a different series of two or more distinct epitopes.

42.-43. (canceled)


SEQUENCE LISTING (67 sequences)

SEQ ID NO: 1 - DNA, 77 bp, Artificial Sequence
  Rtp4
caagaactgatgcaggaggagaagcccggggccaagtggagcctgcatttggataagaacattgtaccagatggtgc

SEQ ID NO: 2 - DNA, 40 bp, Artificial Sequence
  Rtp4 KO clone
caagcctgcatttggataagaacattgtaccagatggtgc

SEQ ID NO: 3 - DNA, 68 bp, Artificial Sequence
  Rtp4 KO clone
caagaactgatgcaggaggagaagcccgggagcctgcatttggataagaacattgtaccagatggtgc

SEQ ID NO: 4 - DNA, 67 bp, Artificial Sequence
  Rtp4 KO clone
caagaactgatgcaggaggagaagcccggggcctgcatttggataagaacattgtaccagatggtgc

SEQ ID NO: 5 - DNA, 76 bp, Artificial Sequence
  Rtp4 KO clone
caagaactgatgcaggaggagaagcccggggccaagtgagcctgcatttggataagaacattgtaccagatggtgc

SEQ ID NO: 6 - DNA, 63 bp, Artificial Sequence
  Rtp4 KO clone
caagaactgatgcaggaggagaagcccggggtttttggataagaacattgtaccagatggtgc

SEQ ID NO: 7 - DNA, 66 bp, Artificial Sequence
  Rtp4 KO clone
caagaactgatgcaggaggagaagcccggggccaagtttggataagaacattgtaccagatggtgc

SEQ ID NO: 8 - DNA, 74 bp, Artificial Sequence
  Rtp4 KO clone
caagaactgatgcaggaggagaagcccggggccaagtgcctgcatttggataagaacattgtaccagatggtgc

SEQ ID NO: 9 - DNA, 74 bp, Artificial Sequence
  Rtp4 KO clone
caagaactgatgcaggaggagaagcccggggccaagagcctgcatttggataagaacattgtaccagatggtgc

SEQ ID NO: 10 - DNA, 76 bp, Artificial Sequence; identical to SEQ ID NO: 5
  Rtp4 KO clone

SEQ ID NO: 11 - DNA, 63 bp, Artificial Sequence
  Rtp4 KO clone
caagaactgatgcaggaggagaagcccggggcatttggataagaacattgtaccagatggtgc

SEQ ID NO: 12 - DNA, 67 bp, Artificial Sequence; identical to SEQ ID NO: 4
  Rtp4 KO clone

SEQ ID NO: 13 - DNA, 68 bp, Artificial Sequence; identical to SEQ ID NO: 3
  Rtp4 KO clone

SEQ ID NO: 14 - DNA, 77 bp, Artificial Sequence; identical to SEQ ID NO: 1
  Rtp4 KO clone

SEQ ID NO: 15 - DNA, 74 bp, Artificial Sequence; identical to SEQ ID NO: 8
  Rtp4 KO clone

SEQ ID NO: 16 - DNA, 76 bp, Artificial Sequence; identical to SEQ ID NO: 5
  Rtp4 KO clone

SEQ ID NO: 17 - DNA, 67 bp, Artificial Sequence; identical to SEQ ID NO: 4
  Rtp4 KO clone

SEQ ID NO: 18 - DNA, 76 bp, Artificial Sequence; identical to SEQ ID NO: 5
  Rtp4 KO clone

SEQ ID NO: 19 - DNA, 60 bp, Artificial Sequence
  Rtp4 KO clone
caagaactgatgcaggaggaagagcctgcatttggataagaacattgtaccagatggtgc

SEQ ID NO: 20 - DNA, 77 bp, Artificial Sequence; identical to SEQ ID NO: 1
  Rtp4 KO clone

SEQ ID NO: 21 - protein, 9 aa, Artificial Sequence
  HA tag
YPYDVPDYA

SEQ ID NO: 22 - protein, 8 aa, Artificial Sequence
  FLAG tag
DYKDDDDK

SEQ ID NO: 23 - protein, 11 aa, Artificial Sequence
  VSVg tag
YTDIEMNRLGK

SEQ ID NO: 24 - protein, 14 aa, Artificial Sequence
  V5 tag
GKPIPNPLLGLDST

SEQ ID NO: 25 - protein, 6 aa, Artificial Sequence
  AU1 tag
DTYRYI

SEQ ID NO: 26 - protein, 6 aa, Artificial Sequence
  AU5 tag
TDFYLK

SEQ ID NO: 27 - protein, 9 aa, Artificial Sequence
  S1 tag
NANNPDWDF

SEQ ID NO: 28 - protein, 13 aa, Artificial Sequence
  E tag
GAPVPYPDPLEPR

SEQ ID NO: 29 - protein, 12 aa, Artificial Sequence
  E2 tag
GVSSTSSDFRDR

SEQ ID NO: 30 - protein, 9 aa, Artificial Sequence
  NWS tag
NWSHPQFEK

SEQ ID NO: 31 - protein, 6 aa, Artificial Sequence
  His tag
HHHHHH

SEQ ID NO: 32 - protein, 10 aa, Artificial Sequence
  c-myc tag
EQKLISEEDL

SEQ ID NO: 33 - protein, 12 aa, Artificial Sequence
  protein C tag
EDQVDPRLIDGK

SEQ ID NO: 34 - protein, 15 aa, Artificial Sequence
  Avi tag
GLNDIFEAQKIEWHE

SEQ ID NO: 35 - protein, 6 aa, Artificial Sequence
  B-Tag
QYPALT

SEQ ID NO: 36 - protein, 26 aa, Artificial Sequence
  CBP-tag
KRRWKKNFIAVSAANRFKKISSSGAL

SEQ ID NO: 37 - protein, 8 aa, Artificial Sequence
  DDDDK-tag
  misc_feature (1)..(3): Xaa can be any naturally occurring amino acid
XXXDDDDK

SEQ ID NO: 38 - protein, 6 aa, Artificial Sequence
  Glu-Glu-tag
EYMPME

SEQ ID NO: 39 - protein, 19 aa, Artificial Sequence
  HAT tag
KDHLIHNVHKEFHAHAHNK

SEQ ID NO: 40 - protein, 11 aa, Artificial Sequence
  HSV tag
QPELAPEDPED

SEQ ID NO: 41 - protein, 11 aa, Artificial Sequence
  KT3 tag
KPPTPPPEPET

SEQ ID NO: 42 - protein, 16 aa, Artificial Sequence
  Nano-tag
MDVEAWLGARVPLVET

SEQ ID NO: 43 - protein, 15 aa, Artificial Sequence
  OLLAS
SGFANELGPRLMGKC

SEQ ID NO: 44 - protein, 20 aa, Artificial Sequence
  Rho-tag
MNGTEGPNFYVPFSNKTGVV

SEQ ID NO: 45 - protein, 10 aa, Artificial Sequence
  SRT
TFIGAIATDT

SEQ ID NO: 46 - protein, 15 aa, Artificial Sequence
  S-tag
KETAAAKFERQHMDS

SEQ ID NO: 47 - protein, 11 aa, Artificial Sequence
  T7-tag
MASMTGGQQMG

SEQ ID NO: 48 - protein, 12 aa, Artificial Sequence
  Tag-100-tag
EETARFQPGYRS

SEQ ID NO: 49 - protein, 21 aa, Artificial Sequence
  TAP-tag
CSSGALDYDIPTTASENLYFQ

SEQ ID NO: 50 - protein, 10 aa, Artificial Sequence
  Ty1-tag
EVHTNQDPLD

SEQ ID NO: 51 - protein, 6 aa, Artificial Sequence
  Universal Tag
HTTPHH

SEQ ID NO: 52 - protein, 6 aa, Artificial Sequence
  Polyglycine Linker
GGGGGG

SEQ ID NO: 53 - protein, 8 aa, Artificial Sequence
  Polyglycine Linker
GGGGGGGG

SEQ ID NO: 54 - protein, 6 aa, Artificial Sequence
  Glycine-Serine Linker
GSGSGS

SEQ ID NO: 55 - protein, 5 aa, Artificial Sequence
  Glycine-Serine Linker
GGGGS

SEQ ID NO: 56 - protein, 28 aa, Artificial Sequence
  NGFR signal peptide
MGAGATGRAMDGPRLLLLLLLGVSLGGA

SEQ ID NO: 57 - protein, 19 aa, Artificial Sequence
  Preproalbumin signal peptide
MKWVTFLLLLFISGSAFSR

SEQ ID NO: 58 - protein, 23 aa, Artificial Sequence
  Pre-IgG light chain signal peptide
MDMRAPAQIFGFLLLLFPGTRCD

SEQ ID NO: 59 - protein, 19 aa, Artificial Sequence
  Prelysozyme signal peptide
MRSLLILVLCFLPLAALGK

SEQ ID NO: 60 - protein, 23 aa, Artificial Sequence
  SPtPA signal peptide
MDAMKRGLCCVLLLCGAVFVSPS

SEQ ID NO: 61 - DNA, 20 bp, Artificial Sequence
  Actb forward primer
ctaaggccaaccgtgaaaag

SEQ ID NO: 62 - DNA, 20 bp, Artificial Sequence
  Actb reverse primer
accagaggcatacagggaca

SEQ ID NO: 63 - DNA, 15 bp, Artificial Sequence
  Rtp4 forward primer
cggggccaagtggag

SEQ ID NO: 64 - DNA, 20 bp, Artificial Sequence
  Rtp4 f reverse primer
tggcacaagatcatcacctg

SEQ ID NO: 65 - DNA, 20 bp, Artificial Sequence
  Rtp4 gRNA target
atccaaatgcaggctccact

SEQ ID NO: 66 - DNA, 22 bp, Artificial Sequence
  Forward sequencing primer
tctctcccagatttgaggaaga

SEQ ID NO: 67 - DNA, 20 bp, Artificial Sequence
  Reverse sequencing primer
agcatggggacatggagtac